(19) United States
(12) Patent Application Publication
(10) Pub. No.: US 2007/0149464 A1
(43) Pub. Date: Jun. 28, 2007

(54) COMBINATION

(75) Inventors: Denis Billen, Sandwich (GB); Jessica Boyle, Sandwich (GB); Douglas James Critcher, Sandwich (GB); David Morris Gethin, Sandwich (GB); Kim Thomas Hall, Sandwich (GB); Graham Michael Kyne, Sandwich (GB)

Correspondence Address: WARNER-LAMBERT COMPANY, 2800 PLYMOUTH RD, ANN ARBOR, MI, 48105, US

(73) Assignee: PFIZER INC., New York, NY (US)

(21) Appl. No.: 11/610,852

(22) Filed: Dec. 14, 2006

Related U.S. Application Data
(63) Continuation-in-part of application No. 11/453,053, filed on Jun. 14, 2006.
(60) Provisional application No. 60/690,651, filed on Jun. 15, 2005.

(51) Int. Cl. A61K 31/7048; A61K 31/4152; A61K 31/4439

(52) U.S. Cl. 514/28; 514/341; 514/406; 514/29

(57) ABSTRACT

Compounds of formula (I) are used in combination with a second antiparasitic agent for the treatment of parasitic infestations in a host animal.

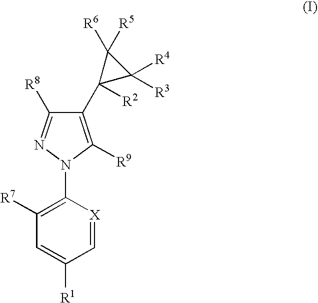

This application is a continuation-in-part of U.S. patent application Ser. No. 11/453,053, filed on 14 Jun. 2006, currently pending, which claims the benefit of U.S. provisional application Ser. No. 60/690,651, filed on 15 Jun. 2005.

The present invention relates to a combination of two antiparasitic agents. In particular it relates to a combination of a 1-aryl-4-cyclopropylpyrazole derivative and an anthelmintic agent. The combination of agents is useful in the treatment of parasitic infestations in animals.

### BACKGROUND

International Patent Application Publication No. (WO) 98/24767, European Patent Application Publication No. (EP) 933363, European Patent Application Publication No. (EP) 959071 and International Patent Application Publication No. (WO) 2005/060749 all describe arylpyrazoles having parasiticidal activity for the control of arthropods.

However, the prior art compounds do not always demonstrate good activity or a long duration of action against parasites. Similarly, some of the prior art parasiticidal agents are useful only for a narrow spectrum of parasites. In some cases this may be attributed to the low bioavailability of the compounds in the treated animal and this can also lead to poor activity. It is an aim of the present invention to overcome various disadvantages of, or improve on, the properties of prior art compounds. Thus it is an aim of the invention to provide an arylpyrazole which has the same or improved activity relative to prior art compounds against parasites. It is a further aim of the present invention to provide arylpyrazole compounds with improved bioavailability whilst maintaining or improving their activity. The compounds of the present invention have especially good ability to control a broad spectrum of arthropods as shown by the results of tests demonstrating their potency and efficacy. In particular, the compounds of the present invention are significantly more active against fleas than similar prior art compounds.

It is a further aim to provide compounds with a long duration of action. Surprisingly it has been found that improving the bioavailability of the compounds does not negatively impact their duration of action. The extended duration of action is generally attributed to an extended half life of the compound in vivo in the host mammal.

It is also desirable that the compounds of the present invention should have an improved pharmacokinetic profile, improved safety, improved persistence and improved solubility.

### SUMMARY OF THE INVENTION

In a first aspect, the present invention provides for a method of treating a parasitic infestation in a host animal, comprising simultaneously, sequentially or separately administering to said host animal:
a) a therapeutically effective amount of a compound according to formula (I) 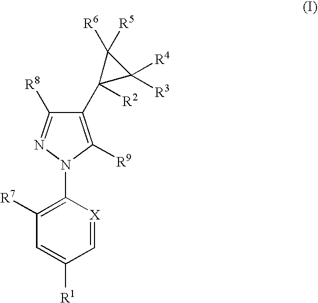
wherein:
X is selected from CR¹⁰ or N;
R¹ is selected from halo, cyano, hydroxy, C₁₋₆ alkyl, C₁₋₆ alkoxy, C₁₋₆ alkanoyl, C₁₋₆ haloalkyl, C₁₋₆ haloalkoxy, C₁₋₆ haloalkanoyl, amino, C₁₋₆ alkyl amino, di C₁₋₆ alkyl amino, het, phenyl, SF₅ and S(O)ₙR¹¹;
R² is selected from cyano, hydroxy, C(O)OH, het, phenyl, S(O)ₙR¹¹, C(O)NR^{a}R^{b} and C(S)NR^{a}R^{b};
or R² is selected from C₃₋₈ cycloalkyl, C₃₋₈ cycloalkyl C₁₋₆ alkyl, C₂₋₆ alkenyl, C₂₋₆ alkynyl, C₁₋₆ alkanoyl, C(O)OC₁₋₆ alkyl, amino, C₁₋₆ alkyl amino, and di C₁₋₆ alkyl amino each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{a} and R^{b} are independently selected from hydrogen, het, phenyl, and S(O)ₙR¹¹;
or either one or both of R^{a} and R^{b} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, and C(O)OC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{a} and R^{b} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R² and R^{e} together with the N atom to which R^{e} is attached may form a six to seven-membered saturated, partially saturated, or unsaturated heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R³, R⁴, R⁵ and R⁶ are independently selected from hydrogen, halo, cyano, hydroxy, C(O)OH, nitro, phenyl, and S(O)ₙR¹¹;
or either one or more of R³, R⁴, R⁵ and R⁶ are independently selected from C₁₋₄ alkyl, C(O)NR^{c}R^{d}, C(S)NR^{c}R^{d}, C₁₋₄ alkoxy, C₁₋₄ alkanoyl, C(O)OC₁₋₄ alkyl, amino which R³, R⁴, R⁵ and R⁶ may be optionally and independently further substituted by one or more substituents selected from, where chemically possible, cyano, nitro, halo, hydroxy, C₁₋₄ alkyl and amino;
and where not more than two of R³, R⁴, R⁵ and R⁶ are selected from cyano, hydroxy, C(O)OH, nitro, phenyl, S(O)ₙR¹¹, C(O)NR^{c}R^{d}, C(S)NR^{c}R^{d}, C₁₋₄ alkoxy, C₁₋₄ alkanoyl, C(O)OC₁₋₄ alkyl, and amino;
R⁷ is selected from halo, C₁₋₆ alkyl and C₁₋₆ alkoxy where, when R⁷ is C₁₋₆ alkyl or C₁₋₆ alkoxy, R⁷ may be optionally substituted with one or more halo substituents;
R⁸ is selected from hydrogen, cyano, hydroxy, C(O)OH, nitro, halo, het, phenyl and S(O)ₙR¹¹;
or R⁸ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₁₋₆ alkanoyl, and C(O)OC₁₋₆ alkyl, which R⁸ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁸ is amino, which R⁸ may be optionally and independently further substituted by one or more substituents selected from, where chemically possible, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, het, phenyl and S(O)ₙR¹¹;
R⁹ is selected from hydrogen, halo, cyano, hydroxy, C(O)OH, nitro, het, phenyl, S(O)ₙR¹¹ and NR^{e}R^{f};
or R⁹ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₃₋₈ cycloalkylC₁₋₆ alkoxy, C₁₋₆ alkanoyl, C(O)OC₁₋₆ alkyl, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{e} and R^{f} are independently selected from hydrogen, het, phenyl and S(O)ₙR¹¹;
or either one or both of R^{e} and R^{f} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}CR^{d}, het, phenyl and S(O)ₙR¹¹;
or R^{e} and R^{f} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}CR^{d}, het, phenyl and S(O)ₙR¹¹;
or R^{e} and R² together with the atoms to which they are attached may form a six to seven-membered heterocyclic ring as previously described;
R¹⁰ is selected from halo, C₁₋₆ alkyl and C₁₋₆ alkoxy and where when R¹⁰ is C₁₋₆ alkyl or C₁₋₆ alkoxy it may optionally be substituted with one or more halo substituents;
each of R^{c}and R^{d} are independently selected from hydrogen, C₁₋₆ alkyl, C₂₋₆ alkenyl, C₃₋₈ cycloalkyl, C₃₋₈ cycloalkylC₁₋₆ alkyl, C₁₋₆ haloalkyl, C₃₋₈ cycloalkylC₁₋₆ haloalkyl, C₁₋₆ alkanoyl, C₁₋₆ haloalkanoyl, C(O)OC₁₋₆ alkyl, het, phenyl and S(O)ₙR¹¹;
or R^{c} and R^{d} together with the N atom to which at least one of them is attached may form a three to seven-membered saturated, partially saturated, unsaturated or aromatic heterocyclic ring which may optionally contain one or more further N, O or S atoms;
each n is independently 0, 1 or 2;
each R¹¹ is independently selected from hydrogen, hydroxy, C₁₋₆ alkyl, C₁₋₆ haloalkyl, amino, C₁₋₆ alkyl amino and di C₁₋₆ alkyl amino;
each phenyl may be optionally substituted by one or more further substitutents selected from the group consisting of halo, cyano, nitro, hydroxy, C₁₋₆ alkyl, C₁₋₆ haloalkyl, C₁₋₆ alkoxy, C₁₋₆ haloalkoxy, amino, C₁₋₆ alkyl amino, di C₁₋₆ alkyl amino, —NHS(O)ₙR¹¹, and S(O)ₙR¹¹;
and each het independently represents a four to seven membered heterocyclic ring, which is aromatic or non-aromatic, unsaturated, partially saturated or saturated and which contains one or more heteroatoms selected from nitrogen, N-oxide, oxygen, sulphur and wherein said heterocyclic ring is optionally substituted, where the valence allows, with one or more substituents selected from halo, cyano, nitro, C₁₋₆ alkyl, C₁₋₆ haloalkyl, C₁₋₆ alkoxy, OC(O) C₁₋₆ alkyl, C₁₋₆ alkanoyl, C(O)O C₁₋₆ alkyl and NR^{g}R^{h}, where R^{g} and R^{h} are independently selected from hydrogen, C₁₋₆ alkyl and C₂₋₆ alkenyl, and where each of the above groups may include one or more optional substituents where chemically possible independently selected from cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, C₁₋₆ alkyl amino, di C₁₋₆ alkyl amino, phenyl and S(O)ₙR¹¹;
or a pharmaceutically acceptable salt or a prodrug thereof; and
   b) a therapeutically effective amount of a second antiparasitic agent.

In a second aspect, the invention provides a pharmaceutical composition for the treatment of a parasitic infestation, comprising a compound of formula (I) as defined above, or a pharmaceutically acceptable salt or a prodrug thereof; and a second antiparasitic agent.

In a further aspect, the invention provides a kit for treating a parasitic infestation in a host animal, comprising a pharmaceutical composition comprising a therapeutically effective amount of a compound according to formula (I) as defined above, or a pharmaceutically acceptable salt or a prodrug thereof; and a pharmaceutical composition comprising a therapeutically effective amount of a second antiparasitic agent.

### DETAILED DESCRIPTION OF THE INVENTION

In a first aspect, the invention relates to a method of treating a parasitic infestation in a host animal.

For the avoidance of doubt, references herein to “treatment” or “treating” as used herein includes references to curative, palliative and prophylactic treatment, and to controlling the parasites including killing, repelling, expelling, incapacitating, deterring, eliminating, alleviating, minimising, and eradicating the parasite.

Infestations susceptible to control and/or treatment according to the method of the invention include infestations by parasites such as arthropods and helminths. Examples of arthropods include Acarina, including ticks (e.g. Ixodes spp., Boophilus spp. e.g. Boophilus microplus, Amblyomma spp., Hyalomma spp., Rhipicephalus spp. e.g. Rhipicephalus appendiculatus, Haemaphysalis spp., Dermacentor spp., Ornithodorus spp. (e.g. Omithodorus moubata), mites (e.g. Damalinia spp., Dermanyssus gallinae, Sarcoptes spp. e.g. Sarcoptes scabiei, Psoroptes spp., Chorioptes spp., Demodex spp., Eutrombicula spp.); Diptera (e.g. Aedes spp., Anopheles spp., Muscidae spp. e.g. Stomoxys calcitrans and Haematobia irritans, Hypoderma spp., Gastrophilus spp., Simulium spp.); Hemiptera (e.g. Triatoma spp.); Phthiraptera (e.g. Damalinia spp., Linognathus spp.); Siphonaptera (e.g. Ctenocephalides spp.); Dictyoptera (e.g. Periplaneta spp., Blatella spp.) and Hymenoptera (e.g. Monomorium pharaonis). Examples of helminths include parasites of the phylum Platyhelminthes (such as cestodes and trematodes; e.g. Fasciola spp.; Fascioloides spp.; Paramphistomum spp.; Dicrocoelium spp.; Eurytrema spp.; Ophisthorchis spp.; Fasciolopsis spp.; Echinostoma spp.; Paragonimus spp.) and the phylum Nematoda (such as filarial, intestinal and tissue nematodes; e.g. Haemonchus spp.; Ostertagia spp.; Cooperia spp.; Oesphagastomum spp.; Nematodirus spp.; Dictyocaulus spp.; Trichuris spp.; Toxocara spp.; Toxascaris spp.; Trichinella spp.; Dirofilaria spp.; Ancyclostoma spp.; Necator spp.; Strongyloides spp.; Capillaria spp.; Ascaris spp.; Enterobius spp.; and Trichostrongylus spp.).

The method of the invention is particularly suited to the treatment of host animals that are subject to, or at risk of, parasitic infestations by two parasites simultaneously.

The host animal may be a mammal or a non-mammal, such as a bird or a fish. Where the host animal is a mammal, it may be a human or non-human mammal. Non-human mammals include livestock animals and companion animals, such as cattle, sheep, goats, equines, swine, dogs and cats.

The method of the invention is of particular value in the control of arthropods which are injurious to, or spread or act as vectors of diseases in, man and domestic animals, for example those hereinbefore mentioned, and more especially in the control of ticks, mites, lice, fleas, midges and biting, nuisance and myiasis flies. It is particularly useful in controlling arthropods which are present inside domestic host animals or which feed in or on the skin or suck the blood of the animal.

The method of the invention is of value for the treatment and control of the various lifecycle stages of parasites including egg, nymph, larvae, juvenile and adult stages.

The method comprises the administration of two pharmacologically active components to the host animal.

1.1-Aryl-4-cyclopropylpyrazole Component

1-Aryl-4-cyclopropylpyrazole derivatives according to general formula (I) are described in International Patent Application PCT/IB2006/001582, which is incorporated herein by reference in its entirety.

Preferably, R¹ is selected from: cyano; C₁₋₆ haloalkyl, for example, trifluoromethyl or i-C₃F₇; C₁₋₆ haloalkoxy, for example, difluoromethoxy or trifluoromethoxy; SF₅; and S(O)ₙR¹¹ where, for example, R¹¹ is C₁₋₆ haloalkyl to form, for example, (trifluoromethyl)thio, (trifluoromethyl)sulphinyl or (trifluoromethyl)sulphonyl. More preferably R¹ is selected from C₁₋₆ haloalkyl, for example, trifluoromethyl, C₁₋₆ haloalkoxy for example difluoromethoxy and trifluoromethoxy, and SF₅. Even more preferably R¹ is selected from CF₃, OCF₃, or SF₅. Most preferably R¹ is SF₅.

Suitably, R² is selected from: cyano; C(O)OH; het, eg 1-oxa-3,4-diazolyl or thiazolyl, which het may in turn be substituted with C₁₋₆ alkyl, eg methyl or ethyl to form, for example, 5-methyl-1-3,4-oxadiazol-2-yl; and S(O)ₙR¹¹ where R¹¹ is selected from C₁₋₆ alkyl, eg methyl or ethyl to form, for example, methylthio, methylsulphinyl or methylsulphonyl, amino to form, for example, aminosulphonyl, and di C₁₋₆ alkyl amino, eg dimethylamino to form, for example, (dimethylamino)sulphonyl; C(O)OC₁₋₆ alkyl, eg methoxycarbonyl or ethoxycarbonyl, which C(O)OC₁₋₆ alkyl may in turn be optionally substituted with halo, eg chloro or fluoro to form, for example, fluoromethoxycarbonyl or trifluoromethoxycarbonyl; and amino.

Equally suitably R² is selected from C(O)NR^{a}R^{b} and C(S)NR^{a}R^{b} where R^{a} and R^{b} are independently selected from: hydrogen to form, for example, aminocarbonyl or aminocarbonothioyl; S(O)ₙR¹¹ where R¹¹ is C₁₋₆ alkyl, eg methyl or ethyl to form, for example, [(methylsulphonyl)amino]carbonyl; and C₃₋₈ cycloalkyl, eg cyclopropyl to form, for example, (cyclopropylamino)carbonyl. Equally suitably R^{a} and R^{b} are independently selected from C₁₋₆ alkyl, eg methyl, ethyl, propyl, isopropyl or isobutyl to form, for example, (methylamino)carbonyl, (dimethylamino)carbonyl, (ethylamino)carbonyl, (propylamino)carbonyl, (isopropylamino)carbonyl, or (isobutylamino)carbonyl, which C₁₋₆ alkyl may in turn be optionally substituted with one or more substituents selected from: halo eg fluoro to form, for example, [(trifluromethyl)amino]carbonyl or [(2,2,2-trifluoroethyl)amino]carbonyl; hydroxy to form, for example, [(2-hydroxyethyl)amino]carbonyl or [(2-hydroxy-2-methylpropyl)amino]carbonyl; C₁₋₆ alkoxy to form, for example, [(1-methoxyethyl)amino]carbonyl or [(1-isopropoxypropyl)amino]carbonyl; C₃₋₈ cycloalkyl, eg cyclopropyl to form, for example, [(cyclopropylmethyl)amino]carbonyl; or het, eg pyridinyl to form, for example, [(pyridin-2-ylmethyl)amino]carbonyl, [(pyridin-3-ylmethyl)amino]carbonyl, or [(pyridin-4-ylmethyl)amino]carbonyl, or 1,2,4 triazolyl to form, for example, [(4H-1,2,4-triazol-3ylmethyl)amino]carbonyl, which 1,2,4 triazolyl may optionally be further substituted with, for example, C₁₋₆ alkyl, eg methyl to form, for example, {[(5-methyl-4H-1,2,4-triazol-3-yl)methyl]amino}carbonyl.

Where R^{a} and R^{b} together with the N atom to which they are attached form a three to seven-membered saturated, partially saturated, unsaturated or aromatic heterocyclic ring which may optionally contain one or more further N, O or S atoms, the ring is suitably a saturated pyrrolidinyl ring.

Where R² and R^{e} together with the N atom to which R^{e} is attached form a six to seven-membered saturated, partially saturated, or unsaturated heterocyclic ring which may optionally contain one or more further N, O or S atoms it is preferred that R² is selected from C(O)NR^{a}R^{b} and C(S)NR^{a}R^{b} wherein it is then R^{a} and R^{e} together with the N atoms to which they are attached form a six to seven-membered saturated, partially saturated, or unsaturated heterocyclic ring which may optionally contain one or more further N, O or S atoms. Suitably the ring is a partially unsaturated 1,3-diazepanyl which may be further substituted by C₁₋₆ alkyl, eg methyl to form, for example, a 7′-methyl-5′-oxo-5′,6′,7′,8′-tetrahydro-pyrazolo[3,4-d][1,3]diazepine.

Preferably R² is selected from: cyano; C(O)OH; het, eg 1-oxa-3,4-diazolyl or thiazolyl, which 1-oxa-3,4-diazolyl may in turn be substituted with C₁₋₆ alkyl, eg methyl; S(O)ₙR¹¹ where R¹¹ is selected from C₁₋₆ alkyl, eg methyl or ethyl, amino, and di C₁₋₆ alkyl amino; C(O)OC₁₋₆ alkyl, eg methoxycarbonyl or ethoxycarbonyl, which C(O)OC₁₋₆ alkyl may in turn be optionally substituted with halo, eg chloro or fluoro; and amino. Further preferred compounds include those where R² is selected from C(O)NR^{a}R^{b} and C(S)NR^{a}R^{b} where R^{a} and R^{b} are independently selected from: hydrogen; S(O)ₙR¹¹ where R¹¹ is C₁₋₆ alkyl, eg methyl or ethyl; C₃₋₈ cycloalkyl eg cyclopropyl; and C₁₋₆ alkyl, eg methyl, ethyl, isopropyl or isobutyl which C₁₋₆ alkyl may in turn be optionally substituted with one or more groups selected from halo eg fluoro, hydroxy, C₁₋₆ alkoxy, C₃₋₈ cycloalkyl, eg cyclopropyl, or het, eg pyridinyl, or 1, 2, 4 triazolyl which 1, 2, 4 triazolyl may optionally be further substituted with, for example, C₁₋₆ alkyl eg methyl.

Even more preferably R² is selected from: cyano; S(O)ₙR¹¹ where R¹¹ is C₁₋₆ alkyl, eg methyl or ethyl; and C(O)NR^{a}R^{b}, where R^{a} is hydrogen and R^{b} is selected from hydrogen, and C₁₋₆ alkyl eg methyl or isopropyl, which C₁₋₆ alkyl may be optionally substituted with het, eg pyridinyl to form, for example, [(pyridin-4-ylmethyl)amino]carbonyl.

Most preferably, R² is C(O)NR^{a}R^{b} where both of R^{a} and R^{b} are hydrogen.

Suitably R³, R⁴, R⁵ and R⁶ are each independently selected from: hydrogen; halo, eg chloro or fluoro; or C₁₋₄ alkyl, eg methyl, which C₁₋₄ alkyl is optionally substituted by 1 to 5 halo groups independently selected from chloro or fluoro to form, for example, trifluoromethyl. Preferably, R³ and R⁴ are independently selected from: hydrogen; chloro; fluoro; and C₁₋₄ alkyl, eg methyl which C₁₋₄ alkyl is optionally substituted by 1 to 5 halo groups and both R⁵ and R⁶ are hydrogen. More preferably, both R³ and R⁴ are the same as each other and are selected from: hydrogen; fluoro; chloro; and methyl and both R⁵ and R⁶ are hydrogen. Most preferably, both R³ and R⁴ are the same as each other and are selected from: hydrogen; fluoro; and chloro and both R⁵ and R⁶ are hydrogen.

Suitable compounds include those where, when R⁷ is halo, preferred halo substituents are fluoro, chloro or bromo. Further suitable compounds include those where, when R⁷ is selected from C₁₋₆ alkyl or C₁₋₆ alkoxy where the C₁₋₆ alkyl or C₁₋₆ alkoxy are optionally substituted with one or more halo substituents, preferred halo substituents are fluoro, chloro or bromo. Preferably R⁷ is selected from chloro, or fluoro. Most preferably R⁷ is chloro.

Suitably, R⁸ is selected from: cyano; halo, eg chloro or fluoro; C₁₋₆ alkyl, eg methyl or ethyl which C₁₋₆ alkyl may optionally be substituted with one or more fluoro groups to form, for example, trifluoromethyl; and C₁₋₆ alkanoyl, eg acetyl or propanoyl which C₁₋₆ alkanoyl may optionally be substituted by one or more substituents independently selected from S(O)ₙR¹¹ eg where R¹¹ is C₁₋₆ alkyl, eg methyl or ethyl to form, for example, (methylthio)carbonyl, halo eg chloro or fluoro, to form for example trifluoroacetyl, or C₁₋₆ alkoxy to form, for example 2-ethoxy-2-oxoethyl.

Preferably, R⁸ is selected from: cyano; C₁₋₆ alkyl, eg methyl which C₁₋₆ alkyl may optionally be substituted with one or more fluoro groups; and C₁₋₆ alkanoyl, eg acetyl which C₁₋₆ alkanoyl may optionally be substituted by S(O)ₙR¹¹, eg where R¹¹ is C₁₋₆ alkyl. Most preferably, R⁸ is cyano.

Suitably R⁹ is selected from: hydrogen; hydroxy; cyano; halo, eg chloro or fluoro; het, eg pyrazinyl, imidazolyl, or pyridinyl to form, for example, pyridin-2-yl or pyridin-4-yl, where suitably the pyridinyl may be further substituted with, eg oxy to form, for example, 1-hydroxy-pyridinyl; phenyl which phenyl may in turn be optionally substituted by one or more substituents selected from: halo, eg chloro or fluoro to form, for example, 4-fluorophenyl or 3,4-difluorophenyl, and S(O)ₙR¹¹, eg where R¹¹ is methyl to form, for example, 4-(methylsulphonyl)phenyl; and S(O)ₙR¹¹, eg where R¹¹ is methyl to form, for example, methylthio, methylsulphinyl, or methylsulphonyl.

Further suitable compounds include those where R⁹ is C₁₋₆ alkyl, eg methyl, ethyl, isopropyl, or t-butyl which C₁₋₆ alkyl may in turn optionally be substituted by one or more substituents selected from: halo, eg fluoro or chloro to form, for example, difluoromethyl, trifluoromethyl or trifluoroethyl; C₁₋₆ alkyl, eg t-butyl to form, for example, t-butylmethyl; C₃₋₈ cycloalkyl, eg cyclopropyl, cyclopentyl or cyclohexyl to form, for example, cyclopropylmethyl, cyclopentylmethyl, cyclohexylmethyl or cyclopropylethyl; C₁₋₆ alkoxy, eg methoxy or ethoxy to form, for example, methoxymethyl, methoxyethyl, ethoxymethyl or ethoxyethyl; het, eg pyrazinyl to form, for example, pyrazinylmethyl or pyrazinylethyl, imidazolyl to form, for example, (1H-imidazolyl)methyl or (1H-imidazolyl)ethyl, 1,2,4-triazolyl to form, for example, (4H-1,2,4-triazol-3-yl methyl or (4H-1,2,4-triazol-3-yl)ethyl, or pyridinyl to form, for example, pyridin-2-ylmethyl, pyridin-2-ylethyl, pyridin-4-ylmethyl or pyridin-4-ylethyl, where suitably the pyridinyl may be further substituted with, eg oxy to form, for example, (1-hydroxy-pyridinyl)methyl or (1-hydroxy-pyridinyl)ethyl; phenyl to form, for example, benzyl or phenylethyl which phenyl may in turn be optionally substituted by one or more substituents selected from halo, eg chloro or fluoro to form, for example, 4-fluorobenzyl, (4-fluorophenyl)ethyl, 3,4-difluorobenzyl or (3,4-difluorophenyl)ethyl, C₁₋₄ alkyl optionally substituted by one or more halo groups, eg chloro or fluoro to form, for example, (trifluoromethyl)benzyl or [(trifluoromethyl)phenyl]ethyl, or S(O)ₙR¹¹, eg where R¹¹ is methyl to form, for example, 4-(methylsulphonyl)benzyl or [4-(methylsulphonyl)phenyl]ethyl; —C(O)O C₁₋₆ alkyl eg ethoxycarbonyl to form, for example, 2-ethoxy-2-oxoethyl; amino to form for example aminomethyl or aminoethyl; C₁₋₆ alkyl amino, eg methylamino to form, for example, (methylamino)methyl, (methylamino)ethyl, (ethylamino)methyl or (ethylamino)ethyl; and S(O)ₙR¹¹, eg where R¹¹ is methyl to form, for example, (methylthio)methyl, (methylthio)ethyl, (methylsulphinyl)methyl, (methylsulphinyl)ethyl, (methylsulphonyl)methyl, or (methylsulphonyl)ethyl.

Further suitable compounds include those where R⁹ is selected from: C₂₋₆ alkenyl, eg ethenyl which C₂₋₆ alkenyl may be further substituted with het eg pyrazinyl, 1,3,4-triazolyl, imidazolyl, or pyridinyl, or phenyl which phenyl may be further substituted by for example halo, eg chloro or fluoro to form, for example, 4-fluorophenyl or 3,4-difluorophenyl, C₁₋₄ alkyl optionally substituted by one or more halo groups, eg chloro or fluoro to form, for example, trifluoromethylphenyl, or S(O)ₙR¹¹, eg where R¹¹ is methyl to form, for example, 4-(methylsulphonyl)phenyl; C₃₋₈ cycloalkyl, eg cyclopropyl, cyclobutyl, cyclopentyl or cyclohexyl, which C₃₋₈ cycloalkyl may be optionally substituted with one or more groups selected from halo, eg fluoro or chloro, cyano, and hydroxy; and C₃₋₈ cycloalkylC₁₋₆ alkyl, eg cyclopropylmethyl or cyclopropylethyl, which C₃₋₈ cycloalkylC₁₋₆ alkyl may be optionally substituted with one or more groups selected from halo eg fluoro or chloro, to form, for example, (1-fluorocyclopropyl)methyl, C₁₋₆ alkyl eg methyl or ethyl to form, for example, (1-methylcyclopropyl)methyl or (1-ethylcyclopropyl)methyl, and C₁₋₆ haloalkyl to form, for example, [(1-trifluoromethyl)cyclopropyl]methyl.

Equally suitably R⁹ is C₁₋₆ alkoxy, eg methoxy, ethoxy, isopropoxy or t-butoxy which C₁₋₆ alkoxy may in turn optionally be substituted by one or more substituents selected from: halo, eg fluoro or chloro to form, for example, trifluoromethoxy or trifluoroethoxy; C₁₋₆ alkyl, eg t-butyl to form, for example, t-butylmethoxy; C₃₋₈ cycloalkyl, eg cyclopropyl, cyclopentyl or cyclohexyl to form, for example, cyclopropylmethoxy, cyclopentylmethoxy, cyclohexylmethoxy or cyclopropylethoxy; het, eg pyrazinyl to form, for example, pyrazinylmethoxy, imidazolyl to form, for example, (1H-imidazolyl)methoxy, 1,3,4-triazolyl to form, for example, (4H-1,2,4-triazol-3-yl)methoxy or (4H-1,2,4-triazol-3-yl)ethoxy, or pyridinyl to form, for example, pyridin-2-ylmethoxy or pyridin-4-ylmethoxy, where suitably the pyridinyl may be further substituted with, eg oxy to form, for example, (1-hydroxypyridinyl)methoxy; phenyl to form, for example, benzyloxy which phenyl may in turn be optionally substituted by one or more substituents selected from halo, eg chloro or fluoro to form, for example, (4-fluorobenzyl)oxy or (3,4-difluorobenzyl)oxy, C₁₋₄ alkyl optionally substituted by one or more halo groups, eg chloro or fluoro to form, for example, [(trifluoromethyl)benzyl]oxy, and S(O)ₙR¹¹, eg where R¹¹ is methyl to form, for example, [4-(methylsulphonyl)benzyl]oxy; and —C(O)O C₁₋₆ alkyl, eg ethoxycarbonyl to form, for example, 2-ethoxy-2-oxoethyl.

Equally suitably R⁹ is C₃₋₈ cycloalkylC₁₋₆ alkoxy eg cyclopropylmethoxy or cyclopropylethoxy which C₃₋₈ cycloalkylC₁₋₆ alkoxy may be optionally substituted with one or more groups selected from: halo eg fluoro or chloro, to form for example (1-fluorocyclopropyl)methoxy; C₁₋₆ alkyl eg methyl or ethyl to form, for example (1-methylcyclopropyl)methoxy or (1-ethylcyclopropyl)methoxy; or C₁₋₆ haloalkyl to form, for example, [1-(trifluoromethyl)cyclopropyl]methoxy.

Still further suitable compounds include those where R⁹ is NR^{e}R^{f} and where each of R^{e} and R^{f} are hydrogen to form, for example, amino.

Still further suitable compounds include those where R⁹ is NR^{e}R^{f} and where each of R^{e} or R^{f} are independently selected from hydrogen and C₁₋₆ alkyl, eg methyl, ethyl, n-propyl, isopropyl, n-butyl, t-butyl, or n-pentyl to form, for example, methylamino, dimethylamino, ethylamino, propylamino, isopropylamino, butylamino, t-butylamino, or pentylamino which C₁₋₆ alkyl may in turn be substituted with one or more substituents selected from: cyano to form, for example, (2-cyanoethyl)amino; halo, eg fluoro or chloro to form, for example, (fluoroethyl)amino, (2-fluoro-2-methyl)propylamino, (trifluoromethyl)amino, (trifluoroethyl)amino, (2-fluoroethyl)amino, (3,3,3-trifluoropropyl)amino, (4,4,4-trifluorobutyl)amino, or (5,5,5-trifluoropentyl)amino; C(O)OH to form, for example, (3-carboxypropyl)amino; C(O)NR^{c}R^{d} where R^{c} or R^{d} are independently selected from the group consisting of hydrogen to form, for example, 2-carbamoyl-ethylamino, 3-carbamoyl-propylamino, or 4-carbamoyl-butylaminoamino, C₃₋₈ cycloalkylC₁₋₆ alkyl eg cyclopropylmethyl to form, for example, (2-cyclopropylmethyl-carbamoyl)ethylamino, or C₁₋₆ haloalkyl eg trifluoroethyl to form, for example, (trifluoroethyl-carbamoyl)ethylamino; C₁₋₆ alkyl, eg methyl, isopropyl, t-butyl to form, for example, isopropylmethylamino, or t-butylmethylamino; C₁₋₆ alkoxy, eg methoxy, ethoxy or isopropoxy to form, for example, (2-methoxyethyl)(methyl)amino or (2-isopropoxyethyl)amino; het, eg pyrazinyl to form, for example, pyrazinylmethylamino, imidazolyl to form, for example, (1H-imidazol-2-yl)methylamino, 1,2,4-triazolyl to form, for example, (4H-1,2,4-triazol-3-yl)methylamino, (4H-1,2,4-triazol-3-yl)ethylamino, or (4H-1,2,4-triazol-1-yl)ethylamino, isoxaolyl to form, for example, isoxazol-3-ylmethylamino, thiazolyl to form, for example, 1,3-thiazol-2-ylmethylamino or 1,3-thiazol-4-ylmethylamino which thiazolyl may be optionally further substituted with halo, eg chloro to form, for example, [(2-chloro-1,3-thiazol-4-yl)methyl]amino, pyrazolyl to form, for example, (1H-pyrazol-4-ylmethyl)amino or (1H-pyrazol-4-ylethyl)amino which pyrazolyl may be optionally further substituted with one or more substituents selected from C₁₋₆alkyl, eg methyl, or halo, eg chloro, to form, for example, [(1-methyl-1H-pyrazol-4-yl)ethyl]amino, or [(1-methyl-3-methyl-5-chloro-1H-pyrazol-4-yl)methyl]amino, tretrahydropyranyl to form, for example, (tetrahydro-2H-pyran-4-ylmethyl)amino, or pyridinyl to form, for example, (pyridin-2-ylmethyl)amino or (pyridin-4-ylmethyl)amino, where suitably the pyridinyl may be further substituted with, eg oxy to form, for example, [(1-hydroxypyridin-4-yl)methyl]amino; phenyl to form, for example, benzylamino which phenyl may in turn be optionally substituted by one or more substituents selected from halo, eg chloro or fluoro to form, for example, (4-fluorobenzyl)amino or (3,4-difluorobenzyl)amino, C₁₋₆ alkyl optionally substituted by one or more halo groups, eg chloro or fluoro to form, for example, (trifluoromethylbenzyl)amino, S(O)ₙR¹¹, eg where R¹¹ is methyl to form, for example, [(4-methylsulphonyl)benzyl]amino, or where R¹¹ is C₁₋₆ alkyl amino eg N-methyl to form, for example, {4-[(methylsulphonyl)amino]benzyl}aminoamino, —NHS(O)ₙR¹¹, eg where R¹¹ is methyl to form, for example, {4-[(methylamino)sulphonyl]benzyl}aminoamino; and S(O)ₙR¹¹ eg where R¹¹ is methyl to form, for example, 3-(S-methyl thio ether) propyl amino.

Yet further suitable compounds include those R^{e} is independently selected from hydrogen or C₁₋₆ alkyl, eg methyl and R^{f} is independently selected from: C₃₋₈ cycloalkyl, eg cyclopropyl to form, for example, cyclopropylamino; and C₃₋₈ cycloalkylC₁₋₆ alkyl eg cyclopropylmethyl, cyclopropylethyl, cyclobutylmethyl, cyclopentylmethyl or cyclohexylmethyl to form, for example, (cyclopropylmethyl)amino, (cyclopropylmethyl)(methyl)amino, (cyclopropylethyl)amino, (cyclobutylmethyl)amino, (cyclopentylmethyl)amino or (cyclohexylmethyl)amino, which C₃₋₈ cycloalkylC₁₋₆ alkyl may be optionally substituted with one or more groups selected from: halo eg fluoro or chloro, to form for example [(1-fluorocyclopropyl)methyl]amino; C₁₋₆ alkyl eg methyl or ethyl to form, for example, [(1-methylcyclopropyl)methyl]amino or [(1-ethylcyclopropyl)methyl]amino; C₁₋₆ haloalkyl eg trifluoromethyl to form, for example, [(1-trifluoromethylcyclopropyl)methyl]amino; amino to form, for example, [(1-aminocyclopropyl)methyl]amino; C(O)NR^{c}R^{d} where R^{c} and R^{d} are hydrogen to form, for example, {[1-(aminocarbonyl)cyclopropyl]methyl}amino; NR^{c}R^{d} where R^{c} or R^{d} are independently selected from the group consisting of hydrogen, C(O)OC₁₋₆ alkyl eg t-butoxycarbonyl, or S(O)ₙR¹¹ where R¹¹ is methyl to form, for example, {{1-[(t -butoxycarbonyl)amino]cyclopropyl}methyl}amino, or {{1-[(methylsulphonyl)amino]cyclopropyl}methyl}amino.

Yet further suitable compounds include those where R^{e} is independently selected from hydrogen or C₁₋₆ alkyl, eg methyl and R^{f} is independently selected from: —C(O)O C₁₋₆ alkyl, eg methoxycarbonyl, ethoxycarbonyl or isopropoxycarbonyl to form, for example, (methoxycarbonyl)amino, (ethoxycarbonyl)amino, (isopropoxycarbonyl)amino or (methyl)(isopropoxycarbonyl)amino; —C(O)OC₃₋₈ cycloalkyl eg cyclobutoxycarbonyl to form, for example, (cyclobutyloxycarbonyl)amino or (methyl)(cyclobutyloxycarbonyl)amino; and —C(O)O C₁₋₆ alkylC₃₋₈ cycloalkyl eg cyclopropylmethoxycarbonyl to from, for example, [(cyclopropylmethoxy)carbonyl]amino or (methyl)[(cyclopropylmethoxy)carbonyl]amino, which —C(O)O C₁₋₆ alkylC₃₋₈ cycloalkyl may be further optionally substituted by, for example, C₁₋₆ haloalkyl eg fluoromethyl to form, for example, {{[1-(fluoromethyl)cyclopropyl]methoxy}carbonyl}amino.

Preferably R⁹ is selected from: hydrogen; halo, eg chloro; C₁₋₆ alkyl, eg methyl, which C₁₋₆ alkyl may in turn optionally be substituted by one or more substituents selected from halo, eg fluoro to form, for example, difluoromethyl, or C₁₋₆ alkoxy, eg methoxy to form, for example, methoxymethyl; C₂₋₆ alkenyl, eg ethyenyl; C₃₋₈ cycloalkylC₁₋₆ alkoxy eg cyclopropylmethoxy; and S(O)ₙR¹¹, eg where R¹¹ is methyl to form, for example, methylthio, methylsulphinyl, or methylsulphonyl.

Equally preferred compounds include those where R⁹ is NR^{e}R^{f} where each of R^{e} or R^{f} are independently selected from hydrogen and C₁₋₆ alkyl, eg methyl, ethyl, n-propyl, isopropyl, n-butyl, t-butyl, or n-pentyl which C₁₋₆ alkyl may in turn be substituted with one or more substituents selected from: cyano; halo, eg fluoro; C(O)OH; C(O)NR^{c}R^{d} where R^{c} or R^{d} are independently selected from the group consisting of hydrogen, C₃₋₈ cycloalkylC₁₋₆ alkyl eg cyclopropylmethyl, or C₁₋₆ haloalkyl eg trifluoroethyl; C₁₋₆ alkyl, eg methyl, isopropyl, t-butyl; C₁₋₆ alkoxy, eg methoxy, ethoxy or isopropoxy; het, eg pyrazinyl, imidazolyl, 1,2,4-triazolyl, isoxaolyl, thiazolyl which thiazolyl may be optionally further substituted with halo, eg chloro, pyrazolyl which pyrazolyl may be optionally further substituted with C₁₋₆alkyl, eg methyl or halo, eg chloro, tretrahydropyranyl, or pyridinyl where suitably the pyridinyl may be further substituted with eg oxy; phenyl which phenyl may in turn be optionally substituted by one or more substituents selected from halo, eg fluoro, C₁₋₆ alkyl optionally substituted by one or more halo groups, eg fluoro, S(O)ₙR¹¹, eg where R¹¹ is methyl or where R¹¹ is C₁₋₆ alkyl amino eg N-methyl, —NHS(O)ₙR¹¹, eg where R¹¹ is methyl; and S(O)ₙR¹¹ eg where R¹¹ is methyl.

Equally preferred compounds include those where R⁹ is NR^{e}R^{f} where R^{e} is hydrogen or C₁₋₆ alkyl, eg methyl and R^{f} is C₃₋₈ cycloalkylC₁₋₆ alkyl eg cyclopropylmethyl, cyclopropylethyl, cyclobutylmethyl, cyclopentylmethyl or cyclohexylmethyl, which C₃₋₈ cycloalkylC₁₋₆ alkyl may be optionally substituted with one or more groups selected from: C₁₋₆ alkyl eg methyl; amino; C(O)NR^{c}R^{d} where R^{c} and R^{d} are both hydrogen; and NR^{c}R^{d} where R^{c} and R^{d} are independently selected from the group consisting of hydrogen, C(O)OC₁₋₆ alkyl eg t-butoxy carbonyl, and S(O)ₙR¹¹ where R¹¹ is methyl.

Equally preferred compounds include those where R⁹ is NR^{e}R^{f} where R^{e} is hydrogen or C₁₋₆ alkyl, eg methyl and R^{f} is selected from: —C(O)O C₁₋₆ alkyl, eg methoxycarbonyl, ethoxycarbonyl or isopropoxycarbonyl; —C(O)OC₃₋₈ cycloalkyl eg cyclobutoxycarbonyl; and —C(O)OC₁₋₆ alkylC₃₋₈ cycloalkyl eg cyclopropylmethoxycarbonyl, which —C(O)OC₁₋₆ alkylC₃₋₈ cycloalkyl may be further optionally substituted by, for example, C₁₋₆ haloalkyl eg fluoromethyl.

Even more preferably R⁹ is selected from: halo eg chloro; C₁₋₆ alkyl, eg methyl, which C₁₋₆ alkyl may in turn optionally be substituted by halo, eg fluoro; NR^{e}R^{f} where each of R^{e} or R^{f} is independently selected from hydrogen, C₁₋₆ alkyl, eg methyl, ethyl, n-propyl, isopropyl, n-butyl, t-butyl, or n-pentyl which C₁₋₆ alkyl may in turn be substituted with one or more substituents selected from cyano, halo, eg fluoro, C(O)NR^{c}R^{d} where R^{c} and R^{d} are both hydrogen, het, eg 1,2,4-triazolyl, or S(O)ₙR¹¹ eg where R¹¹ is methyl; C₃₋₈ cycloalkylC₁₋₆ alkyl eg cyclopropylmethyl, cyclopropylethyl, which C₃₋₈ cycloalkylC₁₋₆ alkyl may be optionally substituted with C(O)NR^{c}R^{d} where R^{c} and R^{d} are both hydrogen; —C(O)O C₁₋₆ alkyl, eg methoxycarbonyl, ethoxycarbonyl or isopropoxycarbonyl; and —C(O)OC₁₋₆ alkylC₃₋₈ cycloalkyl eg cyclopropylmethoxycarbonyl.

Most preferably R⁹ is selected from: chloro; methyl; difluoromethyl; amino; methylamino; (2-cyanoethyl)amino; isobutylamino; (2-fluoroethyl)amino; (2-fluoro-2-methyl-propyl)amino; carbamoylmethylamino; (1,2,4-triazol-1yl)ethylamino; [3-(methylthio)propyl]amino; (cyclopropylmethyl)amino; (methyl)(cyclopropylmethyl]amino; {[1-(aminocarbonyl)cyclopropyl]methyl}amino; (methoxycarbonyl)amino; (ethoxycarbonyl)amino; (isopropoxycarbonyl)amino; (methyl)(ethoxycarbonyl)amino; and [(cyclopropylmethoxy)carbonyl]amino.

Preferably X is CR¹⁰. Suitable compounds include those where, when R¹⁰ is halo, preferred halo substituents are fluoro, chloro or bromo. Further suitable compounds include those where, when R¹⁰ is selected from C₁₋₆ alkyl or C₁₋₆ alkoxy where the C₁₋₆ alkyl or C₁₋₆ alkoxy are optionally substituted with one or more halo substituents, preferred halo substituents are fluoro, chloro or bromo. Preferably R¹⁰ is selected from chloro, or fluoro. Most preferably R¹⁰ is chloro. Other preferred compounds are those in which R⁷and R¹⁰ are the same. More preferably, both R⁷ and R¹⁰ are Cl.

A further group of suitable compounds of the present invention are those of formula (I) where:
R¹, R³—R¹¹, X, R^{c}, R^{d}, n, and het are all as defined for formula (I) above; and
R² is selected from cyano, hydroxy, C(O)OH, het, S(O)ₙR¹¹, C(O)NR^{a}R^{b} and C(S)NR^{a}R^{b};
or R² is selected from C₁₋₆ alkanoyl, C(O)OC₁₋₆ alkyl, and amino, each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylCl,₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
where R^{a} and R^{b} are independently selected from hydrogen, het, phenyl, and S(O)ₙR¹¹; or either one or both of R^{a} and R^{b} are independently selected from C₁₋₆ alkyl, C₃₋₈ cycloalkyl, C₃₋₈ cycloalkylC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹; or R^{a} and R^{b} together with the N atom to which they are attached may form a three to seven-membered saturated, partially saturated, or unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or a pharmaceutically acceptable salt or a prodrug thereof.

Preferably, in these compounds of formula (I): R¹ is selected from CF₃, OCF₃, or SF₅; both R³ and R⁴ are the same as each other and are selected from: hydrogen; fluoro; and chloro and both R⁵ and R⁶ are hydrogen; R⁷ is chloro; R⁸ is cyano; and X is CR¹⁰ where R¹⁰ is chloro.

A yet further group of suitable compounds of the present invention are those of formula (I) where:
R¹—R⁸, X, R^{c}, R^{d}, n, R¹⁰—R¹¹, and het are all as defined for formula (I) above; and
R⁹ is selected from hydrogen, halo, and S(O)ₙR¹¹;
or R⁹ is selected from C₁₋₆ alkyl, C₃₋₈ cycloalkylC₁₋₆ alkoxy, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁹ is NR^{e}R^{f} where R^{e} and R^{f} are independently selected from hydrogen; or either one or both of R^{e} and R^{f} are independently selected from C₁₋₆ alkyl, C₃₋₈ cycloalkylC₁₋₆ alkyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or a pharmaceutically acceptable salt or a prodrug thereof.

Preferably, in these compounds of formula (I): R¹ is selected from CF₃, OCF₃, or SF₅; both R³ and R⁴ are the same as each other and are selected from: hydrogen; fluoro; and chloro and both R⁵ and R⁶ are hydrogen; R⁷is chloro; R⁸ is cyano; and X is CR¹⁰ where R¹⁰ is chloro.

A still further of suitable compounds of the present invention are those of formula (I) where:
R¹, R³—R⁸, X, R^{c}, R^{d}, n, R¹⁰—R¹¹, and het are all as defined for formula (I) above;
R² is selected from cyano, hydroxy, C(O)OH, het, S(O)ₙR¹¹, C(O)NR^{a}R^{b} and C(S)NR^{a}R^{b};
or R² is selected from C₁₋₆ alkanoyl, C(O)OC₁₋₆ alkyl, and amino, each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
where R^{a} and R^{b} are independently selected from hydrogen, het, phenyl, and S(O)ₙR¹¹; or either one or both of R^{a} and R^{b} are independently selected from C₁₋₆ alkyl, C₃₋₈ cycloalkyl, C₃₋₈ cycloalkylC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹; or R^{a} and R^{b} together with the N atom to which they are attached may form a three to seven-membered saturated, partially saturated, or unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R⁹ is selected from hydrogen, halo, and S(O)ₙR¹¹;
or R⁹ is selected from C₁₋₆ alkyl, C₃₋₈ cycloalkylC₁₋₆ alkoxy, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁹ is NR^{e}R^{f} where R^{e} and R^{f} are independently selected from hydrogen; or either one or both of R^{e} and R^{f} are independently selected from C₁₋₆ alkyl, C₃₋₈ cycloalkylC₁₋₆ alkyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or a pharmaceutically acceptable salt or a prodrug thereof.

Preferably, in these compounds of formula (I): R¹ is selected from CF₃, OCF₃, or SF₅; both R³ and R⁴ are the same as each other and are selected from: hydrogen; fluoro; and chloro and both R⁵ and R⁶ are hydrogen; R⁷ is chloro; R⁸ is cyano; and X is CR¹⁰ where R¹⁰ is chloro.

An even further group of suitable compounds of formula (I) are those wherein:
R¹—R², R⁷—R⁹, X, R^{c}, R^{d}, n, R¹¹ and het are all as defined for formula (I) above; and
R³, R⁴, R⁵ and R⁶ are independently selected from hydrogen, halo, cyano, hydroxy, C(O)OH, nitro, phenyl, and S(O)ₙR¹¹;
or either one or more of R³, R⁴, R⁵ and R⁶ are independently selected from C₁₋₄ alkyl, C(O)NR^{c}R^{d}, C(S)NR^{c}R^{d}, C₁₋₄ alkoxy, C₁₋₄ alkanoyl, C(O)OC₁₋₄ alkyl, amino which R³, R⁴, R⁵ and R⁶ may be optionally and independently further substituted by one or more substituents selected from, where chemically possible, cyano, nitro, halo, hydroxy, C₁₋₄ alkyl and amino;
and where not more than two of R³, R⁴, R⁵ and R⁶ are selected from cyano, hydroxy, C(O)OH, nitro, phenyl, S(O)ₙR¹¹, C(O)NR^{c}R^{d}, C(S)NR^{c}R^{d}, C₁₋₄ alkoxy, C₁₋₄ alkanoyl, C(O)OC₁₋₄ alkyl, and amino;
or a pharmaceutically acceptable salt or a prodrug thereof.

Preferably, in these compounds of formula (I): R¹ is selected from CF₃, OCF₃, or SF₅; R⁷ is chloro; R⁸ is cyano; and X is CR¹⁰ where R¹⁰ is chloro.

Preferred individual compounds of formula (I) are selected from:
5-amino-1-[2,6-dichloro-4-pentafluorothiophenyl]-4-[1-(methylsulfonyl)cyclopropyl]-1H-pyrazole-3-carbonitrile;
1-{5-amino-3-cyano-1-[2,6-dichloro-4-(trifluoromethyl)phenyl]-1H-pyrazol-4-yl}cyclopropanecarboxamide;
methyl 1-{5-amino-3-cyano-1-[2,6-dichloro-4-(trifluoromethyl)phenyl]-1H-pyrazol-4-yl}cyclopropane-carboxylate;
5-amino-1-[2,6-dichloro-4-(trifluoromethyl)phenyl]-4-[2,2-difluoro-1-(methylsulfonyl)cyclopropyl]-1H-pyrazole-3-carbonitrile;
1-{5-amino-3-cyano-1-[2,6-dichloro-4-(trifluoromethyl)phenyl]-1H-pyrazol-4-yl}-N,N-dimethylcyclopropanecarboxamide;
5-amino-1-[2,6-dichloro-4-(trifluoromethyl)phenyl]-4-[1-(methylsulfonyl)cyclopropyl]-1H-pyrazole-3-carbonitrile;
5-amino-4-(1-amino-2,2-difluorocyclopropyl)-1-[2,6-dichloro-4-(trifluoromethyl)phenyl]-1H-pyrazole-3-carbonitrile;
1-{5-amino-3-cyano-1-[2,6-dichloro-4-(trifluoromethyl)phenyl]-1H-pyrazol-4-yl}-2,2-difluoro-N,N-dimethyl-cyclopropanesulfonamide;
5-amino-1-[2,6-dichloro-4-(trifluoromethyl)phenyl]-4-[1-(pyrrolidin-1-ylcarbonyl)cyclopropyl]-1H-pyrazole-3-carbonitrile;
5-amino-4-(1-cyanocyclopropyl)-1-[2,6-dichloro-4-(trifluoromethyl)phenyl]-1H-pyrazole-3-carbonitrile;
5-amino-4-(1-cyanocyclopropyl)-1-[2,6-dichloro-4-pentafluorothiophenyl]-1H-pyrazole-3-carbonitrile;
1-{5-amino-3-cyano-1-[2,6-dichloro-4-(trifluoromethyl)phenyl]-1H-pyrazol-4-yl}-2,2-difluorocyclopropanesulfonamide;
1-{5-amino-3-cyano-1-[2,6-dichloro-4-pentafluorothiophenyl]-1H-pyrazol-4-yl}cyclopropanecarboxamide;
1-{3-cyano-1-[2,6-dichloro-4-pentafluorothiophenyl]-5-(isobutylamino)-1H-pyrazol-4-yl}cyclopropanecarboxamide;
1-{5-amino-3-cyano-1-[2,6-dichloro-4-pentafluorothiophenyl]-1H-pyrazol-4-yl}-N-isopropylcyclopropanecarboxamide;
1-{3-cyano-1-[2,6-dichloro-4-pentafluorothiophenyl]-5-[(2-fluoroethyl)amino]-1H-pyrazol-4-yl}cyclopropane-carboxamide;
1-{5-[(2-amino-2-oxoethyl)amino]-3-cyano-1-[2,6-dichloro-4-pentafluorothiophenyl]-1H-pyrazol-4-yl}-cyclopropanecarboxamide;
1-{5-amino-3-cyano-1-[2,6-dichloro-4-pentafluorothiophenyl]-1H-pyrazol-4-yl}-2,2-dichlorocyclopropane-carboxamide;
1-{3-cyano-5-[(cyclopropylmethyl)amino]-1-[2,6-dichloro-4-pentafluorothiophenyl]-1H-pyrazol-4-yl}-N-(pyridin-4-ylmethyl)cyclopropanecarboxamide;
isopropyl {4-[1-(aminocarbonyl)cyclopropyl]-3-cyano-1-[2,6-dichloro-4-pentafluorothiophenyl]-1H-pyrazol-5-yl}carbamate;
1-(3-cyano-1-[2,6-dichloro-4-pentafluorothiophenyl]-5-{[2-(1H-1,2,4-triazol-1-yl)ethyl]amino}-1H-pyrazol-4-yl)cyclopropanecarboxamide;
1-{3-cyano-5-[(2-cyanoethyl)amino]-1-[2,6-dichloro-4-pentafluorothiophenyl]-1H-pyrazol-4-yl}cyclopropane-carboxamide;
1-(5-amino-3-cyano-1-{2,6-dichloro-4-[1,2,2,2-tetrafluoro-1-(trifluoromethyl)ethyl]phenyl}-1H-pyrazol-4-yl)cyclopropanecarboxamide;
1-(3-cyano-1-[2,6-dichloro-4-pentafluorothiophenyl]-5-{[3-(methylthio)propyl]amino}-1H-pyrazol-4-yl)-cyclopropanecarboxamide;
1-{3-cyano-5-[(cyclopropylmethyl)amino]-1-[2,6-dichloro-4-pentafluorothiophenyl]-1H-pyrazol-4-yl}-N-[(5-methyl-4H-1,2,4-triazol-3-yl)methyl]cyclopropanecarboxamide;
1-{3-cyano-5-[(cyclopropylmethyl)(methyl)amino]-1-[2,6-dichloro-4-pentafluorothiophenyl]-1H-pyrazol-4-yl}cyclopropanecarboxamide;
[1-(fluoromethyl)cyclopropyl]methyl{4-[1-(aminocarbonyl)cyclopropyl]-3-cyano-1-[2,6-dichloro-4-pentafluorothiophenyl]-1H-pyrazol-5-yl}carbamate;
1-{3-cyano-1-[2,6-dichloro-4-pentafluorothiophenyl]-5-(methylamino)-1H-pyrazol-4-yl}-2,2-difluorocyclopropanecarboxamide;
1-{3-cyano-1-[2,6-dichloro-4-pentafluorothiophenyl]-5-[(3,3,3-trifluoropropyl)amino]-1H-pyrazol-4-yl}cyclopropanecarboxamide;
1-(5-{[(2-chloro-1,3-thiazol-5-yl)methyl]amino}-3-cyano-1-[2,6-dichloro-4-pentafluorothiophenyl]-1H-pyrazol-4-yl)cyclopropanecarboxamide;
1-{3-cyano-1-[2,6-dichloro-4-pentafluorothiophenyl]-5-[(isoxazol-5-ylmethyl)amino]-1H-pyrazol-4-yl}cyclopropanecarboxamide;
N˜3˜-{4-[1-(aminocarbonyl)cyclopropyl]-3-cyano-1-[2,6-dichloro-4-pentafluorothiophenyl]-1H-pyrazol-5-yl}-beta-alaninamide;
1-{3-cyano-1-[2,6-dichloro-4-pentafluorothiophenyl]-5-[(5,5,5-trifluoropentyl)amino]-1H-pyrazol-4-yl}cyclopropanecarboxamide;
1-{3-cyano-1-[2,6-dichloro-4-pentafluorothiophenyl]-5-(propylamino)-1H-pyrazol-4-yl}cyclopropanecarboxamide;
1-{3-cyano-5-[(cyclobutylmethyl)amino]-1-[2,6-dichloro-4-pentafluorothiophenyl]-1H-pyrazol-4-yl}cyclopropanecarboxamide;
1-{3-cyano-1-[2,6-dichloro-4-pentafluorothiophenyl]-5-(dimethylamino)-1H-pyrazol-4-yl}cyclopropanecarboxamide;
ethyl {4-[1-(aminocarbonyl)cyclopropyl]-3-cyano-1-[2,6-dichloro-4-(trifluoromethoxy)phenyl]-1H-pyrazol-5-yl}carbamate;
2,2-dichloro-1-{3-cyano-1-[2,6-dichloro-4-pentafluorothiophenyl]-5-(methylamino)-1H-pyrazol-4-yl}cyclopropanecarboxamide;
1-{5-amino-3-cyano-1-[2,6-dichloro-4-(trifluoromethoxy)phenyl]-1H-pyrazol-4-yl}-2,2-dichlorocyclopropane-carboxamide;
1-{3-cyano-5-({2-[(cyclopropylmethyl)amino]-2-oxoethyl}amino)-1-[2,6-dichloro-4-pentafluorothiophenyl]-1H-pyrazol-4-yl}cyclopropanecarboxamide;
1-{5-[(4-amino-4-oxobutyl)amino]-3-cyano-1-[2,6-dichloro-4-pentafluorothiophenyl]-1H-pyrazol-4-yl}cyclopropanecarboxamide;
1-{3-cyano-1-[2,6-dichloro-4-pentafluorothiophenyl]-5-[(1,3-thiazol-2-ylmethyl)amino]-1H-pyrazol-4-yl}-cyclopropanecarboxamide;
1-{3-cyano-5-[(cyclopropylmethyl)amino]-1-[2,6-dichloro-4-pentafluorothiophenyl]-1H-pyrazol-4-yl}-N-(2-methoxyethyl)cyclopropanecarboxamide;
1-{3-cyano-5-[(cyclopropylmethyl)amino]-1-[2,6-dichloro-4-pentafluorothiophenyl]-1H-pyrazol-4-yl}-N-(2-hydroxyethyl)cyclopropanecarboxamide;
1-{3-cyano-5-[(cyclopropylmethyl)amino]-1-[2,6-dichloro-4-pentafluorothiophenyl]-1H-pyrazol-4-yl}-N-(pyridin-2-ylmethyl)cyclopropanecarboxamide;
1-{3-cyano-5-[(cyclopropylmethyl)amino]-1-[2,6-dichloro-4-pentafluorothiophenyl]-1H-pyrazol-4-yl}-N-(pyridin-3-yl methyl)cyclopropanecarboxamide;
1-{3-cyano-5-[(cyclopropylmethyl)amino]-1-[2,6-dichloro-4-pentafluorothiophenyl]-1H-pyrazol-4-yl}-N-(2-hydroxy-2-methylpropyl)cyclopropanecarboxamide;
1-(3-cyano-1-[2,6-dichloro-4-pentafluorothiophenyl]-5-{[2-(1-methyl-1H-pyrazol-4-yl)ethyl]amino}-1H-pyrazol-4-yl)cyclopropanecarboxamide;
1-{3-cyano-1-[2,6-dichloro-4-pentafluorothiophenyl]-5-(dimethylamino)-1H-pyrazol-4-yl}-2,2-difluorocyclopropanecarboxamide;
1-{3-cyano-1-[2,6-dichloro-4-pentafluorothiophenyl]-5-(methylthio)-1H-pyrazol-4-yl}cyclopropanecarboxamide;
1-{3-cyano-1-[2,6-dichloro-4-pentafluorothiophenyl]-5-[(2-methoxyethyl)(methyl)amino]-1H-pyrazol-4-yl}cyclopropanecarboxamide;
1-(5-{[(5-chloro-1,3-dimethyl-1H-pyrazol-4-yl)methyl]amino}-3-cyano-1-[2,6-dichloro-4-pentafluorothiophenyl]-1H-pyrazol-4-yl)cyclopropanecarboxamide;
1-{5-amino-3-cyano-1-[2,6-dichloro-4-pentafluorothiophenyl]-1H-pyrazol-4-yl}-2,2-difluorocyclopropanecarboxamide;
1-{5-amino-3-cyano-1-[2,6-dichloro-4-(trifluoromethoxy)phenyl]-1H-pyrazol-4-yl}cyclopropanecarboxamide;
1-{5-amino-3-cyano-1-[2,6-dichloro-4-(trifluoromethyl)phenyl]-1H-pyrazol-4-yl}-2,2-difluorocyclopropane-carboxamide;
1-{5-amino-3-cyano-1-[2,6-dichloro-4-pentafluorothiophenyl]-1H-pyrazol-4-yl}-N-methylcyclopropane-carboxamide;
1-{5-amino-3-cyano-1-[2,6-dichloro-4-(trifluoromethyl)phenyl]-1H-pyrazol-4-yl}-N-cyclopropylcyclopropane-carboxamide;
1-{5-amino-3-cyano-1-[2,6-dichloro-4-(trifluoromethyl)phenyl]-1H-pyrazol-4-yl}-N-(cyclopropylmethyl)-cyclopropanecarboxamide;
1-{5-amino-3-cyano-1-[2,6-dichloro-4-(trifluoromethyl)phenyl]-1H-pyrazol-4-yl}-N-pyridin-2-ylcyclopropane-carboxamide;
1-{5-amino-1-[2,6-dichloro-4-(trifluoromethyl)phenyl]-3-(trifluoromethyl)-1H-pyrazol-4-yl}cyclopropane-carboxamide;
1-(3-cyano-1-[2,6-dichloro-4-(trifluoromethyl)phenyl]-5-{[(l E)-(dimethylamino)methylene]amino}-1H-pyrazol-4-yl)cyclopropanecarboxamide;
1-{5-amino-3-cyano-1-[2,6-dichloro-4-pentafluorothiophenyl]-1H-pyrazol-4-yl}-N-(2,2,2-trifluoroethyl)-cyclopropanecarboxamide;
1-{3-cyano-1-[2,6-dichloro-4-(trifluoromethoxy)phenyl]-5-(methylamino)-1H-pyrazol-4-yl}-2,2-difluoro-cyclopropanecarboxamide;
1-{3-cyano-1-[2,6-dichloro-4-(trifluoromethoxy)phenyl]-5-(methylamino)-1H-pyrazol-4-yl}cyclopropane-carboxamide;
1-{3-cyano-5-[(cyclopropylmethyl)amino]-1-[2,6-dichloro-4-(trifluoromethyl)phenyl]-1H-pyrazol-4-yl}-N-methylcyclopropanecarboxamide;
1-{5-amino-3-cyano-1-[2,6-dichloro-4-(trifluoromethyl)phenyl]-1H-pyrazol-4-yl}-2,2-dimethylcyclopropane-carboxamide;
1-{3-cyano-1-[2,6-dichloro-4-pentafluorothiophenyl]-5-[(4H-1,2,4-triazol-3-ylmethyl)amino]-1H-pyrazol-4-yl}cyclopropanecarboxamide;
1-(3-cyano-1-[2,6-dichloro-4-pentafluorothiophenyl]-5-{[(1-methylcyclopropyl)methyl]amino}-1H-pyrazol-4-yl)cyclopropanecarboxamide;
1-{3-cyano-1-[2,6-dichloro-4-pentafluorothiophenyl]-5-({4-[(methylamino)sulfonyl]benzyl}amino)-1H-pyrazol-4-yl}cyclopropanecarboxamide;
1-{3-cyano-1-[2,6-dichloro-4-pentafluorothiophenyl]-5-({4-[(methylsulfonyl)amino]benzyl}amino)-1H-pyrazol-4-yl}cyclopropanecarboxamide;
1-{3-cyano-1-[2,6-dichloro-4-pentafluorothiophenyl]-5-[(tetrahydro-2H-pyran-4-ylmethyl)amino]-1H-pyrazol-4-yl}cyclopropanecarboxamide;
1-{3-cyano-5-[(cyclopropylmethyl)amino]-1-[2,6-dichloro-4-pentafluorothiophenyl]-1H-pyrazol-4-yl}-N-(3-isopropoxypropyl)cyclopropanecarboxamide;
1-{3-cyano-1-[2,6-dichloro-4-pentafluorothiophenyl]-5-({2-oxo-2-[(2,2,2-trifluoroethyl)amino]ethyl}amino)-1H-pyrazol-4-yl}cyclopropanecarboxamide;
5-amino-1-[2,6-dichloro-4-(trifluoromethyl)phenyl]-4-[2,2-difluoro-1-(methylthio)cyclopropyl]-1H-pyrazole-3-carbonitrile;
S-methyl 5-amino-1-[2,6-dichloro-4-(trifluoromethyl)phenyl]-4-[2,2-difluoro-1-(methylthio)cyclopropyl]-1H-pyrazole-3-carbothioate;
1-{3-cyano-5-[(cyclopropylmethyl)amino]-1-[2,6-dichloro-4-(trifluoromethyl)phenyl]-1H-pyrazol-4-yl}cyclopropanecarboxamide;
1-{5-(benzylamino)-3-cyano-1-[2,6-dichloro-4-(trifluoromethyl)phenyl]-1H-pyrazol-4-yl}cyclopropanecarboxamide;
1-{3-cyano-1-[2,6-dichloro-4-(trifluoromethyl)phenyl]-5-[(pyridin-2-ylmethyl)amino]-1H-pyrazol-4-yl}cyclopropanecarboxamide;
1-{3-cyano-1-[2,6-dichloro-4-(trifluoromethyl)phenyl]-5-[(2,2-dimethylpropyl)amino]-1H-pyrazol-4-yl}cyclopropanecarboxamide;
1-(3-cyano-1-[2,6-dichloro-4-(trifluoromethyl)phenyl]-5-{[4-(methylsulfonyl)benzyl]amino}-1H-pyrazol-4-yl)cyclopropanecarboxamide;
1-{3-cyano-1-[2,6-dichloro-4-(trifluoromethyl)phenyl]-5-[(pyridin-4-ylmethyl)amino]-1H-pyrazol-4-yl}cyclopropanecarboxamide;
1-{3-cyano-1-[2,6-dichloro-4-(trifluoromethyl)phenyl]-5-[(2,2,2-trifluoroethyl)amino]-1H-pyrazol-4-yl}cyclopropanecarboxamide;
1-{3-cyano-1-[2,6-dichloro-4-(trifluoromethyl)phenyl]-5-[(1H-imidazol-2-ylmethyl)amino]-1H-pyrazol-4-yl}cyclopropanecarboxamide;
1-{3-cyano-1-[2,6-dichloro-4-pentafluorothiophenyl]-1H-pyrazol-4-yl}-2,2-difluorocyclopropanecarboxamide;
1-{3-cyano-1-[2,6-dichloro-4-pentafluorothiophenyl]-1H-pyrazol-4-yl}cyclopropanecarboxamide;
1-{5-chloro-3-cyano-1-[2,6-dichloro-4-pentafluorothiophenyl]-1H-pyrazol-4-yl}cyclopropanecarboxamide;
1-{5-chloro-3-cyano-1-[2,6-dichloro-4-pentafluorothiophenyl]-1H-pyrazol-4-yl}-2,2-difluorocyclopropane-carboxamide;
5-amino-1-[2,6-dichloro-4-(trifluoromethyl)phenyl]-4-[1-(5-methyl-1,3,4-oxadiazol-2-yl)cyclopropyl]-1H-pyrazole-3-carbonitrile;
1-{5-amino-3-cyano-1-[2,6-dichloro-4-(trifluoromethyl)phenyl]-1H-pyrazol-4-yl}-2,2-dimethylcyclopropane-carboxylic acid;
1-{3-cyano-1-[2,6-dichloro-4-pentafluorothiophenyl]-5-(methylamino)-1H-pyrazol-4-yl}cyclopropane-carboxamide;
1-{3-cyano-1-[2,6-dichloro-4-pentafluorothiophenyl]-5-(difluoromethyl)-1H-pyrazol-4-yl}cyclopropane-carboxamide;
cyclopropylmethyl {4-[1-(aminocarbonyl)cyclopropyl]-3-cyano-1-[2,6-dichloro-4-pentafluorothiophenyl]-1H-pyrazol-5-yl}carbamate;
ethyl {4-[1-(aminocarbonyl)cyclopropyl]-3-cyano-1-[2,6-dichloro-4-pentafluorothiophenyl]-1H-pyrazol-5-yl}methylcarbamate;
1-[({4-[1-(aminocarbonyl)cyclopropyl]-3-cyano-1-[2,6-dichloro-4-pentafluorothiophenyl]-1H-pyrazol-5-yl}amino)methyl]cyclopropanecarboxamide;
1-{3-cyano-1-[2,6-dichloro-4-pentafluorothiophenyl]-5-methyl-1H-pyrazol-4-yl}cyclopropanecarboxamide;
1-{3-cyano-5-[(cyclopropylmethyl)amino]-1-[2,6-dichloro-4-(trifluoromethoxy)phenyl]-1H-pyrazol-4-yl}-cyclopropanecarboxamide;
1-{3-cyano-1-[2,6-dichloro-4-pentafluorothiophenyl]-5-[(2-fluoro-2-methylpropyl)amino]-1H-pyrazol-4-yl}cyclopropanecarboxamide;
methyl {4-[1-(aminocarbonyl)cyclopropyl]-3-cyano-1-[2,6-dichloro-4-pentafluorothiophenyl]-1H-pyrazol-5-yl}carbamate;
1-{5-amino-3-cyano-1-[2,6-dichloro-4-(trifluoromethoxy)phenyl]-1H-pyrazol-4-yl}-2,2-difluorocyclopropane-carboxamide;
ethyl {4-[1-(aminocarbonyl)cyclopropyl]-3-cyano-1-[2,6-dichloro-4-pentafluorothiophenyl]-1H-pyrazol-5-yl}carbamate;
cyclopropylmethyl {4-[1-(aminocarbonyl)cyclopropyl]-3-cyano-1-[2,6-dichloro-4-pentafluorothiophenyl]-1H-pyrazol-5-yl}methylcarbamate;
1-{3-cyano-1-[2,6-dichloro-4-pentafluorothiophenyl]-5-[(4,4,4-trifluorobutyl)amino]-1H-pyrazol-4-yl}-cyclopropanecarboxamide;
1-{3-cyano-1-[2,6-dichloro-4-pentafluorothiophenyl]-5-(ethylamino)-1H-pyrazol-4-yl}cyclopropanecarboxamide;
tert-butyl {1-[({4-[1-(aminocarbonyl)cyclopropyl]-3-cyano-1-[2,6-dichloro-4-pentafluorothiophenyl]-1H-pyrazol-5-yl}amino)methyl]cyclopropyl}carbamate;
1-(3-cyano-1-[2,6-dichloro-4-(trifluoromethyl)phenyl]-5-{[4-(trifluoromethyl)benzyl]amino}-1H-pyrazol-4-yl)cyclopropanecarboxamide;
1-{3-cyano-5-(cyclopropylmethoxy)-1-[2,6-dichloro-4-pentafluorothiophenyl]-1H-pyrazol-4-yl}cyclopropane-carboxamide;
1-{3-cyano-1-[2,6-dichloro-4-pentafluorothiophenyl]-5-[(2-isopropoxyethyl)amino]-1H-pyrazol-4-yl}cyclopropanecarboxamide;
1-{3-cyano-1-[2,6-dichloro-4-pentafluorothiophenyl]-5-vinyl-1H-pyrazol-4-yl}cyclopropanecarboxamide;
cyclobutyl {4-[1-(aminocarbonyl)cyclopropyl]-3-cyano-1-[2,6-dichloro-4-pentafluorothiophenyl]-1H-pyrazol-5-yl}carbamate;
1-[5-amino-3-cyano-1-(2,6-dichloro-4-cyanophenyl)-1H-pyrazol-4-yl]cyclopropanecarboxamide;
1-{3-cyano-1-[2,6-dichloro-4-pentafluorothiophenyl]-5-[(4-fluorobenzyl)amino]-1H-pyrazol-4-yl}cyclopropane-carboxamide;
1-{3-cyano-1-[2,6-dichloro-4-pentafluorothiophenyl]-5-(methoxymethyl)-1H-pyrazol-4-yl}cyclopropane-carboxamide;
ethyl {4-[1-(aminocarbonyl)cyclopropyl]-3-cyano-1-[2,6-dichloro-4-(trifluoromethyl)phenyl]-1H-pyrazol-5-yl}carbamate;
1-{3-cyano-5-[(cyclopropylmethyl)amino]-1-[2,6-dichloro-4-pentafluorothiophenyl]-1H-pyrazol-4-yl}cyclopropanecarboxamide;
methyl 1-{5-(benzylamino)-3-cyano-1-[2,6-dichloro-4-(trifluoromethyl)phenyl]-1H-pyrazol-4-yl}cyclopropanecarboxylate;
1-{3-cyano-5-[(cyclopropylmethyl)amino]-1-[2,6-dichloro-4-pentafluorothiophenyl]-1H-pyrazol-4-yl}-2,2-difluorocyclopropanecarboxamide;
1-{3-cyano-5-[(cyclopropylmethyl)amino]-1-[2,6-dichloro-4-(trifluoromethyl)phenyl]-1H-pyrazol-4-yl}-2,2-difluorocyclopropanecarboxamide;
4-(1-cyanocyclopropyl)-1-[2,6-dichloro-4-pentafluorothiophenyl]-5-(methylamino)-1H-pyrazole-3-carbonitrile;
1-{5-amino-3-cyano-1-[2,6-dichloro-4-(trifluoromethyl)phenyl]-1H-pyrazol-4-yl}cyclopropanecarbothioamide;
5-amino-1-[2,6-dichloro-4-(trifluoromethyl)phenyl]-4-[1-(1,3-thiazol-2-yl)cyclopropyl]-1H-pyrazole-3-carbonitrile;
1-(3-cyano-1-[2,6-dichloro-4-(trifluoromethyl)phenyl]-5-{[(1-oxidopyridin-4-yl)methyl]amino}-1H-pyrazol-4-yl)cyclopropanecarboxamide;
1-{5-amino-3-cyano-1-[2,6-dichloro-4-pentafluorothiophenyl]-1H-pyrazol-4-yl}-N-(methylsulfonyl)-cyclopropanecarboxamide;
1-{3-cyano-5-[(2-cyclopropylethyl)amino]-1-[2,6-dichloro-4-pentafluorothiophenyl]-1H-pyrazol-4-yl}cyclopropanecarboxamide;
1-[2,6-dichloro-4-pentafluorothiophenyl]-7-methyl-5-oxo-5,6,7,8-tetrahydro-1H-spiro[cyclopropane-1,4-pyrazolo[3,4-d][1,3]diazepine]-3-carbonitrile;
5-amino-1-[2,6-dichloro-4-(trifluoromethyl)phenyl]-4-[2,2-difluoro-1-(methylsulfinyl)cyclopropyl]-1H-pyrazole-3-carbonitrile;
5-amino-1-[2,6-dichloro-4-(trifluoromethyl)phenyl]-4-[2,2-difluoro-1-(methylsulfinyl)cyclopropyl]-1H-pyrazole-3-carbonitrile;
1-{3-cyano-1-[2,6-dichloro-4-pentafluorothiophenyl]-5-(isopropylamino)-1H-pyrazol-4-yl}cyclopropane-carboxamide;
1-{3-cyano-1-[2,6-dichloro-4-(trifluoromethoxy)phenyl]-5-(isopropylamino)-1H-pyrazol-4-yl}-2,2-difluorocyclopropanecarboxamide;
4-(1-cyanocyclopropyl)-5-[(cyclopropylmethyl)amino]-1-[2,6-dichloro-4-pentafluorothiophenyl]-1H-pyrazole-3-carbonitrile;
1-{3-cyano-1-[2,6-dichloro-4-pentafluorothiophenyl]-5-[({1-[(methylsulfonyl)amino]cyclopropyl}methyl)amino]-1H-pyrazol-4-yl}cyclopropanecarboxamide;
1-(5-{[(1-aminocyclopropyl)methyl]amino}-3-cyano-1-[2,6-dichloro-4-pentafluorothiophenyl]-1H-pyrazol-4-yl)cyclopropanecarboxamide;
1-{3-cyano-1-[2,6-dichloro-4-pentafluorothiophenyl]-5-(methylsulfinyl)-1H-pyrazol-4-yl}cyclopropanecarboxamide;
1-{3-cyano-1-[2,6-dichloro-4-pentafluorothiophenyl]-5-(methylsulfonyl)-1H-pyrazol-4-yl}cyclopropanecarboxamide;
4-({4-[1-(aminocarbonyl)cyclopropyl]-3-cyano-1-[2,6-dichloro-4-pentafluorothiophenyl]-1H-pyrazol-5-yl}amino)butanoic acid;
or a pharmaceutically acceptable salt or prodrug thereof.

Even more preferred individual compounds of the present invention are selected from:
1-{3-cyano-5-[(cyclopropylmethyl)amino]-1-[2,6-dichloro-4-pentafluorothiophenyl]-1H-pyrazol-4-yl}cyclopropanecarboxamide;
cyclopropylmethyl {4-[1-(aminocarbonyl)cyclopropyl]-3-cyano-1-[2,6-dichloro-4-pentafluorothiophenyl]-1H-pyrazol-5-yl}carbamate;
1-{5-amino-3-cyano-1-[2,6-dichloro-4-pentafluorothiophenyl]-1H-pyrazol-4-yl}cyclopropanecarboxamide;
1-{3-cyano-1-[2,6-dichloro-4-pentafluorothiophenyl]-5-(methylamino)-1H-pyrazol-4-yl}cyclopropanecarboxamide;
1-{3-cyano-5-[(cyclopropylmethyl)amino]-1-[2,6-dichloro-4-(trifluoromethoxy)phenyl]-1H-pyrazol-4-yl}cyclopropanecarboxamide;
1-{5-amino-3-cyano-1-[2,6-dichloro-4-pentafluorothiophenyl]-1H-pyrazol-4-yl}-2,2-dichlorocyclopropane-carboxamide;
1-{5-amino-3-cyano-1-[2,6-dichloro-4-(trifluoromethoxy)phenyl]-1H-pyrazol-4-yl}-2,2-difluorocyclopropane-carboxamide;
1-{3-cyano-1-[2,6-dichloro-4-pentafluorothiophenyl]-5-[(2-fluoroethyl)amino]-1H-pyrazol-4-yl}cyclopropane-carboxamide;
1-{3-cyano-1-[2,6-dichloro-4-pentafluorothiophenyl]-5-(methylamino)-1H-pyrazol-4-yl}-2,2-difluorocyclopropanecarboxamide;
1-[({4-[1-(aminocarbonyl)cyclopropyl]-3-cyano-1-[2,6-dichloro-4-pentafluorothiophenyl]-1H-pyrazol-5-yl}amino)methyl]cyclopropanecarboxamide;
ethyl {4-[1-(aminocarbonyl)cyclopropyl]-3-cyano-1-[2,6-dichloro-4-pentafluorothiophenyl]-1H-pyrazol-5-yl}methylcarbamate;
1-{3-cyano-1-[2,6-dichloro-4-pentafluorothiophenyl]-5-(isobutylamino)-1H-pyrazol-4-yl}cyclopropane-carboxamide;
1-{3-cyano-5-[(cyclopropylmethyl)amino]-1-[2,6-dichloro-4-pentafluorothiophenyl]-1H-pyrazol-4-yl}-N-(pyridin-4-ylmethyl)cyclopropanecarboxamide;
isopropyl {4-[1-(aminocarbonyl)cyclopropyl]-3-cyano-1-[2,6-dichloro-4-pentafluorothiophenyl]-1H-pyrazol-5-yl}carbamate;
1-(3-cyano-1-[2,6-dichloro-4-pentafluorothiophenyl]-5-{[3-(methylthio)propyl]amino}-1H-pyrazol-4-yl)-cyclopropanecarboxamide;
1-{3-cyano-1-[2,6-dichloro-4-pentafluorothiophenyl]-5-[(2-fluoro-2-methylpropyl)amino]-1H-pyrazol-4-yl}cyclopropanecarboxamide;
1-(3-cyano-1-[2,6-dichloro-4-pentafluorothiophenyl]-5-{[2-(1H-1,2,4-triazol-1-yl)ethyl]amino}-1H-pyrazol-4-yl)cyclopropanecarboxamide;
1-{3-cyano-5-[(2-cyanoethyl)amino]-1-[2,6-dichloro-4-pentafluorothiophenyl]-1H-pyrazol-4-yl}cyclopropane-carboxamide;
1-{5-chloro-3-cyano-1-[2,6-dichloro-4-pentafluorothiophenyl]-1H-pyrazol-4-yl}-2,2-difluorocyclopropane-carboxamide;
1-{5-chloro-3-cyano-1-[2,6-dichloro-4-pentafluorothiophenyl]-1H-pyrazol-4-yl}cyclopropanecarboxamide;
1-{3-cyano-1-[2,6-dichloro-4-pentafluorothiophenyl]-5-methyl-1H-pyrazol-4-yl}cyclopropanecarboxamide;
1-{3-cyano-1-[2,6-dichloro-4-pentafluorothiophenyl]-5-(difluoromethyl)-1H-pyrazol-4-yl}cyclopropanecarboxamide;
1-{5-amino-3-cyano-1-[2,6-dichloro-4-pentafluorothiophenyl]-1H-pyrazol-4-yl}-N-isopropylcyclopropane-carboxamide;
1-{5-[(2-amino-2-oxoethyl)amino]-3-cyano-1-[2,6-dichloro-4-pentafluorothiophenyl]-1H-pyrazol-4-yl}-cyclopropanecarboxamide;
methyl {4-[1-(aminocarbonyl)cyclopropyl]-3-cyano-1-[2,6-dichloro-4-pentafluorothiophenyl]-1H-pyrazol-5-yl}carbamate;
5-amino-1-[2,6-dichloro-4-pentafluorothiophenyl]-4-[1-(methylsulfonyl)cyclopropyl]-1H-pyrazole-3-carbonitrile;
1-{3-cyano-5-[(cyclopropylmethyl)(methyl)amino]-1-[2,6-dichloro-4-pentafluorothiophenyl]-1H-pyrazol-4-yl}cyclopropanecarboxamide;
ethyl {4-[1-(aminocarbonyl)cyclopropyl]-3-cyano-1-[2,6-dichloro-4-pentafluorothiophenyl]-1H-pyrazol-5-yl}carbamate;
or a pharmaceutically acceptable salt or prodrug thereof.

Still more preferred individual compounds of formula (I) are selected from:
1-{3-cyano-5-[(cyclopropylmethyl)amino]-1-[2,6-dichloro-4-pentafluorothiophenyl]-1H-pyrazol-4-yl}cyclopropanecarboxamide;
cyclopropylmethyl {4-[1-(aminocarbonyl)cyclopropyl]-3-cyano-1-[2,6-dichloro-4-pentafluorothiophenyl]-1H-pyrazol-5-yl}carbamate;
1-{5-amino-3-cyano-1-[2,6-dichloro-4-(trifluoromethoxy)phenyl]-1H-pyrazol-4-yl}-2,2-difluorocyclopropane-carboxamide;
1-{3-cyano-1-[2,6-dichloro-4-pentafluorothiophenyl]-5-[(2-fluoroethyl)amino]-1H-pyrazol-4-yl}cyclopropane-carboxamide;
1-{3-cyano-1-[2,6-dichloro-4-pentafluorothiophenyl]-5-(methylamino)-1H-pyrazol-4-yl}-2,2-difluoro-cyclopropanecarboxamide;
1-{5-chloro-3-cyano-1-[2,6-dichloro-4-pentafluorothiophenyl]-1H-pyrazol-4-yl}cyclopropanecarboxamide;
or a pharmaceutically acceptable salt or prodrug thereof.

Particularly preferred individual compounds of formula (I) are selected from:
cyclopropylmethyl {4-[1-(aminocarbonyl)cyclopropyl]-3-cyano-1-[2,6-dichloro-4-pentafluorothiophenyl]-1H-pyrazol-5-yl}carbamate;
1-{5-amino-3-cyano-1-[2,6-dichloro-4-pentafluorothiophenyl]-1H-pyrazol-4-yl}-cyclopropanecarboxamide;
1-{3-cyano-1-[2,6-dichloro-4-pentafluorothiophenyl]-5-[(2-fluoroethyl)amino]-1H-pyrazol-4-yl}cyclopropanecarboxamide;
1-{5-amino-3-cyano-1-[2,6-dichloro-4-pentafluorothiophenyl]-1H-pyrazol-4-yl}-2,2-dichlorocyclopropanecarboxamide;
isopropyl {4-[1-(aminocarbonyl)cyclopropyl]-3-cyano-1-[2,6-dichloro-4-pentafluorothiophenyl]-1H-pyrazol-5-yl}carbamate;
1-{3-cyano-1-[2,6-dichloro-4-pentafluorothiophenyl]-5-(methylamino)-1H-pyrazol-4-yl}-2,2-difluorocyclopropanecarboxamide;
1-{5-amino-3-cyano-1-[2,6-dichloro-4-pentafluorothiophenyl]-1H-pyrazol-4-yl}-2,2-difluorocyclopropanecarboxamide;
1-[3-cyano-1-[2,6-dichloro-4-pentafluorothiophenyl]-5-(methylamino)-1H-pyrazol-4-yl]cyclopropanecarboxamide; and
ethyl {4-[1-(aminocarbonyl)cyclopropyl]-3-cyano-1-[2,6-dichloro-4-pentafluorothiophenyl]-1H-pyrazol-5-yl}carbamate.

The most preferred compound of formula (I) is cyclopropylmethyl {4-[1-(aminocarbonyl)cyclopropyl]-3-cyano-1-[2,6-dichloro-4-pentafluorothiophenyl]-1H-pyrazol-5-yl}carbamate.

In the compounds according to formula (I) the term ‘halo’ means a group selected from fluoro, chloro, bromo or iodo. Preferably the term “halo” means a group selected from fluoro, chloro or bromo.

Alkyl, alkenyl, alkynyl and alkoxy groups, containing the requisite number of carbon atoms, can be unbranched or branched. The term lower alkyl shall be taken to mean C₁₋₆ alkyl. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy. Examples of alkenyl include methylene, 1,1-ethylene, 1,2-ethylene, 1,1-propylene, 1,2-propylene, 1,3-propylene and 2,2-propylene. The term cycloalkyl shall be taken to mean C₃₋₈ cycloalkyl. Examples of include cyclopropyl, cyclobutyl, cyclopentyl, cyclohexyl and cycloheptyl.

In the compounds according to formula (I) the term phenyl shall be taken to mean a six membered aromatic carbon ring, which phenyl can be substituted as described for compounds of formula (I).

In the compounds according to formula (I) the term “het” shall be taken to mean those substituents which fall into the definition as set out in claim 1. Preferably the term “het” shall be taken to mean those substituents which represent a five to six membered heterocyclic group, which is aromatic or non-aromatic, unsaturated, partially saturated or saturated and which contains one or more heteroatoms selected from nitrogen, N-oxide, oxygen, and sulphur and wherein said heterocyclic ring is optionally substituted where the valence allows with one or more substituents selected from halo, C₁₋₆ alkyl, C₁₋₆ haloalkyl, NR^{g}R^{h}, where R^{g} and R^{h} are independently selected from hydrogen, and C₁₋₆ alkyl. More preferably the term “het” shall be taken to mean those substituents which represent a five to six membered heterocyclic ring, which is aromatic or non-aromatic, unsaturated, partially saturated or saturated and which contains at least one nitrogen or oxygen atom and optionally up to two further heterocyclic atoms selected from nitrogen, oxygen and sulphur and wherein said heterocyclic ring is optionally substituted where the valence allows with one or more substituents selected from halo, C₁₋₆ alkyl, C₁₋₆ haloalkyl, NR^{g}R^{h}, where R^{g} and R^{h} are independently selected from hydrogen, and C₁₋₆ alkyl.

In the case of substituents R², R^{a}, or R^{b} and further optional substituents thereof of compounds of formula (I), the term “het” shall most preferably be taken to mean those substituents which represent a five to six membered heterocyclic ring, which is aromatic, unsaturated, or partially saturated and which contains at least one nitrogen atom and optionally up to two further heterocyclic atoms selected from nitrogen, oxygen and sulphur and wherein said heterocyclic ring is optionally substituted where the valence allows with one or more substituents selected from halo, and C₁₋₆ alkyl. Suitable preferred examples of such rings include 1-oxa-3,4-diazolyl, thiazolyl, 5-methyl-1-3,4-oxadiazol-2-yl, pyridinyl, or 1,2,4 triazolyl.

In the case of substituents R⁹, R^{e}, or R^{f} and further optional substituents thereof of compounds of formula (I), the term “het” shall most preferably be taken to mean those substituents which represent a five to six membered heterocyclic ring, which is aromatic, unsaturated, partially saturated, or saturated and which contains at least one nitrogen atom or one oxygen atom and optionally up to two further heterocyclic atoms selected from nitrogen, oxygen or sulphur and wherein said heterocyclic ring is optionally substituted where the valence allows with one or more substituents selected from halo, and C₁₋₆ alkyl. Suitable preferred examples of such rings include pyrazinyl, imidazolyl, pyridinyl, 1-hydroxy-pyridinyl, 1,2,4-triazolyl, 1,3,4-triazolyl, isoxaolyl, thiazolyl, 2-chloro-1,3-thiazol-4-yl, pyrazolyl, 1-methyl-1H-pyrazol-4-yl, 1-methyl-3-methyl-5-chloro-1H-pyrazol-4-yl, and tretrahydropyranyl.

In the compounds according to formula (I) each phenyl group may be optionally and independently substituted as set out in claim 1. More preferably each phenyl group may be optionally and independently substitueted with one or more further substitutents selected from the group consisting of halo, C₁₋₆ alkyl, C₁₋₆ haloalkyl, C₁₋₆ alkoxy, C₁₋₆ haloalkoxy, —NHS(O)ₙR¹¹, and S(O)ₙR¹¹. More preferably each phenyl group may be optionally substituted in the 4-position with a substituent selected from the group consisting of halo, C₁₋₆ haloalkyl, —NHS(O)ₙR¹¹, and S(O)ₙR¹¹.

In the case of substituents R⁹, R^{e}, or R^{f} and further optional substituents thereof of compounds of formula (I) it is preferred that each phenyl group may be optionally substituted in the 4-position a substituent selected from the group consisting of halo, C₁₋₆ haloalkyl, —NHS(O)ₙR¹¹, and S(O)ₙR¹¹. Suitable examples of such phenyl groups include 4-fluorophenyl, 4-trifluoromethylphenyl, (4-methylsulphonyl)phenyl, 4-[(methylsulphonyl)amino]phenyl, and 4-[(methylamino)sulphonyl]phenyl.

It will be understood that compounds of formula (I) may exist as one or more geometric isomers. Thus included within the scope of the present invention are all such possible geometric isomer forms of the compounds of the present invention. Geometric isomers may be separated by conventional techniques well known to those skilled in the art, for example, chromatography and fractional crystallisation.

It will be understood that compounds of formula (I) may exist as one or more tautomeric isomers. Thus included within the scope of the present invention are all such possible tautomeric isomer forms of compounds of the present invention.

It is to be understood that compounds of formula (I) may contain one or more asymmetric carbon atoms, thus compounds of the invention can exist as two or more stereoisomers. Included within the scope of the present invention are all stereoisomers such as enantiomers and diasteromers. Also included are acid addition or base salts wherein the counterion is optically active, for example, D-lactate or L-lysine, or racemic, for example, DL-tartrate or DL-arginine.

Pharmaceutically acceptable salts of the compounds of formula (I) include the acid addition and base salts thereof. Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

The following processes are illustrative of the general synthetic procedures which may be adopted in order to obtain the compounds of formula (I). It will be apparent to those skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

When one or more of R¹, R², R³, R⁴, R⁵, R⁶, R⁷, R⁸, R⁹, R¹⁰ and R¹¹ contain reactive functional groups then additional protection may be provided according to standard procedures during the synthesis of compounds of formula (I). In the processes described below, for all synthetic precursors used in the synthesis of compounds of formula (I), the definitions of R¹, R², R³, R⁴, R⁵, R⁶, R⁷, R⁸, R⁹, R¹⁰ and R¹¹, wherein R¹, R², R³, R⁴, R⁵, R⁶, R⁷, R⁸, R⁹, R¹⁰ and R¹¹ are as defined for formula (I), are intended to optionally include suitably protected variants, P¹, P², P³, P⁴, P⁵, P⁶, P⁷, P⁸, P⁹, P¹⁰ and P¹¹. Such suitable protecting groups for these functionalities are described in the references listed below and the use of these protecting groups where needed is specifically intended to fall within the scope of the processes described in the present invention for producing compounds of formula (I) and its precursors. When suitable protecting groups are used, then these will need to be removed to yield compounds of formula (I). Deprotection can be effected according to standard procedures including those described in the references listed below. For example, when R⁹ in formula (I) is an unsubstituted amino group, certain precursors may require protection of the amino group in order to perform the necessary transformations, for example, by an imidoformamide group such as a compound of formula (I), where R¹—R⁸ and R¹⁰ are as described for formula (I) and R⁹ represents —N═C(H)—NR^{c}R^{d}, where R^{c} and R^{d} independently represent C₁₋₆alkyl, e.g. to form a N,N-dimethyl group. Such imidoformamides may be prepared by standard methods, typically by refluxing the unprotected amine in N,N-dimethylformamide dimethyl acetal for 2-16 hours, usually around 5 hours followed by stirring at room temperature for 5-24 hours, usually overnight. The imidoformamide protecting group may be removed under standard conditions, such as at elevated temperature, with a suitable acid such as hydrochloric acid or para-toluenesulfonic acid in a solvent such as methanol or dioxane.

A compound of formula (I) may be prepared by cyclopropanation of an alkene of formula (II): 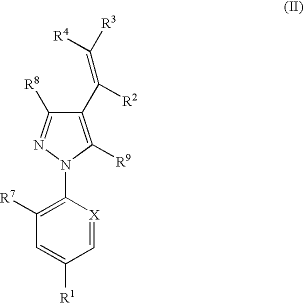
wherein R¹, R², R³, R⁴, R⁷, R⁸, R⁹ and X are as previously defined for formula (I). This may be achieved by in situ generation of the required carbenoid species, CR⁵R⁶ in which R⁵ and R⁶ are as previously defined for formula (I), in the presence of (II), by an appropriate method.

Such methods may include treatment of a compound of formula (II), with a reactive species such as trimethylsilyl difluoro(fluorosulfonyl)acetate (TFDA) at reflux in the presence of sodium fluoride, as described by Dolbier et al., in J. Fluor Chem., 2004, 125, 459, to yield a product of formula (I). Other methods for in situ carbenoid generation include treatment of chloroform or bromoform with base, preferably under phase transfer catalysis conditions, thermolysis of a suitable organometallic precursor such as an aryl trifluoromethyl, trichloromethyl, tribromomethyl or phenyl(trifluoromethyl) mercury derivative or treatment with a diazoalkane in the presence of a transition metal catalyst and treatment with a diazoalkane in the absence of a transition metal catalyst followed by thermolysis of the intermediate pyrazoline, or generation from a sulphur ylid.

Compounds of formula (II) can be synthesized using an organozinc reagent of formula (III): 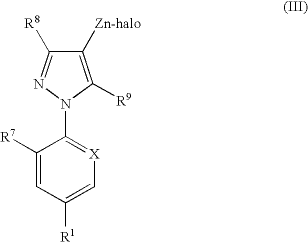
wherein R¹, R⁷, R⁸, R⁹ and X are as previously defined for formula (I). The organozinc reagent formula (III) may be obtained by treatment of (IV) wherein halo is preferably bromo or iodo, with activated zinc (Rieke zinc) in an aprotic solvent such as tetrahydrofuran, for several hours. The organozincate can then be cross coupled to a haloalkene in the presence of a palladium (II) species such as dichlorobis(triphenylphosphine) palladium (II) and a reducing agent such as diisobutylaluminium hydride in an aprotic solvent such as tetrahydrofuran, at elevated temperatures, normally at reflux.

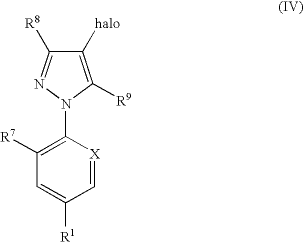

Alternatively, a compound of formula (II) may be obtained directly by the reaction of a compound of formula (IV) with an organostannane in the presence of a metal catalyst such as tetrakis(triphenylphosphine)palladium(0) at an elevated temperature for several hours.

Compounds of formula (IV) may be useful for accessing intermediates of formula (V).

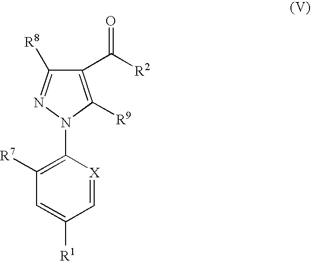

Thus, compounds of formula (IV) can be treated with a Grignard reagent such as isopropyl-magnesium chloride under inert conditions using an aprotic solvent at reduced temperature before treatment with an acid chloride or acid anhydride, upon warming to room temperature the desired ketone represented by formula (V) is produced.

Compounds of formula (V) can be utilized to access compounds of formula (II) wherein R³ and R⁴ are H. Thus compounds of formula (V) can be methylenated by treatment with a Wittig reagent under inert conditions at reduced temperature in a solvent such as tetrahydrofuran.

Compounds of formula (II) can also be obtained from compounds of formula (V), by treatment with a haloalkene such as dibromodifluoromethane in the presence of triphenylphosphine and Reike zinc in an aprotic solvent.

Similarly, a compound of formula (II) may be obtained by the reaction of a compound of formula (IV) with an organozinc reagent. A specific example is the compound of formula (VI), prepared as shown in Scheme 1 below. The reaction uses a metal catalyst such as tetrakis(triphenylphosphine)palladium(0) in a suitable solvent such as N,N-dimethylformamide at an elevated temperature, typically 110° C., for several hours, typically 10. Intermediates used in the synthesis of compound (VI) can be obtained using conventional synthetic procedures, in accordance with standard textbooks on organic chemistry or literature precedent.

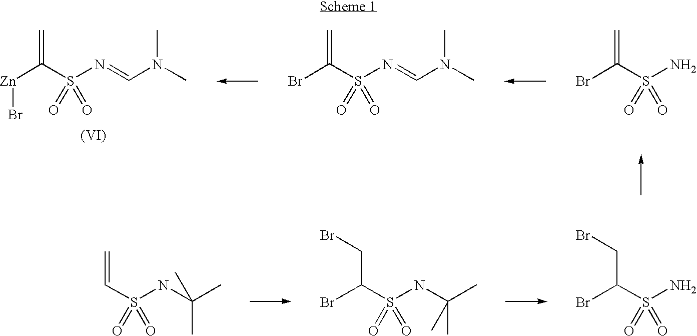

Alternatively, a compound of formula (VII), wherein R¹, R⁷, R⁸, R⁹ and X are as previously defined for formula (I) may be obtained by the reaction of a compound of formula (IV) with a suitable Grignard reagent such as isopropylmagnesium chloride followed by the addition of methyl pyruvate in a suitable solvent such as tetrahydrofuran.

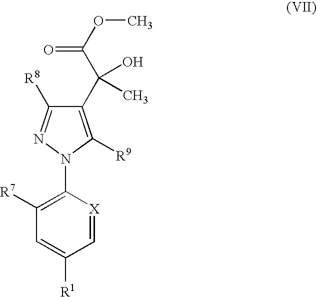

Subsequent dehydration using a mild base and an activating agent such as methanesulphonyl chloride gives a compound of formula (II) wherein R² is COOCH₃. Alternatively, dehydration can be achieved using a two step sequence of halogenation using thionyl chloride in acetonitrile followed by dehydrohalogenation by heating in an inert solvent such as para-xylene or by standard base catalysed dehydrohalogenation procedures.

A compound of formula (IV) may be obtained from a compound of formula (VII): 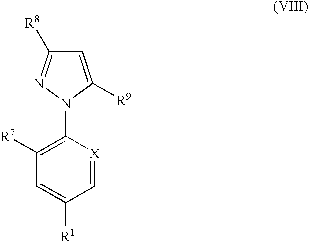
wherein R¹, R⁷, R⁸, R⁹ and X are as previously defined for formula (I), by conventional bromination or iodination procedures. For example, when halo is iodo, (VIII) is treated with N-iodosuccinimide in a suitable solvent such as acetonitrile at from about room temperature to about 85° C.

Alternatively, a compound of formula (IV) may be prepared as shown in Scheme 2 below: 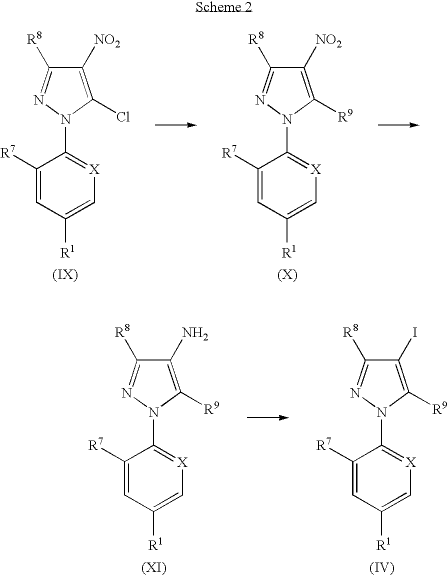
wherein R¹, R⁷, R⁸ and X are as previously defined for formula (I) and R⁹ is SR^{r}, NR^{r}R^{s} or OR^{r} wherein R^{r} and R^{s} are each independently H, alkyl, cycloalkyl, aryl, heteroaryl, cycloalkylalkyl, arylalkyl, heteroarylalkyl wherein each alkyl, cycloalkyl, aryl, heteroaryl, cycloalkylalkyl, arylalkyl, heteroarylalkyl may be optionally substituted. Compounds of formula (X) can be prepared from compounds of formula (IX) via standard nucleophilic substitution procedures. The amine (XI) may then be obtained by reduction using a suitable reducing agent, optionally in the presence of a catalyst, typically SnCl₂/HCl or Fe/CaCl₂. Compounds of formula (IV) may be prepared from (XI) by conventional Sandmeyer procedures.

A specific method for preparing a compound of formula (I), wherein R² is CF₂O, R³, R⁴ are F and R⁵, R⁶ are H is via an intermediate oxonium ion (XIII) formed by the reaction of a ketone of formula (XII) with TFDA in the presence of sodium fluoride, followed by hydride transfer and carbene insertion at the newly formed olefin to give the cyclopropane.

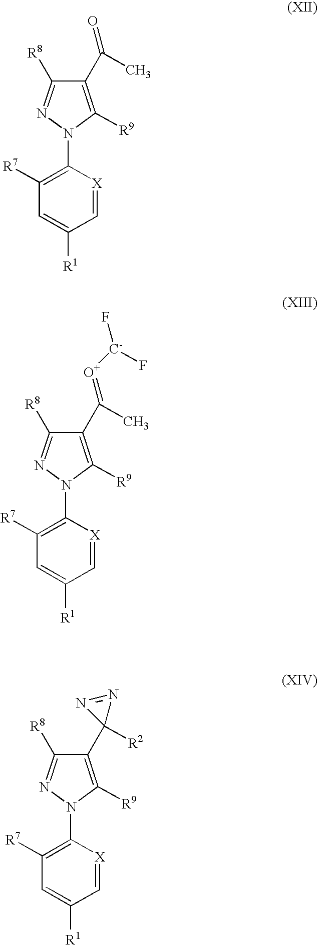

Another cyclopropanation procedure is via the reaction of a carbenoid species, generated in situ from compounds of formula (XIV), with alkenes of formula: 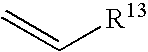
where R¹³ is optionally substituted aryl or heteroaryl. For example, a compound of formula (I) in which R² is CF₃ and R³ is 4-chlorophenyl may be obtained by stirring a compound of formula (XIV), wherein R² is CF₃ with 4-chlorostyrene in a suitable solvent, typically toluene, at 60° C. for an extended period of time, typically 18 hours.

The diazirine (XIV) may be prepared from the corresponding diaziridine using standard oxidising agents, such as iodine or those described in “Handbook of Reagents for Organic Synthesis—Oxidising and Reducing Agents” edited by S. D. Burke and R. L. Danheiser.

The diaziridine may be prepared by reacting compounds of formula (XV), wherein R¹, R², R⁷, R⁸, R⁹ and X are as defined for formula (I) 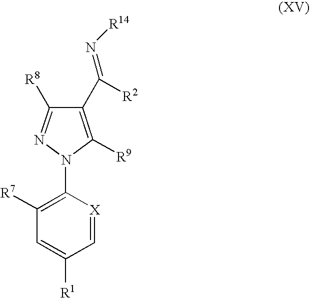
and R¹⁴ is tosyloxy, with ammonia gas at elevated pressure, followed by reaction with a suitable base such as triethylamine.

Furthermore, a compound of formula (I) may be prepared by the ring contraction of a 4,5-dihydropyrazole of formula (XVI), wherein R¹, R², R⁷, R⁸, R⁹ and X are as defined for formula (I) by heating at elevated temperatures in a suitable aprotic solvent such as xylene. An alternative extrusion method uses u.v. light in a suitable solvent, such as dichloromethane, in the presence of an initiator, such as benzophenone. This is particularly appropriate where R² is SO₂alkyl. During the preparation of compounds of formula (I) wherein R² is SO₂NH₂, the sulphamoyl group may need protection as the sulphonimido-formamide.

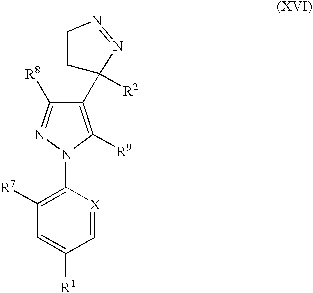

The dihydropyrazoles are prepared from compounds of formula (II), wherein R¹, R², R⁷, R⁸, R⁹ and X are as defined for formula (I), by standard literature procedures.

Arylpyrazoles of formula (I) may also be prepared by the Japp-Klingemann reaction. This reaction is described in Org. React., 1959, 10, 143-178. 3,4,5-Trisubstituted 1-arylpyrazoles may be produced directly in a reaction which involves coupling of an aryldiazonium species with an appropriately substituted precursor bearing a desired substituent. The desired substituent is introduced concomitantly at the C-4 position in a process, which does not involve any rearrangement. Furthermore, a very wide variety of 4-substituents may be introduced conveniently and directly.

Thus, a compound of formula (I) in which R⁹ is NH₂, can be prepared by reacting a compound of formula (XVII) 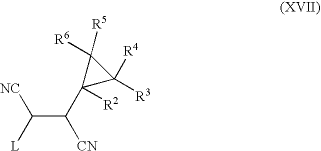
with a compound of formula (XVIII) 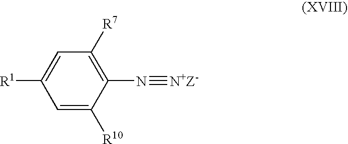
optionally in the presence of an acid, wherein:
R¹ to R¹⁰ are as defined above in relation to the compounds of formula (I);
L is an activating group; and
Z is a compatible counter ion, followed by removal of group L.

The counter ion Z⁻ may be any suitable counter ion normally found in diazonium reactions. Preferably, Z⁻ is halogen, HSO₄⁻, or tetrafluoroborate and most preferably is tetrafluoroborate.

The group L is an electron withdrawing group which stabilises the anion intermediate in the process. Thus, preferably, L is a group which is capable of stabilising a negative charge on an adjacent carbon atom. The group L must also be removable. L can be removed under basic conditions, for example by base hydrolysis or can be removed by reduction and/or elimination. The group L is important as it serves to direct the reaction of the diazonium species with the compound of formula (XVII) but then is removed in the subsequent stages of the reaction. Preferably L is an ester group or a group COR¹⁵. More preferably, L is a group selected from: —S(O)ₚR¹⁶ where p is 1 or 2, (R¹⁶O)₂PO, COOR¹⁶ and —COR¹⁵, wherein R¹⁵ is selected from: C₁₋₈ alkyl, di-C₁₋₈ alkylamino, C₁₋₈ alkylthio, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₃₋₈ cycloalkyl and C₃₋₈ halocycloalkyl; and R¹⁵ can be hydrogen; and wherein R¹⁶ is selected from: C₁₋₈ alkyl, C₃₋₈ cycloalkyl, (CH₂)ₙPh and (CH₂)ₙ heteroaryl wherein n=0, 1 or 2, each of which groups may be optionally substituted on any carbon atom by one or more groups selected independently from: halogen, hydroxy, cyano, nitro, C₁₋₄ alkoxy, C₁₋₄ haloalkoxy, C₁₋₄ alkanoyl, C₁₋₄ haloalkanoyl, C₁₋₄ alkylsulphinyl, C₁₋₄ haloalkylsulphinyl, C₁₋₄ alkylsulphonyl, C₁₋₄ haloalkylsulphonyl, C₃₋₈ cycloalkyl and C₃₋₈ halocycloalkyl; and R¹⁵ can be hydrogen. Preferably L is a group selected from COR¹⁵ and COOR¹⁶. Most preferably L is —COOMe or —COOEt.

In certain cases, the nature of the leaving group L means that the resulting intermediate is in the wrong oxidation state. Thus, where necessary, one or more reaction steps may be added to ensure the correct oxidation state is reached prior to cyclising to form the aryl pyrazole.

Ideally, for the coupling reaction to form the compound of formula (I), the solvent should be a polar solvent which does not react with either the diazonium salt or cation, or with the compound of formula (XVII). The reaction may optionally be carried out under mildly acidic conditions.

The diazonium salt of formula (XVIII) can be produced by conventional means and may be prepared in situ for further reaction or can be isolated and used in a subsequent reaction step. For example, by the dropwise addition of a solution of the corresponding aminobenzenes in glacial acetic acid to a solution of sodium nitrite in concentrated sulphuric/glacial acetic acid mixtures at reduced temperature, typically 10° C., followed by heating at 50° C. for several hours, typically 1 hour and allowing to cool to room temperature. This solution of the diazonium salt is then added dropwise to a solution of a compound of formula (XVII) in a suitable solvent, such as acetic acid followed by stirring at room temperature for up to 1 hour. The reaction mixture is poured into water and extracted with a water immiscible organic solvent such as dichloromethane. Aqueous ammonium hydroxide is added to the organic extract and stirred overnight to give compounds of formula (I). The aminobenzenes are generally commercially available. Others may be prepared by standard literature procedures. For example (XX) is readily prepared from (XIX) by chlorination using N-chlorosuccinimide in acetonitrile.

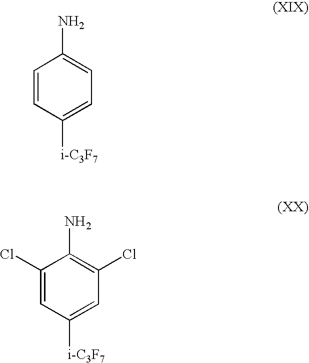

Alternatively, compounds of formula (XVII) can be obtained from compounds of formula (XXI) wherein R², R³, R⁴, R⁵, R⁶ and L are as defined for formula (XVII), for example, by treating a compound of formula (XXI) with a source of cyanide ions.

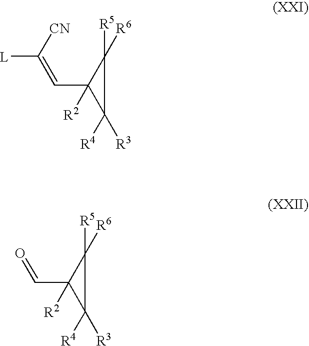

Compounds of the formula (XXI) can be obtained by reducing and then dehydrating a compound of formula (XXIII).

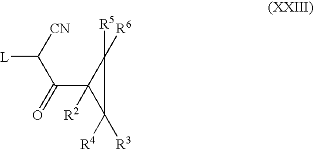

Compounds of formula (XXIII) can, for example, be made by condensation of an alkyl cyanoalkanoate e.g. methyl cyanoacetate with an acid chloride in an aprotic solvent such as dichloromethane in the presence of a Lewis acid, such as magnesium chloride and a mild base, such as triethylamine, at reduced temperature.

Alternatively, compounds of formula (XXI) can be accessed by Knoevenagel condensation of a suitable aldehyde, such as (XXII) or ketone with an alkyl alkanoate such as methyl cyanoacetate. Compounds of formula (XXII) in which R²=COOalkyl can be prepared by selective reduction of the malonyl esters (XXIV)

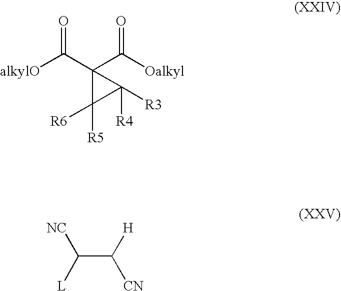

Compounds of formula (XXV) wherein L=CO₂C₁ to C₆ alkyl are synthesised by the slow addition of glycolonitrile optionally at decreased temperatures to a C₁ to C₆ alkyl cyanoacetate, in an aprotic solvent such as dimethylformamide, followed by the addition of a base such as potassium carbonate.

In addition, variations to the Japp-Klingemann reaction, utilising standard conditions well-known to those skilled in the art, for producing compounds of formula (I) and its precursors, are also intended to fall within the scope described in the present invention. For example, coupling of an aryldiazonium species with precursors of formula (XXVI): 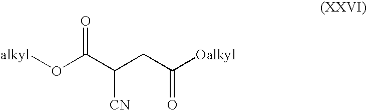
in the presence of a suitable base, may be useful in accessing compounds in which R⁹ is OH. These compounds may then undergo standard alkylation, acylation, carbamoylation, sulphonation and other procedures to produce, for example, the corresponding alkoxy derivatives.

Alternatively, arylpyrazoles may be prepared by the reaction of optionally substituted phenylhydrazine derivatives with compounds of formula (XXVII) or (XXVIII): 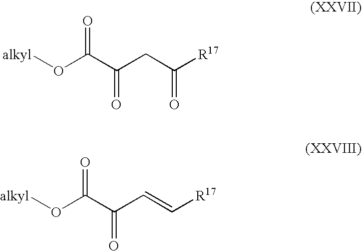
in which R¹⁷ is lower alkyl or cycloalkyl.

In another aspect, the invention provides processes for the preparation of compounds of formula (I) from alternative compounds of formula (I) through functional group interconversion. For example, saponification of a compound of (I) in which R² is a methyl ester to give the acid, may be achieved using standard ester hydrolysis conditions. A particularly useful procedure involves adding tetrahydrofuran, water and lithium hydroxide and stirring at room temperature for from 1 to 60 h or by the addition of pyridine and lithium iodide and heating at elevated temperatures for an extended period of time. This acid can be further reacted with secondary, tertiary or cyclic amine compounds or ammonia or ammonium hydroxide in the presence of a suitable base such as triethylamine and an activating agent, such as ethyl chloroformate, in a suitable solvent such as tetrahydrofuran to give the amide derivative. For example, to a compound of formula (I) in which R² is CO₂H in tetrahydrofuran and triethylamine, cooled to 0° C. can be added ethyl chloroformate, cyclopropylmethylamine and in tetrahydrofuran and allowed to warm to room temperature to give a compound of formula (I) in which R² is cyclopropanecarboxamide.

Compounds of formula (I), in which R² is a carboxylic acid, can be reduced by standard literature procedures, such as sodium borohydride, to give the corresponding alcohol.

Furthermore, compounds of formula (I), in which R² is a carboxylic acid, can rearrange under standard Curtius conditions to carbamates which after deprotection gave compounds of formula (I) wherein R² is NH₂.

Using standard reaction conditions, compounds of formula (I), wherein R² is an alkyl ester may be converted to amides, wherein R² is CONH₂. For example, trimethyl aluminium in hexane is added to ammonium chloride in a suitable solvent, typically toluene, at 0° C., optionally under nitrogen. After stirring for 1-2 h at room temperature, a solution of a compound of formula (I), wherein R² is COOalkyl, in a suitable solvent is added. Conversion to the amide is achieved by stirring at elevated temperature, typically 50° C. for 15-80 hours. Similarly, transesterifications may be achieved by reaction with a substituted alcohol and hydroxylamides (R² is CONHOH) prepared by reaction with hydroxylamine. Acylhydrazones and bis-acylhydrazones may be similarly prepared using literature conditions. These bis-acylhydrazones may be converted to 1,2,4-oxadiazoles by reaction with phosphorus oxychloride in a suitable solvent. The acylhydrazones may be converted to 1,2,4-oxadiazoles by refluxng with triethyl orthoformate in the presence of an acid catalyst, typically p-toluenesulphonic acid. These 1,2,4-oxadiazoles can be hydrolysed back to the acylhydrazones by refluxing in a suitable solvent, such as methanol:dioxane mixtures, in the presence of an acid, such as hydrochloric acid.

Compounds of formula (I) in which R² is an amide may undergo standard alkylation reactions with compounds of formula R¹—Y, in which Y is a suitable leaving group, to give the substituted amide. Compounds of formula (I) in which R² is an amide may undergo a functional group interconversion by refluxing with Lawesson's reagent for several hours in a suitable solvent, typically tetrahydrofuran, to produce the thioamide or be dehydrated by reaction with trifluoroacetic anhydride and 1,4-dioxane in pyridine at 0° C. for several hours to give the nitrile, wherein R² is CN.

In particular, a compound of formula (XXIX), wherein R¹—R⁸ and X are as defined for formula (I), can be cyclised to (XXX) via the acid catalysed addition of an aldehyde to give the imine intermediate followed by the in situ reduction using a suitable reducing agent, such as sodium borohydride.

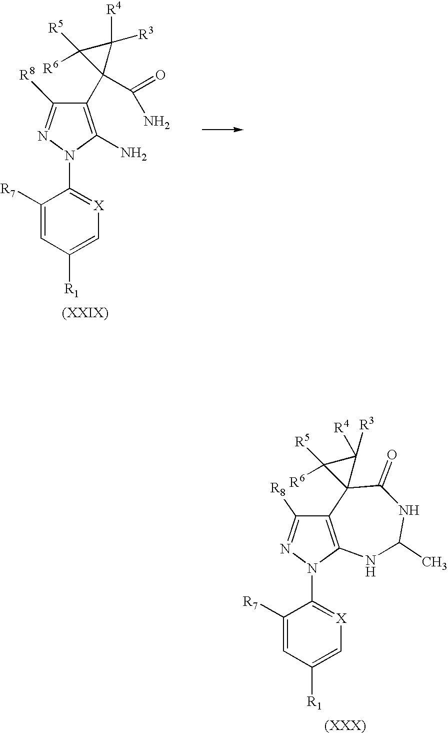

Compounds of formula (I) in which R² is aminomethyl may be obtained via formation of the thioalkylated intermediate formed by treatment of (I) in which R² is a thioamide, with an alkylating agent such as triethyloxonium tetrafluoroborate, in a suitable solvent, typically dichloromethane, at 0° C. and then by being allowed to stir at room temperature for an extended period of time, followed by reduction with sodium borohydride at 0° C.

Compounds of formula (I) in which R² is thioamide may be reacted with haloketones or haloaldehydes to give (I) in which R² is substituted thiazole. Similarly, reaction with acylhydrazides to give compounds of formula (I) in which R² is substituted triazole.

Compounds of formula (I) in which R² is aminomethyl can be further treated with an acid anhydride, in a suitable solvent, typically dichloromethane and a mild base such as triethylamine and stirring at room temperature for an extended period of time, typically 60 h, to give the corresponding amide.

Furthermore compounds of formula (I) in which R² is aminomethyl can be monosulphonated or disulphonated with alkyl or aryl sulphonyl halides under standard conditions well-known to those skilled in the art.

Compounds of formula (I) in which R² is halo can undergo standard nucleophilic substitution reactions by refluxing with a suitable acid catalyst such as p-toluenesulphonic acid and an alkylthiol or alcohol for an extended period of time, typically from 18 hours to several days, to produce the corresponding ether or thioether respectively. Compounds of formula (I) in which R² is S-alkyl can be oxidised to the corresponding sulphines or sulphones using standard oxidizing agents, such as m-chloroperoxybenzoic acid or those described in “Handbook of Reagents for Organic Synthesis—Oxidising and Reducing Agents” edited by S. D. Burke and R. L. Danheiser

Compounds of formula (I) in which R² is formyl can undergo standard literature procedures for transformation of aldehydes. For example, reaction with (trifluoromethyl)trimethylsilane in a suitable solvent, such as tetrahydrofuran, in the presence of tetrabutylammonium fluoride gives intermediates of formula (XXXI). These intermediates can be desilylated using tetrabutylammonium fluoride in tetrahydrofuran to give secondary alcohols of formula (XXXII)

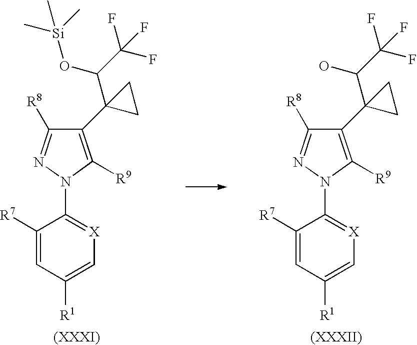

Compounds of formula (I) in which R² contains a secondary alcohol can be oxidized, for example by stirring with Dess Martin Periodinane at room temperature for 30 minutes in a suitable solvent, typically dichloromethane, to produce the corresponding ketone. Compounds of formula (I) in which R² contains a primary alcohol can be oxidized, for example by stirring with Dess Martin Periodinane at room temperature for 30 minutes in a suitable solvent, typically dichloromethane, to produce the corresponding aldehyde, for example, R²=hydroxymethyl can be readily converted to R²=formyl. Compounds of formula (I), in which R²=hydroxymethyl can be prepared by reduction of the acids of formula (I), wherein R²=—COOH. The acid can be activated by reaction with ethylchloroformate in the presence of a base, such as triethylamine in a suitable solvent, such as tetrahydrofuran; subsequent reduction can be effected using, for example, sodium borohydride.

Compounds of formula (I) in which R⁹ is NH₂ may be used to synthesis imines by reacting the amino functionality of formula (I) with aldehydes and an appropriate acid catalyst, typically p-toluenesulphonic acid at room temperature, for an extended period of time, typically 16 h or with aldehydes in the presence of a mild reducing agent such as sodium triacetoxyborohydride and a mild base to form secondary amines. For example, a compound of formula (I) in which R⁹ is NH₂ undergoes reaction with isonicotinaldehyde and a mild base to give the corresponding imine functionality which can be further reduced by reaction with a suitable reducing agent such as sodium borohydride to give the secondary amine. This can be further oxidized using standard procedures to give the N-oxide. Similarly, compounds of formula (I) in which R⁹ is NH₂ may be reacted with optionally substituted ketones.

N-alkylation, N-arylalkylation and N-heteroarylalkylation of compounds of formula (I) in which R⁹ is NH₂ can also be effected by reaction with the appropriate organic halides using a strong base, such as sodium hydride in a suitable aprotic solvent, for example N-methylpyrrolidone. Reactions are stirred at room temperature for 10-25 hours, typically overnight. Those skilled in the art will recognize that using a suitable sequence of synthetic procedures both mono-N-substituted and di-N-substituted products may be obtained. More reactive alkyl halides need less severe reaction conditions. For example, compounds of formula (I) in which R⁹ is NH₂ will react with tert-butyl bromoacetate in a suitable solvent, such as acetonitrile in the presence of a weak base, typically potassium carbonate at elevated temperatures, typically 55° C.

Compounds of formula (I) in which R⁹ is NH₂ may be carbamoylated by stirring with phosgene in a suitable solvent, typically dichloromethane, in the presence of a base, such as pyridine, at 0° C., followed by reaction with a primary, secondary or tertiary alcohol at room temperature for 10-30 hours, typically overnight. Compounds of formula (I) in which R⁹ is NH₂ may also be carbamoylated by reacting with chloroformates using standard literature conditions.

Reductive amination of compounds in which R⁹=NH₂ can also be achieved with protected aldehydes, such as (XXXIII)

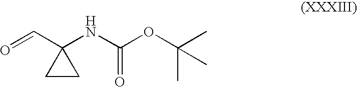

The t-BOC protecting group can be removed using standard procedures such as stirring with trifluoroacetic acid in a suitable solvent, such as dichloromethane for several hours, usually 2 hours, at room temperatures yielding compounds of formula (XXXIV)

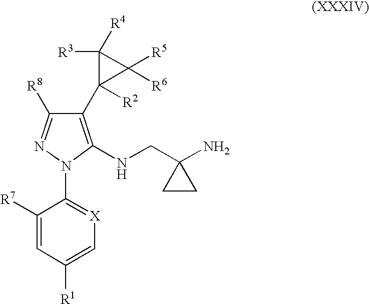

The primary amine in compounds of formula (XXXIV) can be alkylated, acylated and sulphonylated using classical literature procedures. Typical sulphonylation procedures are reaction with a sulphonyl chloride in a suitable solvent, such as dichloromethane, in the presence of a base, such as triethylamine.

Reductive amination of compounds in which R⁹=NH₂ can also be achieved with protected aldehydes, such as (XXXV). The t-BOC protecting group can be removed using trifluoroacetic acid in dichloromethane.

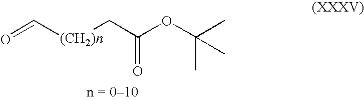

Compounds of formula (I) in which R⁹ is NH₂, can undergo reaction with triethyl orthoformate in acidic conditions, by heating at elevated temperatures, typically 60° C., for several hours, typically from 2 to 4 hours, to give (I) in which R⁹ is —N═CHOC₂H₅. This can, in turn, be further reduced by a suitable reducing agent, such as sodium borohydride, to give a compound of formula (I) in which R⁹ is —NHCH₃. Compounds of formula (I) in which R⁹ is NH₂ may be functionalised in a similar manner

A compound of formula (I) in which R⁹ is H, may be prepared by the diazotisation of a compound of formula (I) in which R⁹ is NH₂ by a variety of standard diazotisation procedures.

In a similar manner, compounds of formula (I) in which R⁹ is —S-alkyl, may be formed by coupling the diazonium species formed from a compound of formula (I) in which R⁹ is NH₂ and an appropriate nucleophile such as (alkylS)₂. Furthermore, compounds of formula (I) in which R⁹ is S-alkyl may be oxidised, using standard oxidising agents, such as hydrogen peroxide, to give the corresponding sulphines and sulphones.

Compounds of formula (I) in which R⁹ is NH₂, can be converted to give a compound of formula (I) wherein R⁹ is halo, utilising standard Sandmeyer reaction conditions. These halo compounds may be used in standard organometallic coupling procedures, for example in the preparation of a compound of formula (I) in which R⁹=—CF₃.

Compounds of formula (I) in which R⁹ is CH₂Y or N-alkyl-Y, in which Y is a suitable leaving group such as halo, may, in the presence of a suitable base, undergo a wide range of nucleophilic substitution reactions well known to those skilled in the art. Examples of such nucleophiles are cyanide ion, alcohols, phenols, thiols, primary and secondary amines and heterocycles such as 1,2,4-triazole. A typical leaving group is the mesyl group; such compounds are prepared from compounds in which Y═OH by reaction with methane sulphonyl chloride in acetonitrile in the presence of triethylamine.

Furthermore, compounds of formula (I) in which R⁹ is —NH₂ or aminoalkyl can be monosulphonated or disulphonated with alkyl or aryl sulphonyl halides under standard conditions well-known to those skilled in the art, to give the corresponding sulphonamides.

Furthermore, compounds of formula (I) in which R⁹ is —NH₂ or aminoalkyl can be acylated under standard conditions well known to those skilled in the art. The resulting amides can be reduced to amines by reaction with phosphorus pentachloride in toluene at reflux, cooling to room temperature and pouring into sodium borohydride in a polar hydroxylic solvent, such as methanol.

Compounds of formula (I) in which R⁹ is —NH₂, may also be converted to compounds of formula (I) in which R⁹ is —CH₃ or —CHF₂ as shown in Scheme 3 below. Firstly, compounds (XXXVI) may be converted to (XXXVII) by the radical arylation of methyl acrylate with the corresponding diazonium salts. Compounds of formula (XXXVII) can be dehydrobrominated using standard conditions by stirring with base, such as DBU, for several hours, to give enones, (XXXVIII). Conversion of (XXXVIII) to (XXXIX) can be achieved via diol formation, utilising OSO₄, followed by oxidative cleavage, using an oxidising agent such as sodium periodate, to generate the aldehyde. Aldehydes of formula (XXXIX) may be reduced to give alcohols of formula (XL) by stirring with a reducing agent, typically sodium borohydride or reacted further with a halogenating reagent such as diethylaminosulfur trifluoride to obtain a compound of formula (I) in which R⁹ is difluoromethyl. Reaction of (XL) with thionyl chloride and heating at reflux for several hours gives the intermediate chloro derivative from compounds of formula (XLI) may then be obtained by reduction, for example using Rieke zinc.

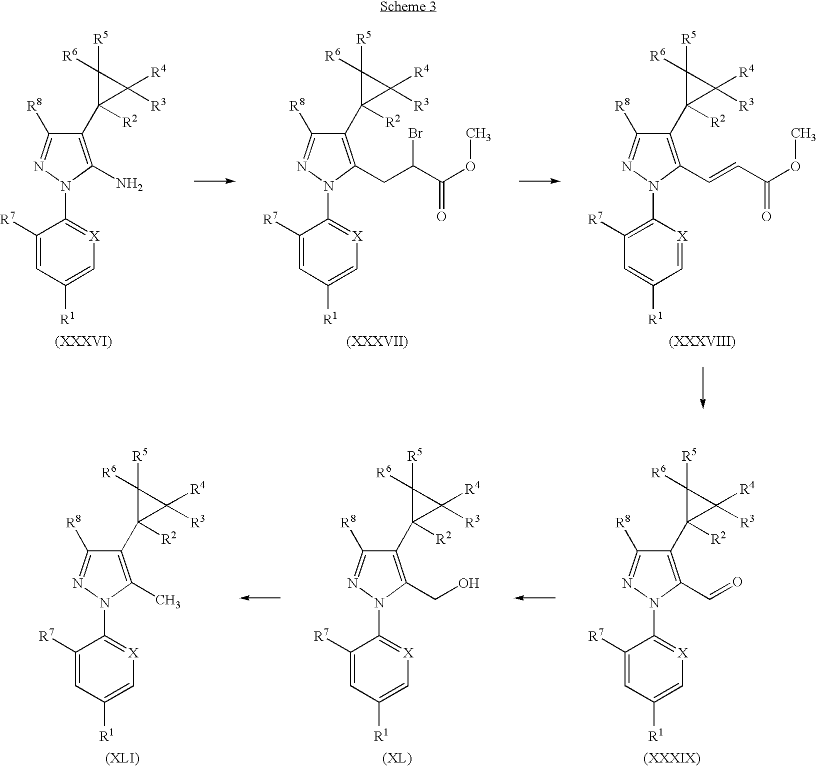

Compounds of formula (XXXIX) and (XL) may be used to prepare compounds of formula (I) in which R⁹ encompasses a wide variety of carbon linked substituents. Also, in (XL), activation of the hydroxyl, such as by mesylation or tosylation, gives an intermediate that undergoes a wide range of nucleophilic substitution reactions. Compounds of formula (XL) can also be acylated and alkylated using standard literature procedures. For example by reaction with an alkyl halide, such as iodomethane, in a suitable solvent, typically acetonitrile, in the presence of a base, such as potassium carbonate at room temperature for several days, typically 5 days. The aldehyde, (XXXIX) may be readily converted to the acid, nitrile, esters, amides and thioamides under standard conditions well-known to those skilled in the art. Standard Wittig olefination of the aldehyde (XXXIX) may be followed by routine cyclopropanation procedures to give compounds in which R⁹ is substituted cyclopropyl. For example, methylenation may be achieved using the Wittig reaction, using a Peterson reagent, using a Tebbe reagent or using the Lombardt procedure. A typical Wittig reaction involves adding n-butyllithium in hexane to a solution of methyltriphenylphosphonium bromide in tetrahydrofuran at 0° C. followed by addition of a solution of an aldehyde of formula (XXIX) in tetrahyd rofu ran giving compounds of formula (I) in which R⁹=vinyl. Organometallic addition to the aldehyde, (XXXIX), followed by oxidation of the secondary alcohol, then Wittig olefination and cyclopropanation may be used to prepare compounds of formula I), for example wherein R¹²=—CF₃.

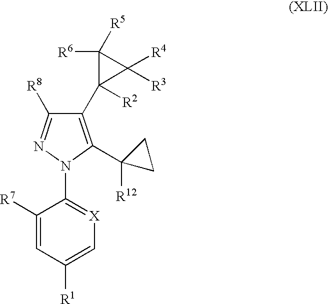

Alternatively, organometallic addition to the aldehyde, (XXXIX), followed by elimination of the hydroxyl group using standard procedures such as reaction with SOCl₂ in the presence of a zinc catalyst, may be a means to generate compounds of formula (I) in which R⁹ is optionally substituted alkyl, optionally substituted aryl or arylalkyl and optionally substituted heteroaryl or heteroarylalkyl. Compounds of formula (XXXIX) may also undergo standard Knovenagel type reactions, followed by reduction and partial hydrolysis and heating at elevated temperature to give the corresponding ester derivative which may be further derivatised. Alternatively, methylenation of compounds of formula (XXXIX) may be readily achieved utilising standard known reactions such as the Wittig or the Horner-Wadsworth-Emmons reaction. The resulting compounds of formula (I) in which R⁹ is vinyl, may be hydroxylated using standard conditions such as by reaction with hydrogen peroxide and a suitable base to give compounds in which R⁹ is —CH₂CH₂OH. These compounds can, in turn, be further oxidised to give the corresponding aldehydes and acids, i.e. where R⁹ is —CH₂CHO or —CH₂COOH. These aldehydes undergo reactions well known to those skilled in the art, such as Wittig olefination and reductive amination. The acids undergo the Curtius rearrangement to give compounds of formula (I), in which R⁹ is —CH₂NH₂, which may be alkylated, acylated, sulphonylated and other electrophiles. Furthermore, compounds in which R⁹ is —CH₂CH₂OH may be activated for example by the addition of SOCl₂ or TsCl and further reacted with a wide range of nucleophiles such as ⁻CN, ⁻SR or ⁻OR to achieve the corresponding alkylated derivative.

Alternatively, standard known catalysed cross coupling reactions, such as the Heck reaction, may be employed to generate compounds of formula (I) in which R⁹ is substituted vinyl from the vinyl derivative.

Oxidation of compounds of formula (XXXIX) using standard reaction conditions followed by further derivatisation of the acid formed may be a means of accessing compounds of formula (I) in which R⁹ is a heterocyclic moiety. For example, the oxidised product may undergo reaction with substituted acyl hydrazides to give oxadiazoles. Those skilled in the art will recognise that a wide variety of optionally substituted heterocycles may be synthesised from the aldehydes (XXXIX) or the corresponding acids. These acids may also be derivatised using standard literature procedures.

A compound of formula (I) in which R⁸ is —C(O)SCH₃ may be prepared from (I) R⁸=—CN by the acid catalysed addition of methanethiol by heating under pressure at elevated temperatures, typically 80° C. for several hours, typically 16. Compounds of formula (I) in which R⁸ is —CN may undergo reactions of nitriles as recorded in organic chemistry textbooks and literature precedent.

It will also be appreciated by persons skilled in the art that, within certain of the processes described, the order of the synthetic steps employed may be varied and will depend inter alia on factors such as the nature of other functional groups present in a particular substrate, the availability of key intermediates, and the protecting group strategy (if any) to be adopted. Clearly, such factors will also influence the choice of reagent for use in the said synthetic steps.

The skilled person will appreciate that the compounds of the invention could be made by methods other than those herein described, by adaptation of the methods herein described and/or adaptation of methods known in the art, for example the art described herein, or using standard textbooks such as “Comprehensive Organic Transformations—A Guide to Functional Group Transformations”, RC Larock, Wiley-VCH (1999 or later editions).

It is to be understood that the synthetic transformation methods mentioned herein are exemplary only and they may be carried out in various different sequences in order that the desired compounds can be efficiently assembled. The skilled chemist will exercise his judgement and skill as to the most efficient sequence of reactions for synthesis of a given target compound.

2. Second Component

The second active component may be selected from the macrocyclic lactone class of compounds (such as ivermectin, avermectin, abamectin, emamectin, eprinomectin, doramectin, selamectin, moxidectin, nemadectin, milbemycin and milbemycin derivatives), benzimidazoles (such as albendazole, cambendazole, fenbendazole, flubendazole, mebendazole, oxfendazole, oxibendazole and parbendazole), imidazothiazoles and tetrahydropyrimidines (such as tetramisole, levamisole, pyrantel pamoate, oxantel or morantel), derivatives and analogues of the paraherquamide/marcfortine class of anthelmintic agents, nitroscanate, antiparasitic oxazolines (such as those disclosed in U.S. Pat. No. 5,478,855, U.S. Pat. No. 4,639,771 and DE-19520936), derivatives and analogues of the general class of dioxomorpholine antiparasitic agents as described in WO-9615121, cyclic depsipeptides (such as those described in WO-9611945, WO-9319053, WO-9325543, EP-626375, EP-382173, WO-9419334, EP-382173, and EP-503538, and particularly emodepside), fipronil; pyrethroids; organophosphates; insect growth regulators (such as lufenuron); ecdysone agonists (such as tebufenozide and the like); spinosyns (such as Spinosad), amidoacetonitriles (such as those disclosed in WO-2005044784), and neonicotinoids (such as imidacloprid and the like). Optionally, a third active component chosen from this list may also be used.

In a preferred embodiment, the second component has anthelmintic activity.

In another preferred embodiment, the second component is a macrocyclic lactone selected from ivermectin, avermectin, abamectin, emamectin, eprinomectin, doramectin, selamectin, moxidectin, nemadectin, milbemycin and milbemycin derivatives.

In a more preferred embodiment, the second component is a milbemycin or milbemycin derivative.

Milbemycins are a family of macrolides originally isolated from Streptomyces hygroscopicus. For example, see A. Aoki et al., DE 2329486 and U.S. Pat. No. 3,950,360, both assigned to Sankyo. Milbemycins for use in the present invention may be obtained by a fermentation process or by total synthesis, or by synthetic modification of a fermentation product. Examples of milbemycins include milbemycin A₃, milbemycin A₄ and milbemycin D.

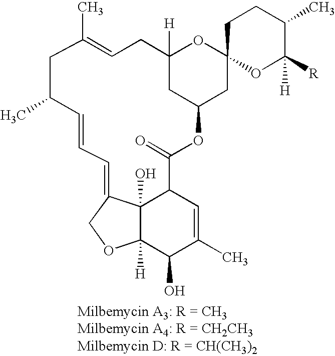

Preferred milbemycins include milbemycin A₃ and milbemycin A₄, and mixtures thereof. A particularly preferred mixture is milbemectin, which comprises milbemycin A₃ and milbemycin A₄ in a 3:7 ratio.

Milbemycin derivatives are compounds that can be prepared by synthetic modification of milbemycins. A preferred milbemycin derivative is milbemycin oxime, described in J. Ide et al., EP 110667 and U.S. Pat. No. 4,547,520, both assigned to Sankyo, which is a mixture of two components, milbemycin A₃ oxime and milbemycin A₄ oxime, in a ratio of approximately 2:8.

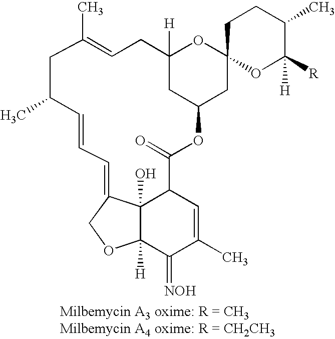

The two components may be administered simultaneously, sequentially or separately.

As used herein, simultaneous administration means the administration of both components to the host animal in a single action, which requires the two components to be incorporated into a single dosage unit, such as a single tablet or a single pour-on solution.

Sequential administration means the administration of each component is a separate action, but the two actions are linked. For example, administering a tablet comprising one component and a second tablet comprising the second component is considered to be sequential administration, even if the two tablets are given to the host animal at the same time.

Separate administration refers to the administration of each component independently of the other.

For convenience, simultaneous administration may be preferable.

The components may be administered by any suitable route. Examples of suitable routes of administration include oral, topical and parenteral administration. The choice of the route will depend on the species of the host animal and the nature of the parasitic infestation. For example, oral administration might be preferred in the case of a human or companion animal host, while topical administration might be more convenient for treating large numbers of livestock animals such as a herd of cattle. Where the two components are administered sequentially or separately then they may both be given by the same route, or they may be administered by different routes.

The components may be administered alone or in a formulation appropriate to the specific use envisaged. Generally, they will be administered as a formulation in association with one or more pharmaceutically acceptable excipients. The term “excipient” is used herein to describe any ingredient other than the active components. The choice of excipient will to a large extent depend on factors such as the particular mode of administration, the effect of the excipient on solubility and stability, and the nature of the dosage form.

The components may be administered as crystalline or amorphous products, for example, spray-dried dispersions or as produced by melt-extrusion or nano-milling. They may be obtained, for example, as solid plugs, powders, or films (for example, rapid dissolving or mucoadhesive films) by methods such as precipitation, crystallization, freeze drying, or spray drying, or evaporative drying. Microwave or radio frequency drying may be used for this purpose.

The methods by which the components may be administered include oral administration by capsule, bolus, tablet, powders, lozenges, chews, multi and nanoparticulates, gels, solid solution, films, sprays, or liquid formulation. Liquid forms include suspensions, solutions, syrups, drenches and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. Oral drenches are commonly prepared by dissolving or suspending the active ingredient in a suitable medium.

Thus compositions useful for oral administration may be prepared by mixing the active ingredient with a suitable finely divided diluent and/or disintegrating agent and/or binder, and/or lubricant etc. Other possible ingredients include anti-oxidants, colourants, flavouring agents, preservatives and taste-masking agents.

For oral dosage forms, depending on dose, the drug may make up from 1 wt % to 80 wt % of the dosage form, more typically from 5 wt % to 60 wt % of the dosage form. Examples of suitable disintegrants for use herein include sodium starch glycolate, sodium carboxymethyl cellulose, calcium carboxymethyl cellulose, croscarmellose sodium, crospovidone, polyvinylpyrrolidone, methyl cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Examples of suitable binders for use herein include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Examples of diluents include lactose (monohydrate, spray-dried monohydrate, anhydrous and the like), mannitol, xylitol, dextrose, sucrose, sorbitol, microcrystalline cellulose, starch and dibasic calcium phosphate dihydrate.

Oral formulations may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Lubricants include magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The components may be administered topically to the skin, that is dermally or transdermally. The compounds may also be administered via the mucosa or mucous membranes. Typical formulations for this purpose include pour-on, spot-on, dip, spray, mousse, shampoo, powder formulation, gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999). Pour-on or spot-on formulations may be prepared by dissolving the active ingredient in an acceptable liquid carrier vehicle such as butyl digol, liquid paraffin or a non-volatile ester, optionally with the addition of a volatile component such as propan-2-ol. Alternatively, pour-on, spot-on or spray formulations can be prepared by encapsulation, to leave a residue of active agent on the surface of the animal.

Injectable formulations may be prepared in the form of a sterile solution which may contain other substances, for example enough salts or glucose to make the solution isotonic with blood. Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending the active ingredient in the liquid carrier such that the final formulation contains from 0.01 to 10% by weight of the active ingredient. These formulations may be self-preserving, self-sterilising or may be non-sterile to which preservatives may be optionally added.

Equally suitably the components can be administered parenterally, or by injection directly into the blood stream, muscle or into an internal organ. Suitable routes for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as powdered a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents.

Such formulations are prepared in a conventional manner in accordance with standard medicinal or veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are 0.01 to 100 mg per kg of body weight of the animal. Preferably the range is 0.1 to 10 mg per kg.

Formulations may be immediate or be designed to have a controlled or modified release profile. Modified release formulations include those formulations which have a delayed-, sustained-, pulsed-, targeted, or programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298. Alternatively, compounds of the invention may be formulated as a solid, semi-solid, or thixotropic liquid for administration as an implanted depot providing modified release of the active compound. Examples of such formulations include drug-coated stents and PGLA microspheres.

As an alternative the components may be administered to a non-human animal with the feedstuff and for this purpose a concentrated feed additive or premix may be prepared for mixing with the normal animal feed.

For simultaneous administration the two components are combined into a single pharmaceutical composition. The composition may be formulated according to any of the methods described above.

A preferred formulation for treating parasitic infestations in companion animals, including dogs and cats, is a solid dosage form for oral administration. Particularly preferred is a tablet. Tablets may be obtained by compression of a pre-mix comprising the two components and suitable excipients into a single layer, or by compression of two or more premixes so as to give a bilayer tablet wherein each layer may contain only a single component.

Each component may be pre-formulated before inclusion into the mixture for compression. For example, it may be preferable to formulate the arylpyrazole component as a spray-dried dispersion in a suitable matrix before tabletting. Suitable matrices include cellulose derivatives such as hydroxypropylmethylcellulose acetate succinate (HPMCAS).

The invention also relates to a kit comprising two or more separate pharmaceutical compositions, at least one of which contains a compound of formula (I) and one contains a milbemycin or milbemycin derivative, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### EXAMPLES

### A. Preparation of Arylpyrazoles of Formula (I)

The following Examples illustrate the preparation of compounds of the formula (I).

In the following experimental details, nuclear magnetic resonance spectral data were obtained using Varian Inova 300, Varian Inova 400, Varian Mercury 400, Varian Unityplus 400, Bruker AC 300 MHz, Bruker AM 250 MHz or Varian T60 MHz spectrometers, the observed chemical shifts being consistent with the proposed structures. Mass spectral data were obtained on a Finnigan Masslab Navigator, a Fisons Instrument Trio 1000, or a Hewlett Packard GCMS System Model 5971 spectrometer. The calculated and observed ions quoted refer to the isotopic composition of lowest mass. HPLC means high performance liquid chromatography. Room temperature means 20 to 25° C.

Activity for these compounds is reported according to a flea membrane feed test. The assay involves in vitro testing against Ctenocephalides felis conducted according to the following general procedure.

Fleas are cultured in vitro using dog blood. 25-30 adult Ctenocephalides felis (cat flea) were collected and placed in a test chamber (50 ml polystyrene tube with fine nylon mesh sealing the end). Citrated dog blood was prepared by adding aqueous sodium citrate solution (10 ml, 20% w/v, 20 g sodium citrate in 100 ml water) to dog blood (250 ml). Test compounds were dissolved in dimethylsulfoxide to give a working stock solution of 4 mg/ml. The stock solution (12.5 μl) was added to citrated dog blood (5 ml) to give an initial test concentration of 10 μg/ml. For testing at 30 μg/ml, working stock solutions of 12 mg/ml were prepared.

Citrated dog blood containing the test compound (5 ml, 100 μg/ml) was placed into a plastic Petri dish lid, which was kept at 37° C. on a heated pad. Parafilm was stretched over the open top to form a tight membrane for the fleas to feed through. The test chamber containing the fleas was placed carefully onto the parafilm membrane and the fleas commenced feeding.

The fleas were allowed to feed for 2 hours and the test chambers were then removed and stored overnight at room temperature.

The fleas were observed and the percentage of fleas killed recorded. Compounds were initially tested at 100 μg/ml, wherefrom relevant dose responses (100, 30, 10, 3, 1, 0.3, 0.1 μg/ml) were conducted and repeated n=5. Data was plotted to generate ED80, ED90 & ED95 values.

### Example 1

### Cyclopropylmethyl {4-[1-(aminocarbonyl)cyclopropyl]-3-cyano-1-[2,6-dichloro-4-pentafluorothiophenyl]-1H-pyrazol-5-yl}carbamate

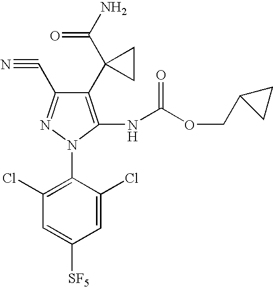

To the compound of Preparation 1 (310 mg, 0.5 mmol) in tetrahydrofuran/water (4:1, 5.2 ml) was added lithium hydroxide monohydrate (218 mg, 5.2 mmol) and the reaction mixture was stirred at room temperature for 24 h. The reaction mixture was acidified with hydrochloric acid (1M) and extracted with ethyl acetate. The combined extracts were washed with water, dried (MgSO₄) and concentrated in vacuo.

To a solution of the residue in tetrahydrofuran (5.20 ml), at 0° C., was added triethylamine (185 ml, 1.3 mmol) and ethyl chloroformate (60 ml, 0.6 mmol). After stirring for 30 min, aqueous ammonium hydroxide solution (3 ml) was added and the reaction mixture was warmed to room temperature. The reaction mixture was adjusted to pH 1 by addition of hydrochloric acid (1M) and extracted with ethyl acetate. The combined extracts were washed with water, dried (MgSO₄) and concentrated in vacuo. The residue was dissolved in acetonitrile (1 ml) and purified by automated preparative liquid chromatography (Gilson system, 150 mm×50 mm LUNA C18 10 mm column) using an acetonitrile:water gradient [50:50 to 95:5]. The appropriate fractions were combined and concentrated to give the title compound (110 mg).

Experimental MH⁺ 560.0; expected 560.0 ¹H-NMR (d₆-DMSO): −0.00-0.04 (2H), 0.24-0.29 (2H), 0.80-0.86 (3H), 1.25-1.29 (2H), 3.65-3.69 (2H), 6.21-6.29 (1H), 6.97-7.03 (1H), 8.33-8.35 (2H), 9.85-9.92 (1H) Flea feed ED₈₀ 0.1 μg/ml

### Example 2

### 1-{5-Amino-3-cyano-1-[2,6-dichloro-4-pentafluorothiophenyl]-1H-pyrazol-4-yl}-cyclopropanecarboxamide

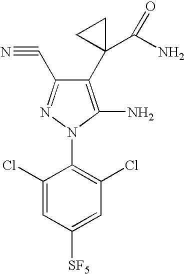

To a solution of the compound of Preparation 10 (615 mg, 1.3 mmol) and triethylamine (204 μl, 1.5 mmol) in tetrahydrofuran (20 ml), at −10° C., was added dropwise ethyl chloroformate (140 μl, 1.5 mmol). The mixture was stirred at 0° C. for 1 h, before addition of ammonium hydroxide (35% in water, 737 μl, 13.3 mmol) in tetrahydrofuran. The reaction mixture was then stirred at 0° C. for 1 h.

To the reaction mixture was added brine and the mixture was extracted with ethyl acetate. The combined extracts were dried (MgSO₄) and concentrated in vacuo to give the crude product. The residue was dissolved in acetonitrile (1 ml) and purified by automated preparative liquid chromatography (Gilson system, 150 mm×50 mm Phenomenex LUNA C18(2) 10 μm column) using an acetonitrile:water gradient [45:55 to 95:5]. The appropriate fractions were concentrated in vacuo to give title compound (95 mg).

Experimental MH⁺ 462.0; expected 462.0 ¹H-NMR (d₆-DMSO): 0.91-0.95 (2H), 1.41-1.46 (2H), 6.12-6.17 (1H), 6.18-6.22 (2H), 7.13-7.18 (1H), 8.39-8.41 (2H) Flea feed ED₈₀ 0.13 μg/ml

### Example 3

### 1-{3-Cyano-1-[2,6-dichloro-4-pentafluorothiophenyl]-5-[(2-fluoroethyl)amino]-1H-pyrazol-4-yl}cyclopropanecarboxamide

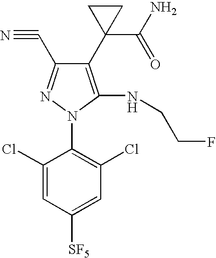

To a solution of the compound of Preparation 11 (150 mg, 0.3 mmol) in tetrahydrofuran (5 ml), at 0° C., was added triethylamine (165 μl, 1.2 mmol), followed by ethyl chloroformate (65 μl, 0.6 mmol). After stirring for 30 min, the mixture was quenched by addition of aqueous ammonium hydroxide solution. The reaction mixture was partitioned between water and ethyl acetate and the two layers were separated. The organic layer was washed with hydrochloric acid (10%) and brine, dried (MgSO₄) and concentrated in vacuo.

The residue was dissolved in acetonitrile/water (9:1, 2 ml) and purified by automated preparative liquid chromatography (Gilson system, 150 mm×30 mm LUNA C18 10 μm column) using an acetonitrile:water gradient [55:45 to 95:5]. The appropriate fractions were combined and concentrated to give the title compound (61 mg).

Experimental MH⁺ 508.1; expected 508.0 1H-NMR (d₆-Acetone): 1.18-1.23 (2H), 1.54-1.60 (2H), 3.58-3.65 (2H), 4.39-4.50 (2H), 5.50-5.61 (1H), 6.30-6.50 (2H), 8.20-8.22 (2H) Flea feed ED₈₀ 0.22 μg/ml

### Example 4

### 1-{5-Amino-3-cyano-1-[2,6-dichloro-4-pentafluorothiophenyl]-1H-pyrazol-4-yl}-2,2-dichlorocyclopropanecarboxamide

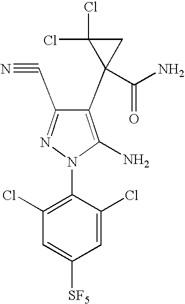

To a solution of the compound of PF-3465372 Preparation 12 (244 mg, 0.5 mmol) in tetrahydrofuran (10 ml), at room temperature and under nitrogen, was added triethylamine (128 μl, 0.9 mmol), followed by ethyl chloroformate (48 mg, 0.5 mmol) in tetrahydrofuran (0.5 ml). After 30 min, ammonium hydroxide (0.27 ml, 2.3 mmol) was added dropwise and the reaction mixture was stirred for 18 h, before being concentrated in vacuo.

The residue was dissolved in acetonitrile (2 ml) with a few drops of dimethyl sulphoxide and purified by automated preparative liquid chromatography (Gilson system, 150 mm×50 mm Sunfire LUNA C18 10 μm column) using an acetonitrile:water [55:45 to 95:5] gradient. The appropriate fractions were combined and concentrated to give the title compound (132 mg).

Experimental MH⁺ 529.9; expected 529.9 Flea feed ED₈₀ 0.55 μg/ml

### Example 5 PF-04135691-00

### Isopropyl {4-[1-(aminocarbonyl)cyclopropyl]-3-cyano-1-[2,6-dichloro-4-pentafluorothiophenyl]-1H-pyrazol-5-yl}carbamate

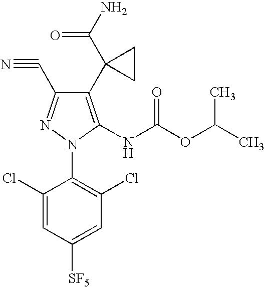

To a solution of the crude compound of PF-04135669-00 Preparation 13 (approx. 0.6 mmol) in tetrahydrofuran (2 ml), at 0° C., was added triethylamine (330 μl, 2.4 mmol) and ethyl chloroformate (120 μl, 1.2 mmol). After stirring for 5 min, aqueous ammonium hydroxide solution (18M, 0.5 ml) was added and the reaction mixture was stirred at room temperature for 18 h. The reaction mixture was adjusted to pH 1 by addition of hydrochloric acid (1M) and extracted with ethyl acetate. The combined extracts were washed with water, dried (MgSO₄) and concentrated under a stream of nitrogen. The residue was dissolved in acetonitrile (1.5 ml) with a few drops of dimethyl sulphoxide and purified by automated preparative liquid chromatography (Gilson system, 150 mm×50 mm Sunfire LUNA C18 10 μm column) using an acetonitrile:water gradient [50:50 to 95:5]. The appropriate fractions were combined and concentrated to give the title compound (143 mg).

Experimental MH⁺ 547.9; expected 548.0 1H-NMR (CD₃OD): 1.09-1.15 (8H), 1.55-1.60 (2H), 4.70-4.80 (1H), 8.19-8.21 (2H) Flea feed ED₈₀ 0.45 μg/ml

### Example 6 PF-3538817

### 1-{3-Cyano-1-[2,6-dichloro-4-pentafluorothiophenyl]-5-(methylamino)-1H-pyrazol-4-yl}-2,2-difluorocyclopropanecarboxamide

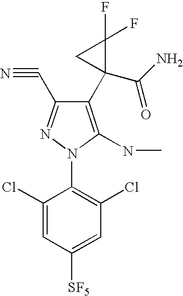

To a solution of the compound of OPreparation 8 PF-3526459 Preparation 20 (977 mg, 1.9 mmol) in tetrahydrofuran (20 ml) was added triethylamine (0.79 ml, 5.7 mmol), followed by ethyl chloroformate (0.2 ml, 2.1 mmol), added dropwise. After stirring for 5 min, ammonium hydroxide (30 wt %, 2.2 ml, 19.1 mmol) was added and the reaction mixture was stirred at room temperature for 30 min.

To the reaction mixture was added hydrochloric acid (2N, 50 ml) and the mixture was extracted with ethyl acetate (3×30 ml). The combined extracts were dried (MgSO₄) and concentrated in vacuo. The residue was dissolved in acetonitrile (3.5 ml) and purified by automated preparative liquid chromatography (Gilson system, 150 mm×50 mm Phenomenex LUNA C18(2) 10 μm column) using an acetonitrile:water [50:50 to 95:5] gradient. The appropriate fractions were combined and concentrated to give the title compound (700 mg).

Experimental MH⁺ 512.2; expected 512.0 ¹H-NMR (d₆DMSO): 1.90-2.01 (1H), 2.75-2.83 (4H), 6.05-6.13 (1H), 7.15-7.22 (1H), 7.59-7.66 (1H), 8.40-8.49 (2H) Flea feed ED₈₀ 0.14 μg/ml

Similarly prepared from the compound of preparation 14 was:

### Example 7 PF-2465753

### 1-{5-Amino-3-cyano-1-[2,6-dichloro-4-pentafluorothiophenyl]-1H-pyrazol-4-yl}-2,2-difluorocyclopropanecarboxamide

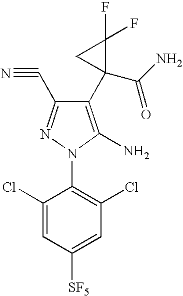

Experimental MH⁺ 497.9; expected 498.0 ¹H-NMR (d₆-DMSO): 1.74-1.84 (1H), 2.51-2.61 (1H), 6.26-6.35 (2H), 7.13-7.22 (1H), 7.44-7.53 (1H), 8.40-8.46 (2H) Flea feed ED₈₀ 0.199 μg/ml

### Example 8 PF-3281087

### 1-[3-Cyano-1-[2,6-dichloro-4-pentafluorothiophenyl]-5-(methylamino)-1H-pyrazol-4-yl]cyclopropanecarboxamide

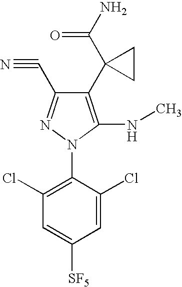

To a solution of the compound of PF-3281082 Preparation 24 (4.7 g, 9.7 mmol) in tetrahydrofuran water (4:1, 100 ml) was added lithium hydroxide monohydrate (4.0 g, 97 mmol). The reaction mixture was stirred at room temperature for 16 h and then adjusted to pH 1 by addition of hydrochloric acid (1M). The mixture was extracted with ethyl acetate and the combined extracts were washed with water, dried (MgSO₄) and concentrated in vacuo.

To a solution of the residue and triethylamine (3.4 ml, 24 mmol) in tetrahydrofuran (100 ml), at 0° C., was added ethyl chloroformate (1.5 ml, 16 mmol). After 20 min at 0° C., the mixture was warmed to room temperature and stirred for 1 h. Anhydrous ammonia (g) was bubbled through the reaction mixture for 15 min, followed by nitrogen for 3 min. The reaction mixture was then partitioned between ethyl acetate and hydrochloric acid (1M) and the organic phase was separated, washed with water, dried (MgSO₄) and concentrated in vacuo.

The residue was dissolved in acetonitrile (1 ml) and purified by automated preparative liquid chromatography (Gilson system, 150 mm×50 mm Phenomenex LUNA C18(2) 10 μm column) using an acetonitrile:water gradient [45:55 to 95:5]. The appropriate fractions were concentrated in vacuo to give title compound (3289 mg).

Experimental MH⁺ 475.9; expected 476.0 ¹H-NMR (CDCl₃): 1.26-1.30 (2H), 1.76-1.81 (2H), 2.88-2.92 (3H), 3.54-3.76 (1H), 5.65-5.75 (2H), 7.91-7.94 (2H) Flea feed ED₈₀ 0.1 μg/ml

### Example 9 PF-03647765-00

### Ethyl {4-[1-(aminocarbonyl)cyclopropyl]-3-cyano-1-[2,6-dichloro-4-pentafluorothiophenyl]-1H-pyrazol-5-yl}carbamate

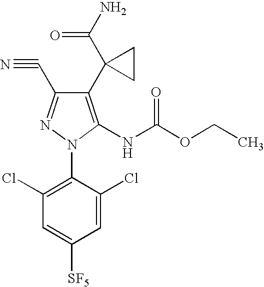

To the compound of PF-03623208-00 Preparation 3 (474 mg, 0.9 mmol) in tetrahydrofuran/water (4:1, 8.6 ml) was added lithium hydroxide monohydrate (360 mg, 8.6 mmol) and the reaction mixture was stirred at room temperature for 24 h.

The reaction mixture was acidified with hydrochloric acid (1M) and extracted with ethyl acetate. The combined extracts were washed with water, dried (MgSO₄) and concentrated in vacuo.

To a solution of the residue in tetrahydrofuran (8.6 ml), at 0° C., was added triethylamine (0.3 ml, 2.2 mmol) and ethyl chloroformate (0.98 ml, 1.0 mmol). After stirring for 30 min, aqueous ammonium hydroxide solution (5 ml) was added and the reaction mixture was warmed to room temperature. The reaction mixture was adjusted to pH 1 by addition of hydrochloric acid (1M) and extracted with ethyl acetate. The combined extracts were washed with water, dried (MgSO₄) and concentrated in vacuo. The residue was dissolved in acetonitrile (1.3 ml) with a few drops of dimethyl sulphoxide and purified by automated preparative liquid chromatography (Gilson system, 150 mm×50 mm LUNA C18(2) 10 μm column) using an acetonitrile:water gradient [45:55 to 95:5]. The appropriate fractions were combined and concentrated to give the title compound (396 mg).

Experimental MH⁺ 534.3; expected 534.0 ¹H-NMR (d₆-DMSO): 0.93-0.97 (2H), 1.03-1.07 (3H), 1.36-1.41 (2H), 3.93-4.01 (2H), 6.40-6.50 (1H), 7.07-7.14 (1H), 8.45-8.47 (2H), 9.92-9.96 (1H) Flea feed ED₈₀ 0.24 μg/ml

### Preparation 1

### Methyl 1-(3-cyano-5-{[(cyclopropylmethoxy)carbonyl]amino}-1-[2,6-dichloro-4-pentafluorothiophenyl]-1H-pyrazol-4-yl)cyclopropanecarboxylate

To a solution of the compound of Preparation 4 (250 mg, 0.5 mmol) and pyridine (0.2 ml, 2.6 mmol) in dichloromethane (5.2 ml), at 0° C., was added phosgene (20% in toluene, 2.7 ml, 5.2 mmol). After stirring at 0° C. for 1 h, cyclopropylmethanol (2 ml) was added and the reaction mixture was stirred at room temperature for 16 h. The reaction mixture was concentrated in vacuo and the residue was partitioned between hydrochloric acid (1M) and ethyl acetate. The organic phase was separated, washed with water, dried (MgSO₄) and concentrated in vacuo to give the title compound (310 mg).

Experimental MH⁺ 575.0; expected 575.0

Similarly Prepared Were

### Preparation 2 PF-3694220

### Methyl 1-{3-cyano-1-[2,6-dichloro-4-pentafluorothiophenyl]-5-[(isopropoxycarbonyl)amino]-1H -pyrazol-4-yl}cyclopropanecarboxylate

from the compound of PF-821386 Preparation 4 and isopropanol.

Experimental MH⁺ 563.0; expected 563.0

### Preparation 3 PF-3623208

### Methyl 1-{3-cyano-1-[2,6-dichloro-4-pentafluorothiophenyl]-5-[(ethoxycarbonyl)amino]-1H -pyrazol-4-yl}cyclopropanecarboxylate

from the compound of PF-821386 Preparation 4 and ethanol

Experimental MH⁺ 549.0; expected 549.0

### Preparation 4

### Methyl 1-{5-amino-3-cyano-1-[2,6-dichloro-4-(pentafluorothiophenyl]-1H-pyrazol-4-yl}cyclopropanecarboxylate

To the compound of Preparation 5 (1.0 g, 1.9 mmol) in 1,4-dioxane (12.5 ml) and methanol (3.5 ml) was added hydrochloric acid (1M, 3.5 ml). The reaction mixture was then heated at reflux overnight. The reaction mixture was concentrated in vacuo and the residue was extracted with ethyl acetate. The combined extracts were washed with water, dried (MgSO₄) and concentrated in vacuo to give the title compound (600 mg).

Experimental MH⁺ 477.0; expected 477.0

### Preparation 5

### Methyl 1-(3-cyano-1-[2,6-dichloro-4-pentafluorothiophenyl]-5-{[(dimethylamino)methylene]-amino}-1H-pyrazol-4-yl)cyclopropanecarboxylate

To trimethylsulphoxonium iodide (892 mg, 4.05 mmol) and sodium hydride (60% in oil, 150 mg, 3.8 mmol) was added dimethyl sulphoxide (20 ml). After stirring for 1 h, the mixture was added to a solution of the compound of Preparation 6 (1.5 g, 2.9 mmol) in dimethyl sulphoxide (20 ml) at 0° C. The reaction mixture was allowed to warm to room temperature and stirred overnight. To the reaction mixture was added hydrochloric acid (1M) and the mixture was extracted with ethyl acetate. The combined organic phases were washed with water, dried (Na₂SO₄) and concentrated in vacuo. The residue was purified on a Biotage column (silica, 100 g) eluting with dichloromethane. The appropriate fractions were combined and concentrated to give the title compound (1.0 g).

Experimental MH⁺ 532.0; expected 532.0

### Preparation 6 PF-803328

### Methyl 2-(3-cyano-1-[2,6-dichloro-4-pentafluorothiophenyl]-5-{[(dimethylamino)methylene]-amino}-1H-pyrazol-4-yl)acrylate

To a solution of the compound of PF-803308 Preparation 7 (3.5 g, 6.5 mmol) in dichloromethane (30 ml) was added triethylamine (5.28 ml, 37.9 mmol) and methanesulphonyl chloride (1.8 ml, 23.5 mmol). The reaction mixture was then stirred at room temperature for 24 h.

To the reaction mixture was added hydrochloric acid (2M) and ice and the mixture was extracted with dichloromethane. The combined extracts were dried (Na₂SO₄) and concentrated in vacuo.

The residue was purified on a Biotage column (silica, 100 g), eluting with dichloromethane. The appropriate fractions were combined and concentrated to give the title compound (1.5 g).

Experimental MH⁺ 518.0; expected 518.0

### Preparation 7 PF-803308

### Methyl 2-(3-cyano-1-[2,6-dichloro-4-pentafluorothiophenyl]-5-{[(dimethylamino)methylene]-amino}-1H-pyrazol-4-yl)-2-hydroxypropanoate

To a solution of the compound of PF-209908 Preparation 8 (3.2 g, 5.6 mmol) in dry tetrahydrofuran (20 ml), at −78° C., was added isopropylmagnesium chloride (2M, 3.1 ml, 6.2 mmol). The mixture was stirred at −78° C. for 30 min and then added to methyl pyruvate (0.76 ml, 8.4 mmol) in tetrahydrofuran (5 ml) at −30° C. The reaction mixture was then stirred overnight at room temperature.

The reaction mixture was acidified with hydrochloric acid (2M) and extracted with ethyl acetate (200 ml). The combined extracts were dried (Na₂SO₄) and concentrated in vacuo to give the title compound (3.5 g).

Experimental MH⁺ 536.0; expected 536.0

### Preparation 8 PF-209908

### N′-{3-cyano-1-[2,6-dichloro-4-pentafluorothiophenyl]-4-iodo-1H-pyrazol-5-yl}-N,N-dimethylimidoformamide

A solution of the compound of UK 529526 Preparation 9 (52 g, 103 mmol) in N,N-dimethylformamide dimethyl acetal (300 ml) was heated at reflux for 5 h, cooled to room temperature and stirred overnight.

The reaction mixture was purified by column chromatography (silica, 1 kg) with gradient elution, hexane:ethyl acetate [6:1 to 4:1]. The appropriate fractions were combined and concentrated to give the title compound (45 g) as a light brown solid.

¹H-NMR (CDCl₃): 2.77-2.81 (3H), 3.02-3.05 (3H), 7.78-7.81 (2H), 8.21-8.24 (1H)

### Preparation 9 UK-529526

### 5-Amino-1-[2,6-dichloro-4-pentafluorothiophenyl]-4-iodo-1H-pyrazole-3-carbonitrile

To a solution of the compound of UK-326020 Preparation 26 (40.0 g, 106 mmol) in acetonitrile (400 ml) was added N-iodosuccinimide (26.4 g, 117 mmol) and the reaction mixture was stirred at room temperature overnight. The reaction mixture was diluted with ethyl acetate (1 l) and washed with aqueous sodium thiosulphate solution (10%, 3×500 ml) and brine (500 ml). The organic phase was dried (MgSO₄) and concentrated in vacuo to give the title compound (53 g) as a brown solid.

¹H-NMR (CDCl₃): 3.87-3.94 (2H), 7.88-7.90 (2H)

### Preparation 10 F-421765

### 1-{5-Amino-3-cyano-1-[2,6-dichloro-4-pentafluorothiophenyl]-1H-pyrazol-4-yl}cyclopropane-carboxylic acid

To a solution of the compound of PF-821386 Preparation 4 (600 mg, 1.3 mmol) in tetrahydrofuran (30 ml) was added lithium hydroxide monohydrate (69 mg, 1.6 mmol). The reaction mixture was then stirred at room temperature for 24 h.

To the reaction mixture was added hydrochloric acid (2M) and the mixture was concentrated in vacuo. The residue was extracted with ethyl acetate and the combined extracts were washed with hydrochloric acid (2M), dried (Na₂SO₄) and concentrated in vacuo to give the title compound (615 mg).

Experimental MH⁺ 462.9; expected 463.0

### Preparation 11 PF-04069325-00

### 1-{3-Cyano-1-[2,6-dichloro-4-pentafluorothiophenyl]-5-[(2-fluoroethyl)amino]-1H-pyrazol-4-yl}-cyclopropanecarboxylic acid

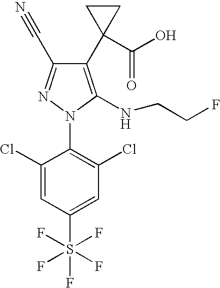

To a solution of the compound of PF-0469122-00 Preparation 15 (100 mg, 0.2 mmol) in tetrahydrofuran (5 ml) was added lithium hydroxide monohydrate (80 mg, 2.0 mmol) in water (1 ml). The reaction mixture was stirred at room temperature for 22 h and then concentrated in vacuo. The residue was partitioned between ethyl acetate and hydrochloric acid (10%) and the organic layer was separated. The aqueous layer was extracted with ethyl acetate and the combined organic phases were dried (MgSO₄) and concentrated in vacuo to give the title compound (100 mg).

Experimental MH⁺ 509.1; expected 509.0

Similarly Prepared Were

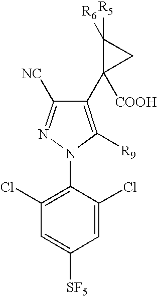

| | | | | From the compound |
|---|---|---|---|---|
| Preparation | R₅ | R₆ | R₉ | of Preparation |
| 12 | Cl | Cl | NH₂ | 16 |
| 13 | H | H | —NHCOOi-Pr | 2 |
| 14 | F | F | NH₂ | 22 |

### Preparation 15 PF-04069122-00

### Methyl 1-{3-cyano-1-[2,6-dichloro-4-pentafluorothiophenyl]-5-[(2-fluoroethyl)amino]-1H-pyrazol-4-yl}cyclopropanecarboxylate

To a solution of the compound of PF-00821386-00 Preparation 4 (250 mg, 0.5 mmol) in dichloromethane (5 ml) was added 2-fluoroethanol (160 mg, 2.5 mmol), followed by Dess-Martin periodinane (1.15 g, 2.5 mmol). After stirring at room temperature for 5 h, the solution was filtered through Celite® and the filtrate was added carefully to a solution of sodium borohydride (200 mg, 5.0 mmol) in methanol (5 ml) at 0° C. The reaction mixture was stirred at 0° C. for 30 min, before addition of water and ethyl acetate, and the organic layer was separated. The aqueous layer was extracted with ethyl acetate and the combined organic phases were washed with aqueous sodium hydrogen carbonate solution and brine, dried (MgSO₄) and concentrated in vacuo to give the title compound (100 mg).

1H-NMR (CDCl₃): 1.35-1.40 (2H), 1.71-1.78 (2H), 3.20-3.32 (2H), 3.65-3.67 (3H), 4.30-4.45 (2H), 7.90-7.94 (2H)

### Preparation 16 PF-3465371

### Ethyl 1-{5-amino-3-cyano-1-[2,6-dichloro-4-pentafluorothiophenyl]-1H-pyrazol-4-yl}-2,2-dichlorocyclopropanecarboxylate

To a solution of the compound of UK-326019 Preparation 27 (1.0 g, 3.5 mmol) in ethanol (5 ml), at 0° C., was added tetrafluoroboric acid (48% in water, 1.0 ml, 7.4 mmol), followed by isoamyl nitrite (0.32 ml, 3.9 mmol). The reaction mixture was then stirred for 40 min. The product was collected by filtration and dried to give 2,6-dichloro-4-pentafluorothiobenzenediazonium tetrafluoroborate A solution of PF-3458584 Preparation 17 (100 mg, 0.3 mmol) and pyridine (75 μl) in methanol (2 ml), at 0° C., was stirred for 15 min, before addition of 2,6-dichloro-4-pentafluorothiobenzenediazonium tetrafluoroborate (121 mg, 0.3 mmol). The reaction mixture was then stirred at room temperature for 30 min. To the reaction mixture was added diethyl ether (20 ml) and the solution was washed with water and brine. The organic layer was separated, dried (Na₂SO₄) and concentrated in vacuo to give the title compound (220 mg).

Experimental MH⁺ 558.8; expected 558.9

### Preparation 17 PF-3458584

### Ethyl 2,2-dichloro-1-(1,2-dicyano-3-methoxy-3-oxopropyl)cyclopropane-carboxylate

To a solution of the compound of PF-3458898 Preparation 18 (1.0 g, 3.4 mmol) in methanol (15 ml), at 0° C. and under nitrogen, was added potassium cyanide (267 mg, 4.1 mmol) and the reaction mixture was stirred for 1 h. Glacial acetic acid (390 μl) and silica (1.00 g) were added and the mixture was concentrated in vacuo.

The product/silica mix was purified by column chromatography (silica) with gradient elution, diethyl ether:cyclohexane [3:7 to 1:1]. The appropriate fractions were combined and concentrated to give the title compound (440 mg).

¹H-NMR (CDCl₃): 1.39-1.41 (3H), 1.65-2.00 (1H), 2.42-2.70 (1H), 3.32-3.41 (1H), 3.89-3.99 (3H), 4.21-4.27 (1H), 4.35-4.42 (2H)

### Preparation 18 PF-3458898

### Ethyl 2,2-dichloro-1-[2-cyano-3-methoxy-3-oxoprop-1-en-1-yl]-cyclopropanecarboxylate

A mixture of the compound of PF-3458582 Preparation 19 (8.6 g, 40.0 mmol), methyl cyanoacetate (3.5 ml, 40.0 mmol) and piperidine (1.2 ml, 12.0 mmol) in acetic acid (30 ml) was heated at reflux, under nitrogen, for 60 h. The reaction mixture was poured into water (500 ml) and extracted with dichloromethane (2×150 ml). The combined extracts were washed with saturated aqueous sodium hydrogencarbonate solution (200 ml), dried (Na₂SO₄) and concentrated in vacuo.

The residue was purified by column chromatography (silica), eluting with diethyl ether:cyclohexane [2:8]. The appropriate fractions were combined and concentrated to give the title compound (6.00 g). ¹H-NMR (CDCl₃): 1.19-1.28 (3H), 2.25-2.30 (1H), 2.81-2.85 (1H), 3.91-3.94 (3H), 4.29-4.41 (2H), 7.89-7.92 (1H)

### Preparation 19 PF-3455852

### Ethyl 2,2-dichloro-1-formylcyclopropanecarboxylate

A solution of the compound of PF-3458580 Preparation 25 (5.0 g, 19.7 mmol) in dichloromethane (50 ml) was purged with nitrogen and cooled to −78° C. To the solution was added dropwise diisobutylaluminium hydride (1M in dichloromethane, 39.4 ml, 39.4 mmol), ensuring that the temperature did not rise above −65° C. After stirring at this temperature for 2 h, saturated aqueous ammonium chloride solution was added, followed by hydrochloric acid (2N, 5 ml), and the mixture was allowed to warm to room temperature.

The reaction mixture was filtered, washed with brine, dried (Na₂SO₄) and concentrated in vacuo. The residue was purified by column chromatography (silica) eluting with diethyl ether/cyclohexane [2:8]. The appropriate fractions were combined and concentrated to give the title compound (900 mg). ¹H-NMR (CDCl₃): 1.35-1.38 (3H), 2.40-2.50 (2H), 4.31-4.39 (2H), 9.96-9.99 (1H)

### Preparation 20 PF-3526459

### 1-[3-Cyano-1-[2,6-dichloro-4-pentafluorothiophenyl]-5-(methylamino)-1H-pyrazol-4-yl]-2,2-difluorocyclopropanecarboxylic acid

A mixture of the compound of PF-3475559 Preparation 21 (960 mg, 1.8 mmol) and lithium hydroxide monohydrate (383 mg, 9.1 mmol) in tetrahydrofuran (30 ml) and water (10 ml) was stirred at room temperature for 18 h. The reaction mixture was concentrated in vacuo and the residue was partitioned between hydrochloric acid (2N, 50 ml) and ethyl acetate (50 ml). The organic layer was separated, washed with brine (50 ml), dried (MgSO₄) and concentrated in vacuo to give the title compound (977 mg).

Experimental MH⁺ 512.9; expected 513.0

### Preparation 21 PF-3475559

### Methyl 1-{3-cyano-1-[2,6-dichloro-4-pentafluorothiophenyl]-5-(methylamino)-1H-pyrazol-4-yl}-2,2-difluorocyclopropanecarboxylate

To a solution of the compound of Preparation 22 (243 mg, 0.5 mmol) in 1,4-dioxane (6 ml) was added trimethyl orthoformate (0.2 ml, 1.9 mmol) and p-toluenesulphonic acid (2 mg). The reaction mixture was heated at 60° C. for 4 h and then stirred at room temperature for 18 h. The mixture was concentrated in vacuo and to the residue was added toluene (10 ml). The solution was concentrated in vacuo and to the residue was added acetic acid (5 ml) and sodium cyanoborohydride (60 mg, 1.0 mmol). The reaction mixture was stirred at room temperature for 2 h and then concentrated in vacuo. The residue was partitioned between water (20 ml) and ethyl acetate (20 ml) and the organic phase was separated, dried (MgSO₄) and concentrated in vacuo to give the title compounnd (200 mg).

Experimental MH⁺ 526.9; expected 527.0

### Preparation 22 PF-2455319

### Methyl 1-{5-amino-3-cyano-1-[2,6-dichloro-4-pentafluorothiophenyl]-1H-pyrazol-4-yl}-2,2-difluorocyclopropanecarboxylate

A solution of the compound of PF-2455223 Preparation 23 (3.0 g, 5.3 mmol) in p-toluenesulphonic acid (10% in methanol, 80 ml) was heated at reflux for 18 h. The reaction mixture was concentrated in vacuo and the residue was partitioned between saturated aqueous sodium hydrogencarbonate solution and ethyl acetate. The organic phase was separated, washed with brine, dried (MgSO₄) and concentrated in vacuo.

The residue was triturated with cold ethanol to give the title compound (500 mg).

Experimental MH⁺ 513.0; expected 513.0

### Preparation 23 PF-2455223

### Methyl 1-(3-cyano-1-[2,6-dichloro-4-pentafluorothiophenyl]-5-{[(dimethylamino)methylene]amino}-1H-pyrazol-4-yl)-2,2-difluorocyclopropane-carboxylate

To a mixture of the compound of PF-803328 Preparation 6 (2.2 g, 4.3 mmol) and sodium fluoride (3 mg) in toluene (5.4 ml) at reflux was added trimethylsilyl-2,2-difluoro-2-(fluorosulphonyl)acetate (3.4 ml, 17.3 mmol), via syringe. After heating at reflux for 4 h, the reaction mixture was cooled to room temperature and stirred for 16 h.

The reaction mixture was concentrated in vacuo and the residue was purified by column chromatography (silica) with gradient elution, ethyl acetate:hexane [10:90 to 35:65]. The appropriate fractions were combined and concentrated to give the title compound (2.0 g).

Experimental MH⁺ 568.1; expected 568.0

### Preparation 24 PF-3281082

### Methyl 1-[3-cyano-1-[2,6-dichloro-4-pentafluorothiophenyl]-5-(methylamino)-1H-pyrazol-4-yl]cyclopropanecarboxylate

To a solution of the compound of PF-821386 Preparation 4 (6.8 g, 14.3 mmol) in triethylorthoformate (180 ml) was added hydrochloric acid (concentrated, 0.5 ml) and the reaction mixture was heated at 50° C. for 2 h. The mixture was concentrated in vacuo and to the residue was added ethanol (120 ml). The solution was cooled to 0° C. and sodium borohydride (1.2 g, 31.5 mmol) was added over 5 min. After stirring for 16 h at room temperature, acetic acid (2.5 ml) was added, followed by water (300 ml). After a further 10 min, the mixture was extracted with ethyl acetate and the combined extracts were dried (MgSO₄) and concentrated in vacuo.

The residue was purified by column chromatography (silica), eluting with ethyl acetate/hexane [1:3]. The appropriate fractions were combined and concentrated to give the title compound (4.74 g).

Experimental MH⁺ 491.0; expected 491.0

### Preparation 25 PF-3458580

### Diethyl 2,2-dichlorocyclopropane-1,1-dicarboxylate

Reference: Synthetic Communications (1989), 19(1-2), 141-6.

### Preparation 26 UK-326020

### 5-Amino-1-[2,6-dichloro-4-pentafluorothiophenyl]-1H-pyrazole-3-carbonitrile

Reference: WO 9306089 A1, EP 605469 A1

### Preparation 27 UK-326019

### 2,6-Dichloro-4-pentafluorothioaniline

Reference: WO 9306089 A1

### B. Preparation of Milbemycin Oxime

Milbemycin oxime may be prepared from a mixture of milbemycin A3 and A4 according to the methods set out in EP 110667 and U.S. Pat. No. 4,547,520.

### C. Activity Following Oral Dosage Formulation

The compound of Example 1 was formulated as a spray dried dispersion in the polymer HPMCAS-HG at 25% active ingredient and co-formulated by addition and mixing with 50%:50% w/w, a blended inert excipient mixture containing microcrystalline methylcellulose (70% w/w and sodium starch glycolate 30% w/w). Each capsule was filled to deliver an accurate dose of the test composition according to dog weight the day prior to treatment. Milbemycin oxime was added to the capsule to deliver an accurate dose of 0.5 mg/kg, according to dog weight and on completion of filling the test composition. All capsule contents were thoroughly mixed prior to administration.

### Evaluation of the Target Animal Efficacy of the Compound of Example 1

In this evaluation 30 dogs were each infested with approximately 100 unfed adult fleas (Ctenocephalides felis), 50 adult brown dog ticks (Rhipicephalus sanguineus) and 50 adult American dog ticks (Dermacentor variabilis).

Each dog was assessed for its ability to retain brown dog tick and flea infestation by examination by combing and removal 48 h post-infestation. The dogs were blocked by tick count and randomly assigned to one of 5 treatment groups. Two days prior to treatment each dog was infested with, 50 adult brown dog ticks, 50 adult American dog ticks and approximately 100 unfed fleas. The test composition was administered orally at 2.0, 4.0 or 6.0 mg/kg body weight in combination with milbemycin oxime at a constant dose-rate of 0.5 mg/kg via a single solid filled capsule. The remaining 2 groups of dogs received no treatment or received treatment of the commercial product Frontline™ Plus. At one-day post treatment, the live ticks and fleas were counted on all dogs to check for knockdown efficacy. At two days post treatment, each dog was examined and combed to count and remove live ticks and fleas. The dogs were subsequently re-infested with both tick species and fleas and examined and comb counted at weekly intervals. Efficacy of the test composition was determined relative to the untreated dogs, and is recorded as a percentage of the geometric mean of the ectoparasite counts for the untreated control animals. The data are shown in tables 1, 2 & 3 with comparison of efficacy made against the commercial product Frontline™ Plus.

TABLE 1
Percentage Efficacy of Compound of Example 1 plus Milbemycin
oxime (0.5 mg/kg) against Adult Fleas (Ctenocephalides felis).

| Treatment | | | | | | | |
|---|---|---|---|---|---|---|---|
| Dose | | | | | | | |
| Compound of | | | | | | | |
| Example 1/mg/kg | Day 1 | Day 2 | Day 9 | Day 16 | Day 23 | Day 30 | Day 37 |
| 2.0 | 100 | 100 | 100 | 99.9 | 100 | 100 | 100 |
| 4.0 | 100 | 99.6 | 100 | 100 | 100 | 100 | 100 |
| 6.0 | 100 | 100 | 99.9 | 100 | 100 | 100 | 100 |
| Frontline ™ | 93.8 | 99.4 | 100 | 100 | 100 | 100 | 99.9 |
| Plus | | | | | | | |

TABLE 2
Percentage Efficacy of Composition 6033 plus Milbemycin
oxime (0.5 mg/kg) against adult brown dog ticks
(Rhipicephalus sanguineus).

| Treatment | | | | | | | |
|---|---|---|---|---|---|---|---|
| Dose | | | | | | | |
| Compound of | | | | | | | |
| Example 1/mg/kg | Day 1 | Day 2 | Day 9 | Day 16 | Day 23 | Day 30 | Day 37 |
| 2.0 | 83.4 | 98.6 | 92.9 | 96.6 | 96.3 | 92.3 | 73.5 |
| 4.0 | 94.4 | 98.2 | 95.7 | 99.5 | 97.5 | 94.6 | 87.9 |
| 6.0 | 99.3 | 99.3 | 95.2 | 98.9 | 97.5 | 94.8 | 95.6 |
| Frontline ™ | 70.5 | 79.2 | 99.4 | 98.9 | 83.8 | 81.3 | 53.7 |
| Plus | | | | | | | |

TABLE 3
Percentage Efficacy of Composition 6033 plus Milbemycin
oxime (0.5 mg/kg) against adult American dog ticks
(Dermacentor variabilis).

| Treatment | | | | | | | |
|---|---|---|---|---|---|---|---|
| Dose | | | | | | | |
| Compound of | | | | | | | |
| Example 1/mg/kg | Day 1 | Day 2 | Day 9 | Day 16 | Day 23 | Day 30 | Day 37 |
| 2.0 | 34.7 | 94.9 | 92.9 | 98.1 | 96.8 | 90.7 | 89.3 |
| 4.0 | 72.9 | 98.7 | 99.1 | 99.7 | 98.6 | 97 | 96.9 |
| 6.0 | 88 | 100 | 98.5 | 99.4 | 99.3 | 100 | 99.7 |
| Frontline ™ | 70.3 | 86.3 | 100 | 99.4 | 99.7 | 95 | 94.2 |
| Plus | | | | | | | |

As shown by the data in tables 1-3 the composition of the invention is stable and efficacious over an extended period of time.

1. A method of treating a parasitic infestation in a host animal, comprising simultaneously, sequentially or separately administering to said host animal:
a) a therapeutically effective amount of a compound according to formula (I) 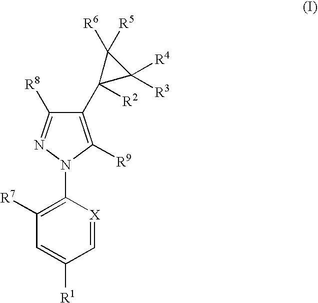
wherein:
X is selected from CR¹⁰ or N;
R¹ is selected from halo, cyano, hydroxy, C₁₋₆ alkyl, C₁₋₆ alkoxy, C₁₋₆ alkanoyl, C₁₋₆ haloalkyl, C₁₋₆ haloalkoxy, C₁₋₆ haloalkanoyl, amino, C₁₋₆ alkyl amino, di C₁₋₆ alkyl amino, het, phenyl, SF₅ and S(O)ₙR¹¹;
R² is selected from cyano, hydroxy, C(O)OH, het, phenyl, S(O)ₙR¹¹, C(O)NR^{a}R^{b} and C(S)NR^{a}R^{b};
or R² is selected from C₃₋₈ cycloalkyl, C₃₋₈ cycloalkylC₁₋₆ alkyl, C₂₋₆ alkenyl, C₂₋₆ alkynyl, C₁₋₆ alkanoyl, C(O)OC₁₋₆ alkyl, amino, C₁₋₆ alkyl amino, and di C₁₋₆ alkyl amino each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{a} and R^{b} are independently selected from hydrogen, het, phenyl, and S(O)ₙR¹¹;
or either one or both of R^{a} and R^{b} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, and C(O)OC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{a} and R^{b} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R² and R^{e} together with the N atom to which R^{e} is attached may form a six to seven-membered saturated, partially saturated, or unsaturated heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R³, R⁴, R⁵ and R⁶ are independently selected from hydrogen, halo, cyano, hydroxy, C(O)OH, nitro, phenyl, and S(O)ₙR¹¹;
or either one or more of R³, R⁴, R⁵ and R⁶ are independently selected from C₁₋₄ alkyl, C(O)NR^{c}R^{d}, C(S)NR^{c}R^{d}, C₁₋₄ alkoxy, C₁₋₄ alkanoyl, C(O)OC₁₋₄ alkyl, amino which R³, R⁴, R⁵ and R⁶ may be optionally and independently further substituted by one or more substituents selected from, where chemically possible, cyano, nitro, halo, hydroxy, C₁₋₄ alkyl and amino;
and where not more than two of R³, R⁴, R⁵ and R⁶ are selected from cyano, hydroxy, C(O)OH, nitro, phenyl, S(O)ₙR¹¹, C(O)NR^{c}R^{d}, C(S)NR^{c}R^{d}, C₁₋₄ alkoxy, C₁₋₄ alkanoyl, C(O)OC₁₋₄ alkyl, and amino;
R⁷ is selected from halo, C₁₋₆ alkyl and C₁₋₆ alkoxy where, when R⁷ is C₁₋₆ alkyl or C₁₋₆ alkoxy, R⁷ may be optionally substituted with one or more halo substituents;
R⁸ is selected from hydrogen, cyano, hydroxy, C(O)OH, nitro, halo, het, phenyl and S(O)ₙR¹¹;
or R⁸ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₁₋₆ alkanoyl, and C(O)OC₁₋₆ alkyl, which R⁸ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁸ is amino, which R⁸ may be optionally and independently further substituted by one or more substituents selected from, where chemically possible, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, het, phenyl and S(O)ₙR¹¹;
R⁹ is selected from hydrogen, halo, cyano, hydroxy, C(O)OH, nitro, het, phenyl, S(O)ₙR¹¹ and NR^{e}R^{f};
or R⁹ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₃₋₈ cycloalkylC₁₋₆ alkoxy, C₁₋₆ alkanoyl, C(O)OC₁₋₆ alkyl, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{e} and R^{f} are independently selected from hydrogen, het, phenyl and S(O)ₙR¹¹;
or either one or both of R^{e} and R^{f} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R^{f} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R² together with the atoms to which they are attached may form a six to seven-membered heterocyclic ring as previously described;
R¹⁰ is selected from halo, C₁₋₆ alkyl and C₁₋₆ alkoxy and where when R¹⁰ is C₁₋₆ alkyl or C₁₋₆ alkoxy it may optionally be substituted with one or more halo substituents;
each of R^{c} and R^{d} are independently selected from hydrogen, C₁₋₆ alkyl, C₂₋₆ alkenyl, C₃₋₈ cycloalkyl, C₃₋₈ cycloalkylC₁₋₆ alkyl, C₁₋₆ haloalkyl, C₃₋₈ cycloalkylC₁₋₆ haloalkyl, C₁₋₆ alkanoyl, C₁₋₆ haloalkanoyl, C(O)OC₁₋₆ alkyl, het, phenyl and S(O)ₙR¹¹;
or R^{c} and R^{d} together with the N atom to which at least one of them is attached may form a three to seven-membered saturated, partially saturated, unsaturated or aromatic heterocyclic ring which may optionally contain one or more further N, O or S atoms;
each n is independently 0, 1 or 2;
each R¹¹ is independently selected from hydrogen, hydroxy, C₁₋₆ alkyl, C₁₋₆ haloalkyl, amino, C₁₋₆ alkyl amino and di C₁₋₆ alkyl amino;
each phenyl may be optionally substituted by one or more further substitutents selected from the group consisting of halo, cyano, nitro, hydroxy, C₁₋₆ alkyl, C₁₋₆ haloalkyl, C₁₋₆ alkoxy, C₁₋₆ haloalkoxy, amino, C₁₋₆ alkyl amino, di C₁₋₆ alkyl amino, —NHS(O)ₙR¹¹, and S(O)ₙR¹¹;
and each het independently represents a four to seven membered heterocyclic ring, which is aromatic or non-aromatic, unsaturated, partially saturated or saturated and which contains one or more heteroatoms selected from nitrogen, N-oxide, oxygen, sulphur and wherein said heterocyclic ring is optionally substituted, where the valence allows, with one or more substituents selected from halo, cyano, nitro, C₁₋₆ alkyl, C₁₋₆ haloalkyl, C₁₋₆ alkoxy, OC(O) C₁₋₆ alkyl, C₁₋₆ alkanoyl, C(O)O C₁₋₆ alkyl and NR^{g}R^{h}, where R^{g} and R^{h} are independently selected from hydrogen, C₁₋₆ alkyl and C₂₋₆ alkenyl, and where each of the above groups may include one or more optional substituents where chemically possible independently selected from cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, C₁₋₆ alkyl amino, di C₁₋₆ alkyl amino, phenyl and S(O)ₙR¹¹;
or a pharmaceutically acceptable salt or a prodrug thereof; and
b) a therapeutically effective amount of a second antiparasitic agent.

2. The method according to claim 1 wherein the second antiparasitic agent is an anthelmintic agent.

3. The method according to claim 2 wherein the anthelmintic agent is a macrocyclic lactone.

4. The method according to claim 3 wherein the macrocyclic lactone anthelmintic agent is a milbemycin or a derivative thereof.

5. The method according to claim 4 wherein the milbemycin or derivative thereof is milbemycin oxime.

6. The method according to claim 1 wherein the compound according to formula (I) is selected from:
cyclopropylmethyl {4-[1-(aminocarbonyl)cyclopropyl]-3-cyano-1-[2,6-dichloro-4-pentafluorothiophenyl]-1H-pyrazol-5-yl}carbamate;
1-{5-amino-3-cyano-1-[2,6-dichloro-4-pentafluorothiophenyl]-1H-pyrazol-4-yl}-cyclopropanecarboxamide;
1-{3-cyano-1-[2,6-dichloro-4-pentafluorothiophenyl]-5-[(2-fluoroethyl)amino]-1H-pyrazol-4-yl}cyclopropanecarboxamide;
1-{5-amino-3-cyano-1-[2,6-dichloro-4-pentafluorothiophenyl]-1H-pyrazol-4-yl}-2,2-dichlorocyclopropanecarboxamide;
isopropyl {4-[1-(aminocarbonyl)cyclopropyl]-3-cyano-1-[2,6-dichloro-4-pentafluorothiophenyl]-1H-pyrazol-5-yl}carbamate;
1-{3-cyano-1-[2,6-dichloro-4-pentafluorothiophenyl]-5-(methylamino)-1H-pyrazol-4-yl}-2,2-difluorocyclopropanecarboxamide;
1-{5-amino-3-cyano-1-[2,6-dichloro-4-pentafluorothiophenyl]-1H-pyrazol-4-yl}-2,2-difluorocyclopropanecarboxamide;
1-[3-cyano-1-[2,6-dichloro-4-pentafluorothiophenyl]-5-(methylamino)-1H-pyrazol-4-yl]cyclopropanecarboxamide; and
ethyl {4-[1-(aminocarbonyl)cyclopropyl]-3-cyano-1-[2,6-dichloro-4-pentafluorothiophenyl]-1H-pyrazol-5-yl}carbamate.

7. The method according to claim 6 wherein the second antiparasitic agent is an anthelmintic agent.

8. The method according to claim 7 wherein the anthelmintic agent is a macrocyclic lactone.

9. The method according to claim 8 wherein the macrocyclic lactone anthelmintic agent is a milbemycin or a derivative thereof.

10. The method according to claim 9 wherein the milbemycin or derivative thereof is milbemycin oxime.

11. The method according to claim 1 wherein the compound according to formula (I) is cyclopropylmethyl {4-[1-(aminocarbonyl)cyclopropyl]-3-cyano-1-[2,6-dichloro-4-pentafluorothio-phenyl]-1H-pyrazol-5-yl}carbamate.

12. The method according to claim 11 wherein the second antiparasitic agent is an anthelmintic agent.

13. The method according to claim 12 wherein the anthelmintic agent is a macrocyclic lactone.

14. The method according to claim 13 wherein the macrocyclic lactone anthelmintic agent is a milbemycin or a derivative thereof.

15. The method according to claim 14 wherein the milbemycin or derivative thereof is milbemycin oxime.

16. The method according to any one of claims 1 to 15 wherein the host animal is a mammal.

17. The method according to claim 16 wherein the host animal is a human.

18. The method according to claim 16 wherein the host animal is a non-human mammal.

19. The method according to claim 18 wherein the non-human mammal is selected from dogs, cats, horses, cattle, sheep and pigs.

20. The method according to any one of claims 1 to 15 wherein the host animal is a bird or a fish.

21. The method according to any one of claims 1 to 15 wherein the compound of formula (I), or pharmaceutically acceptable salt or a prodrug thereof, and the second antiparasitic agent, are administered together in a single pharmaceutical composition.

22. The method according to claim 21 wherein the single pharmaceutical composition is adapted for oral administration.

23. A pharmaceutical composition comprising:
a) a compound according to formula (I) 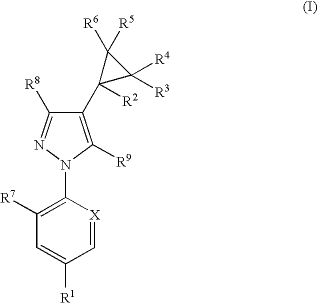
wherein:
X is selected from CR¹⁰ or N;
R¹ is selected from halo, cyano, hydroxy, C₁₋₆ alkyl, C₁₋₆ alkoxy, C₁₋₆ alkanoyl, C₁₋₆ haloalkyl, C₁₋₆ haloalkoxy, C₁₋₆ haloalkanoyl, amino, C₁₋₆ alkyl amino, di C₁₋₆ alkyl amino, het, phenyl, SF₅ and S(O)ₙR¹¹;
R² is selected from cyano, hydroxy, C(O)OH, het, phenyl, S(O)ₙR¹¹, C(O)NR^{a}R^{b} and C(S)NR^{a}R^{b};
or R² is selected from C₃₋₈ cycloalkyl, C₃₋₈ cycloalkylC₁₋₆ alkyl, C₂₋₆ alkenyl, C₂₋₆ alkynyl, C₁₋₆ alkanoyl, C(O)OC₁₋₆ alkyl, amino, C₁₋₆ alkyl amino, and di C₁₋₆ alkyl amino each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{a} and R^{b} are independently selected from hydrogen, het, phenyl, and S(O)ₙR¹¹;
or either one or both of R^{a} and R^{b} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, and C(O)OC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{a} and R^{b} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R² and R^{e} together with the N atom to which R^{e} is attached may form a six to seven-membered saturated, partially saturated, or unsaturated heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R³, R⁴, R⁵ and R⁶ are independently selected from hydrogen, halo, cyano, hydroxy, C(O)OH, nitro, phenyl, and S(O)ₙR¹¹;
or either one or more of R³, R⁴, R⁵ and R⁶ are independently selected from C₁₋₄ alkyl, C(O)NR^{c}R^{d}, C(S)NR^{c}R^{d}, C₁₋₄ alkoxy, C₁₋₄ alkanoyl, C(O)OC₁₋₄ alkyl, amino which R³, R⁴, R⁵ and R⁶ may be optionally and independently further substituted by one or more substituents selected from, where chemically possible, cyano, nitro, halo, hydroxy, C₁₋₄ alkyl and amino;
and where not more than two of R³, R⁴, R⁵ and R⁶ are selected from cyano, hydroxy, C(O)OH, nitro, phenyl, S(O)ₙR¹¹, C(O)NR^{c}R^{d}, C(S)NR^{c}R^{d}, C₁₋₄ alkoxy, C₁₋₄ alkanoyl, C(O)OC₁₋₄ alkyl, and amino;
R⁷ is selected from halo, C₁₋₆ alkyl and C₁₋₆ alkoxy where, when R⁷ is C₁₋₆ alkyl or C₁₋₆ alkoxy, R⁷ may be optionally substituted with one or more halo substituents;
R⁸ is selected from hydrogen, cyano, hydroxy, C(O)OH, nitro, halo, het, phenyl and S(O)ₙR¹¹;
or R⁸ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₁₋₆ alkanoyl, and C(O)OC₁₋₆ alkyl, which R⁸ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁸ is amino, which R⁸ may be optionally and independently further substituted by one or more substituents selected from, where chemically possible, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, het, phenyl and S(O)ₙR¹¹;
R⁹ is selected from hydrogen, halo, cyano, hydroxy, C(O)OH, nitro, het, phenyl, S(O)ₙR¹¹ and NR^{e}R^{f};
or R⁹ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₃₋₈ cycloalkylC₁₋₆ alkoxy, C₁₋₆ alkanoyl, C(O)OC₁₋₆ alkyl, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{e} and R^{f} are independently selected from hydrogen, het, phenyl and S(O)ₙR¹¹;
or either one or both of R^{e} and R^{f} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R^{f} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R² together with the atoms to which they are attached may form a six to seven-membered heterocyclic ring as previously described;
R¹⁰ is selected from halo, C₁₋₆ alkyl and C₁₋₆ alkoxy and where when R¹⁰ is C₁₋₆ alkyl or C₁₋₆ alkoxy it may optionally be substituted with one or more halo substituents;
each of R^{c} and R^{d} are independently selected from hydrogen, C₁₋₆ alkyl, C₂₋₆ alkenyl, C₃₋₈ cycloalkyl, C₃₋₈ cycloalkylC₁₋₆ alkyl, C₁₋₆ haloalkyl, C₃₋₈ cycloalkylC₁₋₆ haloalkyl, C₁₋₆ alkanoyl, C₁₋₆ haloalkanoyl, C(O)OC₁₋₆ alkyl, het, phenyl and S(O)ₙR¹¹;
or R^{c} and R^{d} together with the N atom to which at least one of them is attached may form a three to seven-membered saturated, partially saturated, unsaturated or aromatic heterocyclic ring which may optionally contain one or more further N, O or S atoms;
each n is independently 0, 1 or 2;
each R¹¹ is independently selected from hydrogen, hydroxy, C₁₋₆ alkyl, C₁₋₆ haloalkyl, amino, C₁₋₆ alkyl amino and di C₁₋₆ alkyl amino;
each phenyl may be optionally substituted by one or more further substitutents selected from the group consisting of halo, cyano, nitro, hydroxy, C₁₋₆ alkyl, C₁₋₆ haloalkyl, C₁₋₆ alkoxy, C₁₋₆ haloalkoxy, amino, C₁₋₆ alkyl amino, di C₁₋₆ alkyl amino, —NHS(O)ₙR¹¹, and S(O)ₙR¹¹;
and each het independently represents a four to seven membered heterocyclic ring, which is aromatic or non-aromatic, unsaturated, partially saturated or saturated and which contains one or more heteroatoms selected from nitrogen, N-oxide, oxygen, sulphur and wherein said heterocyclic ring is optionally substituted, where the valence allows, with one or more substituents selected from halo, cyano, nitro, C₁₋₆ alkyl, C₁₋₆ haloalkyl, C₁₋₆ alkoxy, OC(O) C₁₋₆ alkyl, C₁₋₆ alkanoyl, C(O)O C₁₋₆ alkyl and NR^{g}R^{h}, where R^{g} and R^{h} are independently selected from hydrogen, C₁₋₆ alkyl and C₂₋₆ alkenyl, and where each of the above groups may include one or more optional substituents where chemically possible independently selected from cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, C₁₋₆ alkyl amino, di C₁₋₆ alkyl amino, phenyl and S(O)ₙR¹¹;
or a pharmaceutically acceptable salt or a prodrug thereof; and
b) a second antiparasitic agent.

24. The pharmaceutical composition according to claim 23 wherein the second antiparasitic agent is an anthelmintic agent.

25. The pharmaceutical composition according to claim 24 wherein the anthelmintic agent is a macrocyclic lactone.

26. The pharmaceutical composition according to claim 25 wherein the macrocyclic lactone is a milbemycin or a derivative thereof.

27. The pharmaceutical composition according to claim 26 wherein the milbemycin or derivative thereof is milbemycin oxime.

28. The pharmaceutical composition according to claim 23 wherein the compound according to formula (i) is cyclopropylmethyl {4-[1-(aminocarbonyl)cyclopropyl]-3-cyano-1-[2,6-dichloro-4-pentafluorothiophenyl]-1H-pyrazol-5-yl}carbamate.

29. The pharmaceutical composition according to claim 28 wherein the second antiparasitic agent is an anthelmintic agent.

30. The pharmaceutical composition according to claim 29 wherein the anthelmintic agent is a macrocyclic lactone.

31. The pharmaceutical composition according to claim 30 wherein the macrocyclic lactone is a milbemycin or a derivative thereof.

32. The pharmaceutical composition according to claim 31 wherein the milbemycin or derivative thereof is milbemycin oxime.

33. The pharmaceutical composition according to any one of claims 23 to 32 which is adapted for oral administration.

34. A kit for treating a parasitic infestation in a host animal, comprising:
a) a pharmaceutical composition comprising a therapeutically effective amount of a compound according to formula (I) 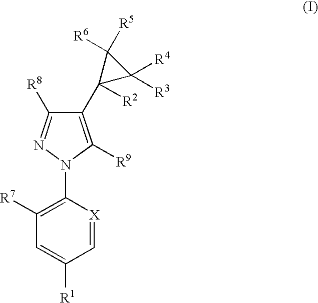
wherein:
X is selected from CR¹⁰ or N;
R¹ is selected from halo, cyano, hydroxy, C₁₋₆ alkyl, C₁₋₆ alkoxy, C₁₋₆ alkanoyl, C₁₋₆ haloalkyl, C₁₋₆ haloalkoxy, C₁₋₆ haloalkanoyl, amino, C₁₋₆ alkyl amino, di C₁₋₆ alkyl amino, het, phenyl, SF₅ and S(O)ₙR¹¹;
R² is selected from cyano, hydroxy, C(O)OH, het, phenyl, S(O)ₙR¹¹, C(O)NR^{a}R^{b} and C(S)NR^{a}R^{b};
or R² is selected from C₃₋₈ cycloalkyl, C₃₋₈ cycloalkylC₁₋₆ alkyl, C₂₋₆ alkenyl, C₂₋₆ alkynyl, C₁₋₆ alkanoyl, C(O)OC₁₋₆ alkyl, amino, C₁₋₆ alkyl amino, and di C₁₋₆ alkyl amino each of which may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{a} and R^{b} are independently selected from hydrogen, het, phenyl, and S(O)ₙR¹¹;
or either one or both of R^{a} and R^{b} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, and C(O)OC₁₋₆ alkyl, each of which R^{a} or R^{b} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{a} and R^{b} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R² and R^{e} together with the N atom to which R^{e} is attached may form a six to seven-membered saturated, partially saturated, or unsaturated heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R³, R⁴, R⁵ and R⁶ are independently selected from hydrogen, halo, cyano, hydroxy, C(O)OH, nitro, phenyl, and S(O)ₙR¹¹;
or either one or more of R³, R⁴, R⁵ and R⁶ are independently selected from C₁₋₄ alkyl, C(O)NR^{c}R^{d}, C(S)NR^{c}R^{d}, C₁₋₄ alkoxy, C₁₋₄ alkanoyl, C(O)OC₁₋₄ alkyl, amino which R³, R⁴, R⁵ and R⁶ may be optionally and independently further substituted by one or more substituents selected from, where chemically possible, cyano, nitro, halo, hydroxy, C₁₋₄ alkyl and amino;
and where not more than two of R³, R⁴, R⁵ and R⁶ are selected from cyano, hydroxy, C(O)OH, nitro, phenyl, S(O)ₙR¹¹, C(O)NR^{c}R^{d}, C(S)NR^{c}R^{d}, C₁₋₄ alkoxy, C₁₋₄ alkanoyl, C(O)OC₁₋₄ alkyl, and amino;
R⁷ is selected from halo, C₁₋₆ alkyl and C₁₋₆ alkoxy where, when R⁷ is C₁₋₆ alkyl or C₁₋₆ alkoxy, R⁷ may be optionally substituted with one or more halo substituents;
R⁸ is selected from hydrogen, cyano, hydroxy, C(O)OH, nitro, halo, het, phenyl and S(O)ₙR¹¹;
or R⁸ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₁₋₆ alkanoyl, and C(O)OC₁₋₆ alkyl, which R⁸ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R⁸ is amino, which R⁸ may be optionally and independently further substituted by one or more substituents selected from, where chemically possible, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, het, phenyl and S(O)ₙR¹¹;
R⁹ is selected from hydrogen, halo, cyano, hydroxy, C(O)OH, nitro, het, phenyl, S(O)ₙR¹¹ and NR^{e}R^{f};
or R⁹ is selected from C₁₋₆ alkyl, C₂₋₆ alkenyl, C₂₋₆ alkynyl, C₃₋₈ cycloalkyl, C₃₋₈ cycloalkylC₁₋₆ alkyl, C₁₋₆ alkoxy, C₃₋₈ cycloalkylC₁₋₆ alkoxy, C₁₋₆ alkanoyl, C(O)OC₁₋₆ alkyl, which R⁹ may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
R^{e} and R^{f} are independently selected from hydrogen, het, phenyl and S(O)ₙR¹¹;
or either one or both of R^{e} and R^{f} are independently selected from C₁₋₆ alkyl, C₂₋₆ alkenyl, C₃₋₈ cycloalkyl, C₃₋₈ cycloalkylC₁₋₆ alkyl, C₁₋₆ alkanoyl, C(O)OC₁₋₆ alkyl, —C(O)OC₁₋₆ alkylC₃₋₈ cycloalkyl, —C(O)OC₃₋₈ cycloalkyl, each of which R^{e} or R^{f} may be optionally and independently further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R^{f} together with the N atom to which they are attached may form a three to seven-membered saturated, partially saturated, unsaturated or aromatic heterocyclic ring which may optionally contain one or more further N, O or S atoms and which may be optionally further substituted by one or more substituents selected from, where chemically possible, cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, NR^{c}R^{d}, het, phenyl and S(O)ₙR¹¹;
or R^{e} and R² together with the atoms to which they are attached may form a six to seven-membered heterocyclic ring as previously described;
R¹⁰ is selected from halo, C₁₋₆ alkyl and C₁₋₆ alkoxy and where when R¹⁰ is C₁₋₆ alkyl or C₁₋₆ alkoxy it may optionally be substituted with one or more halo substituents;
each of R^{c} and R^{d} are independently selected from hydrogen, C₁₋₆ alkyl, C₂₋₆ alkenyl, C₃₋₈ cycloalkyl, C₃₋₈ cycloalkylC₁₋₆ alkyl, C₁₋₆ haloalkyl, C₃₋₈ cycloalkylC₁₋₆ haloalkyl, C₁₋₆ alkanoyl, C₁₋₆ haloalkanoyl, C(O)OC₁₋₆ alkyl, het, phenyl and S(O)ₙR¹¹;
or R^{c} and R^{d} together with the N atom to which at least one of them is attached may form a three to seven-membered saturated, partially saturated, unsaturated or aromatic heterocyclic ring which may optionally contain one or more further N, O or S atoms;
each n is independently 0, 1 or 2;
each R¹¹ is independently selected from hydrogen, hydroxy, C₁₋₆ alkyl, C₁₋₆ haloalkyl, amino, C₁₋₆ alkyl amino and di C₁₋₆ alkyl amino;
each phenyl may be optionally substituted by one or more further substitutents selected from the group consisting of halo, cyano, nitro, hydroxy, C₁₋₆ alkyl, C₁₋₆ haloalkyl, C₁₋₆ alkoxy, C₁₋₆ haloalkoxy, amino, C₁₋₆ alkyl amino, di C₁₋₆ alkyl amino, —NHS(O)ₙR¹¹, and S(O)ₙR¹¹;
and each het independently represents a four to seven membered heterocyclic ring, which is aromatic or non-aromatic, unsaturated, partially saturated or saturated and which contains one or more heteroatoms selected from nitrogen, N-oxide, oxygen, sulphur and wherein said heterocyclic ring is optionally substituted, where the valence allows, with one or more substituents selected from halo, cyano, nitro, C₁₋₆ alkyl, C₁₋₆ haloalkyl, C₁₋₆ alkoxy, OC(O) C₁₋₆ alkyl, C₁₋₆ alkanoyl, C(O)O C₁₋₆ alkyl and NR^{g}R^{h}, where R^{g} and R^{h} are independently selected from hydrogen, C₁₋₆ alkyl and C₂₋₆ alkenyl, and where each of the above groups may include one or more optional substituents where chemically possible independently selected from cyano, nitro, halo, oxo, hydroxy, C(O)OH, C(O)NR^{c}R^{d}, NR^{c}C(O)R^{d}, C₁₋₆ alkyl, C₂₋₆ alkenyl, C₂₋₆ alkynyl, C₃₋₈ cycloalkyl, C₃₋₈ cycloalkylC₁₋₆ alkyl, C₃₋₈ cycloalkylC₁₋₆ haloalkyl, C₁₋₆ alkoxy, C₁₋₆ alkanoyl, —C(O)OC₁₋₆ alkyl, C₁₋₆ haloalkyl, C₃₋₈ halocycloalkyl, C₁₋₆ haloalkoxy, C₁₋₆ haloalkanoyl, —C(O)OC₁₋₆ haloalkyl, amino, C₁₋₆ alkyl amino, di C₁₋₆ alkyl amino, phenyl and S(O)ₙR¹¹;
or a pharmaceutically acceptable salt or a prodrug thereof; and
b) a pharmaceutical composition comprising a therapeutically effective amount of a second antiparasitic agent.

35. The kit according to claim 34 wherein the second antiparasitic agent is an anthelmintic agent.

36. The kit according to claim 35 wherein the anthelmintic agent is a macrocyclic lactone.

37. The kit according to claim 36 wherein the macrocyclic lactone is a milbemycin or a derivative thereof.

38. The kit according to claim 37 wherein the milbemycin or derivative thereof is milbemycin oxime.

39. The kit according to claim 34 wherein the compound according to formula (i) is cyclopropylmethyl {4-[1-(aminocarbonyl)cyclopropyl]-3-cyano-1-[2,6-dichloro-4-pentafluorothio-phenyl]-1H-pyrazol-5-yl}carbamate.

40. The kit according to claim 39 wherein the second antiparasitic agent is an anthelmintic agent.

41. The kit according to claim 40 wherein the anthelmintic agent is a macrocyclic lactone.

42. The kit according to claim 41 wherein the macrocyclic lactone is a milbemycin or a derivative thereof.

43. The kit according to claim 42 wherein the milbemycin or derivative thereof is milbemycin oxime.
